SEQUENCE LISTING (4 sequences)

SEQ ID NO: 1 - DNA, 2298 bp, Artificial Sequence
  Description of Artificial Sequence; note = synthetic construct
tgtgggtgcggggagtggaattttggaacgaaatgtaacgaagagaagtacagtagtaagagtaacactgtagccgccaccggcaaggggtgcgcgctggggagcggacgctgcatcccctttctgctgcaggaacctctcatcagaccgcctgagggaagcggcgcccggagacccgccccggcccggtccacattctccccaggaagccggactctatggggcgggaccctgggggagcctgagccgagcccggagccagccccgaacccctgaacctccagccaggggcgccccgggagcagccagcccgtgggcgagccgcccgcccgccgagcagccatgagcgagacggtcatctgttccagccgggccactgtgatgctttatgatgatggcaacaagcgatggctccctgctggcacgggtccccaggccttcagccgcgtccagatctaccacaaccccacggccaattcctttcgcgtcgtgggccggaagatgcagcccgaccagcaggtggtcatcaactgtgccatcgtccggggtgtcaagtataaccaggccacccccaacttccatcagtggcgcgacgctcgccaggtctggggcctcaacttcggcagcaaggaggatgcggcccagtttgccgccggcatggccagtgccctagaggcgttggaaggaggtgggccccctccacccccagcacttcccacctggtcggtcccgaacggcccctccccggaggaggtggagcagcagaaaaggcagcagcccggcccgtcggagcacatagagcgccgggtcgccaatgcaggaggcccacctgctccccccgctgggggtccacccccaccaccaggacctccccctcctccaggtccccccccacccccaggtttgcccccttcgggggtcccagctgcagcgcacggagcagggggaggaccaccccctgcaccccctctcccggcagcacagggccctggtggtgggggagctggggccccaggcctggccgcagctattgctggagccaaactcaggaaagtcagcaagcaggaggaggcctcaggggggcccacagcccccaaagctgagagtggtcgaagcggaggtgggggactcatggaagagatgaacgccatgctggcccggagaaggaaagccacgcaagttggggagaaaacccccaaggatgaatctgccaatcaggaggagccagaggccagagtcccggcccagagtgaatctgtgcggagaccctgggagaagaacagcacaaccttgccaaggatgaagtcgtcttcttcggtgaccacttccgagacccaaccctgcacgcccagctccagtgattactcggacctacagagggtgaaacaggagcttctggaagaggtgaagaaggaattgcagaaagtgaaagaggaaatcattgaagccttcgtccaggagctgaggaagcggggttctccctgaccacagggacccagaagacccgcttctcctttccgcacacccggcctgtcaccctgctttccctgcctctacttgacttggaattggctgaagactacacaggaatgcatcgttcccactccccatcccacttggaaaactccaagggggtgtggcttccctgctcacacccacactggctgctgattggctggggaggcccccgcccttttctccctttggtccttcccctctgccatccccttggggccggtccctctgctggggatgcaccaatgaaccccacaggaagggggaaggaaggagggaatttcacattcccttgttctagattcactttaacgcttaatgccttcaaagttttggtttttttaagaaaaaaaaatatatatatatttgggttttgggggaaaagggaaatttttttttctctttggttttgataaaatgggatgtgggagtttttaaatgctatagccctgggcttgccccatttggggcagctatttaaggggaggggatgtctcaccgggctgggggtgagatatccccccaccccagggactccccttccctctggctccttccccttttctatgaggaaataagatgctgtaactttttggaacctcagttttttgattttttatttgggtaggttttggggtccaggccattttttttaccccttggaggaaataagatgagggagaaaggagaaggggaggaaacttctcccctcccaccttcacctttagcttcttgaaaatgggcccctgcagaataaatctgccagtttttataaaaaaaaaa

SEQ ID NO: 2 - protein, 380 aa, Artificial Sequence
  Description of Artificial Sequence; note = synthetic construct
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVANAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP

SEQ ID NO: 3 - protein, 380 aa, Artificial Sequence
  Description of Artificial Sequence; note = synthetic construct
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVSNAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP

SEQ ID NO: 4 - DNA, 2298 bp, Artificial Sequence
  Description of Artificial Sequence; note = synthetic construct
tgtgggtgcggggagtggaattttggaacgaaatgtaacgaagagaagtacagtagtaagagtaacactgtagccgccaccggcaaggggtgcgcgctggggagcggacgctgcatcccctttctgctgcaggaacctctcatcagaccgcctgagggaagcggcgcccggagacccgccccggcccggtccacattctccccaggaagccggactctatggggcgggaccctgggggagcctgagccgagcccggagccagccccgaacccctgaacctccagccaggggcgccccgggagcagccagcccgtgggcgagccgcccgcccgccgagcagccatgagcgagacggtcatctgttccagccgggccactgtgatgctttatgatgatggcaacaagcgatggctccctgctggcacgggtccccaggccttcagccgcgtccagatctaccacaaccccacggccaattcctttcgcgtcgtgggccggaagatgcagcccgaccagcaggtggtcatcaactgtgccatcgtccggggtgtcaagtataaccaggccacccccaacttccatcagtggcgcgacgctcgccaggtctggggcctcaacttcggcagcaaggaggatgcggcccagtttgccgccggcatggccagtgccctagaggcgttggaaggaggtgggccccctccacccccagcacttcccacctggtcggtcccgaacggcccctccccggaggaggtggagcagcagaaaaggcagcagcccggcccgtcggagcacatagagcgccgggtctccaatgcaggaggcccacctgctccccccgctgggggtccacccccaccaccaggacctccccctcctccaggtccccccccacccccaggtttgcccccttcgggggtcccagctgcagcgcacggagcagggggaggaccaccccctgcaccccctctcccggcagcacagggccctggtggtgggggagctggggccccaggcctggccgcagctattgctggagccaaactcaggaaagtcagcaagcaggaggaggcctcaggggggcccacagcccccaaagctgagagtggtcgaagcggaggtgggggactcatggaagagatgaacgccatgctggcccggagaaggaaagccacgcaagttggggagaaaacccccaaggatgaatctgccaatcaggaggagccagaggccagagtcccggcccagagtgaatctgtgcggagaccctgggagaagaacagcacaaccttgccaaggatgaagtcgtcttcttcggtgaccacttccgagacccaaccctgcacgcccagctccagtgattactcggacctacagagggtgaaacaggagcttctggaagaggtgaagaaggaattgcagaaagtgaaagaggaaatcattgaagccttcgtccaggagctgaggaagcggggttctccctgaccacagggacccagaagacccgcttctcctttccgcacacccggcctgtcaccctgctttccctgcctctacttgacttggaattggctgaagactacacaggaatgcatcgttcccactccccatcccacttggaaaactccaagggggtgtggcttccctgctcacacccacactggctgctgattggctggggaggcccccgcccttttctccctttggtccttcccctctgccatccccttggggccggtccctctgctggggatgcaccaatgaaccccacaggaagggggaaggaaggagggaatttcacattcccttgttctagattcactttaacgcttaatgccttcaaagttttggtttttttaagaaaaaaaaatatatatatatttgggttttgggggaaaagggaaatttttttttctctttggttttgataaaatgggatgtgggagtttttaaatgctatagccctgggcttgccccatttggggcagctatttaaggggaggggatgtctcaccgggctgggggtgagatatccccccaccccagggactccccttccctctggctccttccccttttctatgaggaaataagatgctgtaactttttggaacctcagttttttgattttttatttgggtaggttttggggtccaggccattttttttaccccttggaggaaataagatgagggagaaaggagaaggggaggaaacttctcccctcccaccttcacctttagcttcttgaaaatgggcccctgcagaataaatctgccagtttttataaaaaaaaaa

(19) United States
(12) Patent Application Publication
(10) Pub. No.: US 2014/0220018 A1
(43) Pub. Date: Aug. 7, 2014

(54) BIOMARKERS FOR EPITHELIAL CANCER DIAGNOSIS AND TREATMENT

(71) Applicants: Giovanni M. Pitari, Philadelphia, PA (US); David S. Zuzga, Cherry Hill, NJ (US)

(72) Inventors: Giovanni M. Pitari, Philadelphia, PA (US); David S. Zuzga, Cherry Hill, NJ (US)

(21) Appl. No.: 14/126,810

(22) PCT Filed: Jun. 16, 2012
(86) PCT No.: PCT/US12/42840

§ 371 Date: Apr. 3, 2014

Related U.S. Application Data
(60) Provisional application No. 61/497,607, filed on Jun. 16, 2011.

(51) Int. Cl. G01N 33/574

(52) U.S. Cl. CPC G01N 33/57484
USPC 424/135.1; 435/7.1; 435/7.23

(57) ABSTRACT

Disclosed herein are methods for diagnosing the state of an epithelial tumor or tissue in a subject. Also disclosed are methods of diagnosing tumor initiation in an epithelial tissue of a subject. Also disclosed are methods of determining prognosis of a subject with an epithelial tumor. These methods include, in part, measuring the amount of pSer239-VASP, pSer157-VASP, and/or total VASP in a subject or in a tissue or tumor of a subject and comparing those levels to an appropriate reference level. This abstract is intended as a scanning tool for purposes of searching in the particular art and is not intended to be limiting of the present invention.

### CROSS-REFERENCES TO RELATED APPLICATIONS

This application claims benefit of U.S. Provisional Patent Application No. 61/497,607, filed Jun. 16, 2011. The entire contents of U.S. Provisional Patent Application No. 61/497,607 are incorporated herein by reference.

### STATEMENT REGARDING FEDERALLY SPONSORED RESEARCH OR DEVELOPMENT

This invention was made with government support under Grant Number R03CA133950 awarded by the National Institutes of Health. The United States government has certain rights in the invention.

### FIELD OF THE INVENTION

The invention relates to the prognosis, diagnosis, and treatment of epithelial tumors.

### BACKGROUND OF THE INVENTION

Early diagnosis and prognosis are a significant determinant of the success of epithelial cancer treatments. Currently, the methods for diagnosis and prognosis, rely heavily on radiological, histological, and clinical examinations. A need exists for tests that can detect cancer at the earliest stages or determine the risk a patient has of developing cancer. Methods which can be practiced with minimally invasive technologies are particularly useful. Additionally, methods which can offer information regarding the prognosis for a particular subject who has cancer are clinically valuable and a need exists for such tests.

A single type of epithelial cancer, colorectal cancer, is the third most common and third most deadly neoplasm in the western world. Despite advances in identifying the genetic and molecular alterations that characterize epithelial cancers, the causal mechanisms underlying initiation and progression remain unknown.

### SUMMARY

Disclosed herein are methods for diagnosing the state of an epithelial tumor in a subject comprising, measuring the amount of pSer157-VASP, pSer239-VASP, and total VASP in the epithelial tumor; and comparing the amount measured in step a) to an appropriate control amount, to thereby identify the presence or absence of a reduced amount of one or more of the pSer157-VASP, pSer239-VASP, and total VASP in the epithelial tumor; wherein the presence of a reduced amount of pSer239-VASP and the absence of a reduced amount of pSer157-VASP or total VASP in the tumor indicates the state of the tumor is benign, and wherein the presence of a reduced amount of pSer239-VASP coupled with the presence of a reduced amount of one or more of pSer157-VASP and total VASP indicates the state of the tumor is pre-cancerous.

Disclosed herein are methods for diagnosing the state of an epithelial tumor in a subject comprising, measuring the amount of pSer157-VASP, pSer239-VASP, and total VASP in the epithelial tumor; and comparing the amount measured in step a) to an appropriate control amount, to thereby identify the presence or absence of a reduced amount of one or more of the pSer157-VASP, pSer239-VASP, and total VASP in the epithelial tumor; wherein the presence of a reduced amount of pSer239-VASP and the absence of a reduced amount of pSer157-VASP or total VASP in the tumor indicates the state of the tumor is benign, and wherein the presence of a reduced amount of pSer239-VASP coupled with the presence of a reduced amount of one or more of pSer157-VASP and total VASP indicates the state of the tumor is pre-cancerous, wherein the epithelial tumor is removed from the patient prior to step a).

Disclosed herein are methods for diagnosing the state of an epithelial tumor in a subject comprising, measuring the amount of pSer157-VASP, pSer239-VASP, and total VASP in the epithelial tumor; and comparing the amount measured in step a) to an appropriate control amount, to thereby identify the presence or absence of a reduced amount of one or more of the pSer157-VASP, pSer239-VASP, and total VASP in the epithelial tumor; wherein the presence of a reduced amount of pSer239-VASP and the absence of a reduced amount of pSer157-VASP or total VASP in the tumor indicates the state of the tumor is benign, and wherein the presence of a reduced amount of pSer239-VASP coupled with the presence of a reduced amount of one or more of pSer157-VASP and total VASP indicates the state of the tumor is pre-cancerous, further comprising administering a therapeutic composition to the subject.

Disclosed herein are methods for diagnosing the state of an epithelial tumor in a subject comprising, measuring the amount of pSer157-VASP, pSer239-VASP, and total VASP in the epithelial tumor, and determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step a) to that of an appropriate control to identify the presence or absence of a reduced relative amount of pSer239-VASP: total VASP and/or a reduced relative amount of pSer239-VASP: pSer157-VASP and/or a reduced relative amount of pSer157-VASP:total VASP in the epithelial tumor; wherein the presence of a reduced relative amount of pSer239-VASP:total VASP and/or a reduced relative amount of pSer239-VASP: pSer157-VASP, in the absence of a reduced relative amount of pSer157-VASP:total VASP, in the epithelial tumor indicates the state of the tumor is pre-cancerous, and wherein the absence of a reduced relative amount of pSer239-VASP:total VASP and/or a reduced relative amount of pSer239-VASP: pSer157-VASP and/or a reduced relative amount of pSer157-VASP:total VASP in the epithelial tumor indicates the state of the tumor is benign.

Disclosed herein are methods for diagnosing the state of an epithelial tumor in a subject comprising, measuring the amount of pSer157-VASP, pSer239-VASP, and total VASP in the epithelial tumor, and determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step a) to that of an appropriate control to identify the presence or absence of a reduced relative amount of pSer239-VASP: total VASP and/or a reduced relative amount of pSer239-VASP: pSer157-VASP and/or a reduced relative amount of pSer157-VASP:total VASP in the epithelial tumor; wherein the presence of a reduced relative amount of pSer239-VASP:total VASP and/or a reduced relative amount of pSer239-VASP: pSer157-VASP, in the absence of a reduced relative amount of pSer157-VASP:total VASP, in the epithelial tumor indicates the state of the tumor is pre-cancerous, and wherein the absence of a reduced relative amount of pSer239-VASP:total VASP and/or a reduced relative amount of pSer239-VASP: pSer157-VASP and/or a reduced relative amount of pSer157-VASP:total VASP in the epithelial tumor indicates the state of the tumor is benign, wherein the epithelial tumor is removed from the patient prior to step a).

Disclosed herein are methods for diagnosing the state of an epithelial tumor in a subject comprising, measuring the amount of pSer157-VASP, pSer239-VASP, and total VASP in the epithelial tumor, and determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step a) to that of an appropriate control to identify the presence or absence of a reduced relative amount of pSer239-VASP: total VASP and/or a reduced relative amount of pSer239-VASP: pSer157-VASP in the epithelial tumor and/or a reduced relative amount of pSer157-VASP:total VASP; wherein the presence of a reduced relative amount of pSer239-VASP:total VASP and/or a reduced relative amount of pSer239-VASP: pSer157-VASP, in the absence of a reduced relative amount of pSer157-VASP:total VASP, in the epithelial tumor indicates the state of the tumor is pre-cancerous, and wherein the absence of a reduced relative amount of pSer239-VASP:total VASP and/or a reduced relative amount of pSer239-VASP: pSer157-VASP and/or a reduced relative amount of pSer157-VASP:total VASP in the epithelial tumor indicates the state of the tumor is benign, further comprising administering a therapeutic composition to the subject.

Disclosed herein are methods for diagnosing tumor initiation in an epithelial tissue of a subject comprising, measuring the amount of one or more of pSer157-VASP, pSer239-VASP, and total VASP in the epithelial tissue; and identifying the presence or absence of a reduced amount of one or more of pSer157-VASP, pSer239-VASP, and total VASP in the tissue, when compared to an appropriate reference amount; wherein the presence of a reduction of one or more of pSer157-VASP, pSer239-V ASP, and total VASP in the tissue indicates tumor initiation in the epithelial tissue.

Disclosed herein are methods for detecting tumor initiation in an epithelial tissue of a subject comprising, measuring the amount of pSer239-VASP and total VASP, and/or pSer239-VASP and pSer157-VASP, in the epithelial tissue, wherein a reduced ratio of pSer239-VASP:VASP and/or pSer239-VASP:pSer157-VASP, when compared to an appropriate reference level, indicates tumor initiation in the epithelial tissue.

Disclosed herein are methods for determining prognosis of a subject with an epithelial tumor, comprising, measuring the amount of pSer239-VASP, pSer157-VASP, and/or total VASP in the epithelial tumor; and comparing the amount measured in step a) to an appropriate reference amount, to thereby determine the degree of reduction of the pSer239-VASP, pSer157-VASP, and/or total VASP in the epithelial tumor; wherein prognosis is indicated by degree of reduction of the pSer239-VASP, pSer157-VASP, and/or total VASP in the epithelial tumor whereby the greater the degree of reduction, the more negative the prognosis.

Disclosed herein are methods for determining prognosis of a subject with an epithelial tumor, comprising, measuring the amount of pSer157-VASP, pSer239-VASP, and total VASP in the epithelial tumor, and determining the relative amount of pSer239-V ASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step a) to that of an appropriate control to identify the presence or absence of a reduced relative amount of pSer239-VASP:total VASP and/or a reduced relative amount of pSer239-VASP: pSer157-VASP and/or reduced relative amount of pSer157-VASP:total VASP in the epithelial tumor; wherein the presence of a reduced relative amount of pSer239-VASP:total VASP and/or a reduced relative amount of pSer239-VASP: pSer157-VASP, in the presence or absence of reduced relative amount of pSer157-VASP:total VASP, in the epithelial tumor indicates a negative prognosis.

Disclosed herein are methods of determining the likelihood of metastasis of an epithelial tumor in a subject, comprising: measuring the amount of pSer239-VASP, pSer157-VASP, and/or total VASP in the epithelial tumor; and comparing the amount measured in step a) to an appropriate reference amount, to thereby determine the degree of reduction of the pSer239-VASP, pSer157-VASP, and/or total VASP in the epithelial tumor; wherein a greater degree of reduction indicates a higher likelihood of metastasis of the tumor.

Disclosed herein are methods of determining the likelihood of metastasis of an epithelial tumor in a subject, comprising: measuring the amount of pSer157-VASP, pSer239-VASP, and total VASP in the epithelial tumor, and determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step a) to that of an appropriate control to identify the presence or absence of a reduced relative amount of pSer239-VASP:total VASP and/or a reduced relative amount of pSer239-VASP: pSer157-VASP and/or reduced relative amount of pSer157-VASP:total VASP in the epithelial tumor; wherein the presence of a reduced relative amount of pSer239-VASP:total VASP and/or a reduced relative amount of pSer239-VASP: pSer157-VASP and/or reduced relative amount of pSer157-VASP:total VASP in the epithelial tumor indicates a higher likelihood of metastasis of the tumor.

Disclosed herein are methods for treating epithelial neoplasm, in a subject comprising administering to the subject an agent comprising a nucleic acid that encodes S157A-VASP in expressible form, to the subject to thereby deliver the nucleic acid to the neoplastic epithelial cells of the subject.

Disclosed herein are methods for treating epithelial neoplasm, in a subject comprising administering to the subject an agent comprising a nucleic acid that encodes S157A-VASP in expressible form, to the subject to thereby deliver the nucleic acid to the neoplastic epithelial cells of the subject, wherein the epithelial neoplasm is associated with VASP disregulation

Disclosed herein are methods for treating epithelial neoplasm, in a subject comprising administering to the subject an agent comprising a nucleic acid that encodes S157A-VASP in expressible form, to the subject to thereby deliver the nucleic acid to the neoplastic epithelial cells of the subject, wherein the nucleic acid comprises the nucleotide sequence listed in SEQ ID NO: 1.

Disclosed herein are methods for treating epithelial neoplasm, in a subject comprising administering to the subject an agent comprising a nucleic acid that encodes S157A-VASP in expressible form, to the subject to thereby deliver the nucleic acid to the neoplastic epithelial cells of the subject, wherein the S157A-VASP comprises the amino acid sequence listed in SEQ ID NO: 2.

Disclosed herein are methods for diagnosing the state of an epithelial tumor in a subject comprising: transforming pSer157-VASP, pSer239-VASP and total VASP in the epithelial tumor into detectable targets; measuring the amount of each detectable target; and comparing the amount measured in step b) to an appropriate control amount, to thereby identify the presence or absence of a reduced amount of one or more of the pSer157-VASP, pSer239-VASP, and total VASP in the epithelial tumor; wherein the presence of a reduced amount of pSer239-VASP and the absence of a reduced amount of pSer157-VASP or total VASP in the tumor indicates the state of the tumor is benign, and wherein the presence of a reduced amount of pSer239-VASP coupled with the presence of a reduced amount of one or more of pSer157-VASP and total VASP indicates the state of the tumor is pre-cancerous.

Disclosed herein are methods for diagnosing the state of an epithelial tumor in a subject comprising: transforming pSer239-VASP, pSer157-VASP and/or total VASP in the epithelial tumor into detectable targets; measuring the amount of each detectable target; determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step c) to that of an appropriate control to identify the presence or absence of a reduced relative amount of pSer239-VASP:total VASP and/or a reduced relative amount of pSer239-VASP: pSer157-VASP and/or reduced relative amount of pSer157-VASP:total VASP in the epithelial tumor; wherein the presence of a reduced relative amount of pSer239VASP:total VASP and the absence of a reduced ratio of pSer157-VASP: VASP and/or pSer157-VASP:total VASP, when compared to an appropriate reference level, in the tumor indicates the state of the tumor is benign, and wherein the presence of a reduced ratio of pSer239-VASP coupled with the presence of a reduced amount of pSer157-VASP:total VASP, when compared to an appropriate reference level, indicates the state of the tumor is pre-cancerous.

Disclosed herein are methods for diagnosing tumor initiation in an epithelial tissue of a subject comprising, transforming one or more of pSer157-VASP, pSer239-VASP, and total VASP in the epithelial tissue into detectable targets; measuring the level of each detectable target; and identifying the presence or absence of a reduced amount of one or more of pSer157-VASP, pSer239-VASP, and total VASP in the tissue, when compared to an appropriate reference amount; wherein the presence of a reduction of one or more of pSer157-VASP, pSer239-VASP, and total VASP in the tissue indicates tumor initiation in the epithelial tissue.

Disclosed herein are methods for detecting tumor initiation in an epithelial tissue of a subject comprising: transforming pSer239-VASP and total VASP and/or pSer239-VASP and pSer157-VASP in the epithelial tissue into detectable targets; measuring the amount of each detectable target; determining the relative amount of pSer239-VASP:total VASP and/or pSer239-VASP: pSer157-VASP and/or pSer157-VASP:total VASP in the epithelial tissue from the measured amounts; and comparing the relative amount of pSer239-VASP:total VASP and/or pSer239-VASP: pSer157-VASP and/or pSer157-VASP:total VASP determined in step c) to that of an appropriate control to identify the presence or absence of a reduced relative amount of pSer239-VASP:total VASP and/or a reduced relative amount of pSer239-VASP: pSer157-VASP and/or pSer157-VASP:total VASP in the epithelial tumor; wherein the presence of a reduced relative amount of pSer239-VASP: VASP and/or pSer157-VASP:total VASP when compared to an appropriate reference level, indicates tumor initiation in the epithelial tissue.

Disclosed herein are methods for determining prognosis of a subject with an epithelial tumor, comprising, transforming pSer239-VASP, pSer157-VASP and/or total VASP in the epithelial tumor into detectable targets; measuring the amount of each detectable target; and comparing the amount measured in step b) to an appropriate reference amount, to thereby determine the degree of reduction of the pSer239-VASP, pSer157-VASP, and/or total VASP in the epithelial tumor; wherein prognosis is indicated by degree of reduction of the pSer239-VASP, pSer157-VASP, and/or total VASP in the epithelial tumor whereby the greater the degree of reduction, the more negative the prognosis.

Disclosed herein are methods for determining prognosis of a subject with an epithelial tumor, comprising, transforming pSer239-VASP, pSer157-VASP and/or total VASP in the epithelial tumor into detectable targets; measuring the amount of each detectable target; determining the relative amount of pSer239-VASP:total VASP and/or pSer239-VASP: pSer157-VASP and/or pSer157-VASP:total VASP in the epithelial tumor from the amounts measured in b); and comparing the relative amount of pSer239-VASP:total VASP and/or pSer239-VASP: pSer157-VASP and/or pSer157-VASP:total VASP determined in step c) to that of an appropriate control to identify the presence or absence of a reduced relative amount of pSer239-VASP:total VASP and/or a reduced relative amount of pSer239-VASP: pSer157-VASP and/or a reduced relative amount of pSer157-VASP:total VASP in the epithelial tumor; wherein the presence of a reduced relative amount of pSer239-VASP:total VASP and/or a reduced relative amount of pSer239-VASP: pSer157-VASP and/or a reduced relative amount of pSer157-VASP:total VASP in the epithelial tumor indicates a negative prognosis.

Disclosed herein are methods of determining the likelihood of metastasis of an epithelial tumor in a subject, comprising: transforming pSer239-VASP, pSer157-VASP and/or total VASP in the epithelial tumor into detectable targets; measuring the amount of each detectable target; comparing the amount measured in step a) to an appropriate reference amount, to thereby determine the degree of reduction of the pSer239-VASP, pSer157-VASP, and/or total VASP in the epithelial tumor; wherein a greater degree of reduction indicates a higher likelihood of metastasis of the tumor.

Disclosed herein are methods of determining the likelihood of metastasis of an epithelial tumor in a subject, comprising: transforming pSer239-VASP, pSer157-VASP and/or total VASP and/or pSer157-VASP:total VASP in the epithelial tumor into detectable targets; measuring the amount of each detectable target; determining the relative amount of pSer239-VASP:total VASP and/or pSer239-VASP: pSer157-VASP and/or pSer157-VASP:total VASP in the epithelial tumor from the measured amounts; and comparing the relative amount of pSer239-VASP:total VASP and/or pSer239-VASP: pSer157-VASP and/or pSer157-VASP:total VASP determined in step c) to that of an appropriate control to identify the presence or absence of a reduced relative amount of pSer239-VASP:total VASP and/or a reduced relative amount of pSer239-VASP: pSer157-VASP and/or a reduced relative amount of pSer157-VASP:total VASP in the epithelial tumor; wherein the presence of a reduced relative amount of pSer239-VASP:total VASP and/or a reduced relative amount of pSer239-VASP: pSer157-VASP and/or a reduced relative amount of pSer157-VASP:total VASP in the epithelial tumor indicates a higher likelihood of metastasis of the tumor.

Disclosed herein are computer systems for determining the state of an epithelial tissue in a subject, the system comprising: a determination module configured to identify and detect the level of one or more of pSer157-VASP, pSer239-VASP, and/or total VASP in the epithelial tissue; a storage module configured to store output data from the determination module; a comparison module adapted to identify from the output data whether the level of one or more of pSer157-VASP, pSer239-VASP, and/or total VASP in the epithelial tissue is lower than an appropriate reference level; and a display module for displaying whether the level of one or more of pSer157-VASP, pSer239-VASP, and/or total VASP in the epithelial tissue is lower than an appropriate reference level and/or displaying the relative levels of one or more of pSer157-VASP, pSer239-VASP, and/or total VASP.

### BRIEF DESCRIPTION OF THE DRAWINGS

The accompanying figures, which are incorporated in and constitute a part of this specification, illustrate several aspects and together with the description serve to explain the principles of the invention.

FIGS. 1A-1C depict microscopy images demonstrating that VASP Ser phosphorylation is induced by GCC activation in colon cancer cells. FIG. 1A depicts cell leading edges (multi-arrows in upper-left panel) of T84 human colon carcinoma cells (on 2-D surfaces) imaged (magnification, 63×) by D1C and confocal microscopy following immunofluorescent staining with phalloidin (green) and anti-VASP antibody (red). Yellow in merged image indicates F-actin and VASP colocalization at leading tips. FIG. 1B depicts representative confocal microscopy images of invadopodia from T84 cells grown on 3-D Matrigel scaffolds (Matrigel/air interfaces, dotted lines in top panels). Boxes in cartoons on left indicate visual planes in vertical (top panels) and horizontal (middle panels) cross-sections from Z-stack images. Cells were immunofluorescently stained for invadopodial marker cortactin (blue), VASP (red) and F-actin (green). In merged images, white (upper panels) and coincidence of picks (bottom panels) from distinct fluorescent signals along lines depicted in middle panels indicate cortactin, VASP and F-actin colocalization. FIG. 1C depicts vertical or horizontal (bottom-right) T84 cell cross-sections of confocal microscopy images obtained as in FIG. 1B. Here, Matrigel contained DQ-collagen IV, which releases a fluorescent product (green) upon cleavage by extracellular proteases. Application of 60 nM of the broad MMP inhibitor GM6001 (iMMP) was employed to prevent DQ-collagen IV degradation by tumor cells²⁰ and serve as the negative control condition. Red in right panels reflects F-actin staining.

FIGS. 2A-2B depict immunochemical data indicating that VASP Ser phosphorylation is induced by GCC activation in colon cancer cells. FIG. 2A depicts immunoblots of a representative experiment with T84 cells, treated with the GCC ligand ST (1 μM, 5 min) or the cGMP analog 8-br-cGMP (5 mM, 30 min). pS157-VASP and pS239-VASP, VASP phosphorylated at Ser157 and Ser239, respectively; GAPDH, the loading control. FIG. 2B depicts a bar graphs that reflects densitometric quantification of immunobands, normalized to respective loading (GAPDH) and vehicle (PBS) controls (CTR), from experiments with T84 and HCT116 colon cancer cells treated as in FIG. 2A. Some conditions also received the peptide DT2 (5 μM, 30 min) to selectively and completely inhibit PKG-Ia activity (Ki, 12.5 nM;)¹⁷. *, P<0.05, versus respective PBS control.

FIGS. 3A-3C show immunohistochemical data indicating that GCC signaling through VASP is disrupted in colon cancer. FIG. 3A depicts representative immunohistochemistry images (magnification, 20×) of primary tumor (Tumor) and matched normal adjacent tissue (NAT) from 7 patients with colorectal cancer. Tissues were stained with the specific primary antibody (brown) and hematoxylin (blue, nuclei). The primary antibody for VASP phosphorylated at Ser239 was clone 16C2 from Abeam. FIG. 3B is a graph of the distribution (percentages indicated in boxes) of patients with different expression levels (by staining intensity, as described elsewhere herein) of the GCC pathway components. The fraction of patients with upregulation (↑) or downregulation (↓) of the indicated protein in tumors compared to respective NATs is provided in bottom. FIG. 3C depicts a graph of differential protein expression (the delta of staining intensity scores) in colorectal tumors compared to matched NATs. *, P<0.05; ***, P<0.005 of tumors versus NATs. pS157-VASP and pS239-VASP, VASP phosphorylated at Ser157 and Ser239, respectively.

FIGS. 4A-4C depict graphs indicating that VASP and VASP Ser phosphorylation are biomarkers for colon cancer tumorigenesis. Human colon specimens (NT=normal tissues, HP=hyperplastic polyp, TA=tubular adenoma) were subjected to immunohistochemistry employing specific antibodies for human VASP (dilution, 1:100), pSer157-VASP (pS157-VASP; dilution, 1:100) or pSer239-VASP (pS239-VASP; dilution, 1:100). FIG. 4A is a graph of the means±SEM of staining scores. Numbers in columns represent the n of human cases analyzed. FIG. 4B depicts a different graph representation of the data shown in FIG. 4A. FIG. 4C depicts a graph of pVASP/VASP ratios (means±SEM) of staining scores assigned to colorectal tubular adenomas (TA) and normal adjacent tissues (NAT) from 5 patients. *, p<0.05; **, p<0.01 and***, p<0.001 by 2-tailed t test.

FIGS. 5A-5C depict graphs indicating that pSer239-VASP is a prognostic indicator of colorectal cancer metastasis. FIG. 5A-5B depict data from human colon adenocarcinomas (Cancer) and normal adjacent tissues (NAT) which were subjected to IHC employing a rabbit anti-human pSer239-VASP antibody (pS239-VASP; dilution, 1:600). Values are means±SEM, Numbers in parenthesis below x axis labels reflect n of cases analyzed. FIG. 5A shows staining scores in normal vs adenocarcinoma samples. FIG. 5B shows staining scores for different clinical sub-groups (NO=primary colorectal cancer adenocarcinomas with tumor negative lymph nodes; N1/N2=primary colorectal cancer adenocarcinomas with tumor positive lymph nodes). FIG. 5C depicts the percentage of epithelial cell compartments (%) with pSer239-VASP loss in different clinical sub-groups. **, p<0.01 and ***, p<0.001 by one-way ANOVA with post-test for linear trend.

FIGS. 6A-6C depict microscopy data indicating that filopodia are impaired by cGMP signaling. FIG. 6A depicts filopodia (arrows in upper-left slide) imaged in cancer cell colonies by DIC. The top row received a PBS control treatment. The bottom row received activators of cGMP, either the GCC ligand ST (1 μM), or 8-bromo-cGMP (5 mM). FIGS. 6B and 6C depict graphs of the data shown in FIG. 6A, quantifying, respectively, frequency and length of filopodia. *, P<0.05, versus respective PBS control.

FIGS. 7A-7C depict confocal microscopy data indicating that invadopodia are impaired by cGMP signaling. FIG. 7A depicts invadopodia imaged by confocal microscopy in single T84 cells, immunofluorescently stained with 4′,6-diamidino-2-phenylindole (DAPI, for nuclei in blue) and phalloidin (for F-actin in green). Boxes in cartoons on top indicate visual planes in vertical (left panels) and horizontal (right panels) cross-sections from Z-stack images. The top row received a PBS control treatment. The bottom row received the GCC ligand ST (1 μM), FIGS. 7B-7C depict graphs of the data shown in FIG. 7A, quantifying, respectively, frequency and length of invadopodia. *, P<0.05, versus respective PBS control.

FIGS. 8A-8C depict microscopy data indicating that VASP Ser239 phosphorylation by GCC disrupts colon cancer cell filopodia. T84 cells were treated (with the PBS control or 1 μM ST to induce GCC signaling), imaged and analyzed for quantification of filopodia. Cancer cells stably expressed the indicated MSCV-driven genes, including the controls GFP and VASP, and VASP-S157A (S157A), VASP-S239A (S239A) and VASP-AA (AA). FIG. 8A depicts DIC images, arrows indicate representative filopodia. Bars, 10 μm. FIGS. 8B and 8C depict graphs of the data shown in FIG. 6A, quantifying, respectively, frequency and length of filopodia. *, P<0.05 versus respective control.

FIGS. 9A-9C depict confocal microscopy data indicating that VASP Ser239 phosphorylation by GCC disrupts colon cancer cell invadopodia. T84 cells were treated (with the PBS control or 1 μM ST to induce GCC signaling), imaged and analyzed for quantification of invadopodia. Cancer cells stably expressed the indicated MSCV-driven genes, including the control VASP, and VASP-S157A (S157A), VASP-S239A (S239A) and VASP-AA (AA). FIG. 9A depicts confocal microscopy images, boxes in cartoons on left of FIG. 9A indicate visual planes in vertical (upper panels) and horizontal (bottom panels) cross-sections from Z-stacks. FIGS. 9B-9C depict graphs of the data shown in FIG. 9A, quantifying, respectively, frequency and length of invadopodia. *, P<0.05 versus respective control.

FIGS. 10A-10B depict microscopy images indicating that VASP Ser239 phosphorylation induces membrane protrusion retraction in colon cancer cells. Fluorescently-tagged VASP constructs, GFP-VASP (VASP) and GFP-VASP-S239A (S239A), were stably expressed in T84 cells. GCC signaling was induced with ST (1 μM). while PBS was the vehicle control. FIG. 10A depicts representative time course of a live cell experiment analyzing filopodial kinetics by confocal microscopy. Photographs reflect merged DIC and confocal microscopy images. FIG. 10B depicts representative 3-D projections from live imaging (confocal microscopy) analyzing invadopodia in single tumor cells plated on Matrigel scaffolds. Cartoons on top indicate visual vertical (left panels) and basolateral en face (right panels) perspectives of 3-D reconstructions from Z-stack images.

FIGS. 11A-11C depict graphs indicating that VASP Ser239 phosphorylation induces membrane protrusion retraction in colon cancer cells. FIG. 11A is a graph of the length of filopodia following treatments for 15 min, expressed as percentages of respective PBS controls. GCC signaling was induced with ST (1 μM), while PBS was the vehicle control. FIG. 11B is a graph quantifying the GFP-tagged VASP signals at filopodia tips from experiments shown in FIG. 10A, Results are percentages of respective time 0 (before ST administration) controls. FIG. 11C is a graph quantifying GFP-tagged VASP signals in cell bodies (CB) and filopodia (F) before (CTR, time 0 control) and after ST (15 min) treatments, as shown in FIG. 10A. AU, arbitrary units. *, P<0.05 versus respective control.

FIGS. 12A-12B depict confocal microscopy data indicating that VASP Ser239 phosphorylation inhibits the cleavage of DQ-collagen IV by colon cancer cells. T84 cells stably-expressing the indicated MSCV-driven genes (VASP, the control; VASP-S157A, S157A; VASP-S239A, S239A; VASP-AA, AA) were treated for 24 h with PBS, 1 μM ST or 60 nM of the broad MMP inhibitor GM6001 (iMMP), imaged and analyzed by confocal microscopy for quantification of DQ-collagen IV degradation. FIG. 12A depicts photographs which are merged DIC and confocal microscopy images. FIG. 12B depicts a graph quantifying the results, expressed as arbitrary units (AU) of the fluorescent signal from the cleaved collagen product. WT, wild-type T84 cells. *, P<0.05, versus respective PBS control.

FIGS. 13A-13B depict in vivo experiments indicating that pSer239-VASP inhibits peritoneal metastasis by colon cancer cells in mice. Following ST treatments (1 μM; 24 h), T84 cells (1×107) were injected intraperitoneally into nude CD-1 mice (male, 5 wk-old). FIG. 13A depicts representative images of peritoneal metastasis after 12 wk. FIG. 13B depicts graphs quantifying peritoneal carcinomatosis using a modified clinical index, reflecting the sum of scores from 10 regions of the peritoneal cavity. Scores were: 0, no visible tumors; 1, tumors<0.5 mm; 2, tumors>0.5-<1.5 mm, and 3, tumors>1.5 mm. ST inhibits colon cancer cell metastasis (left panel; PBS control group, n=12; ST group, n=16) by inducing pSer239-VASP, as assessed with T84 cells expressing the specific alanine-substituted VASP mutant (S239A in right panel; PBS group, n=10; ST group, n=9). Values, means±SEM. *, p<0.05 by t test.

FIGS. 14A-14B depict a graph and a table indicating that VASP phosphomutant S157A is a novel therapeutic agent for colon cancer. FIG. 14A is a graph of fitted logistic growth curves generated by counting T84 cells expressing VASP phosphomutants over a 12-day time course. FIG. 14B is a table demonstrating statistical comparisons of growth parameters with significance in red text. Columns are growth rate, the slope of the log-linear phase; inflection point, cell number at ½ maximal capacity (cell confluency).

FIGS. 15A-15B depict graphs indicating that VASP phosphomutant S157A is a novel therapeutic agent for colon cancer. DNA synthesis by 3H-thymidine incorporation assay in T84 cells expressing the VASP phosphomutants. ST treatments were 1 μM for 3 h (FIG. 15B). Values (means±SEM) reflect % of respective empty vector (MSCV)(FIG. 15A) or vehicle (PBS) control (the O-normalized value in graphs). ***, p<0.001 by t test.

FIG. 16 is an illustration of the proposed role of VASP in colorectal carcinogenesis. During tumor progression, VASP phosphorylation at Ser239 (p-Ser239-VASP) is reduced, reflecting a dysregulated GCC pathway with interruption of cGMP-mediated signaling, which promotes the formation of migratory membrane protrusions and cancer cell invasion. Reconstitution of GCC and cGMP signaling, in turn, provides a unique therapeutic opportunity to restrain the invasive tumor cell shape and prevent metastasis.

FIG. 17 is a diagram of an embodiment of a system for performing a method for determining the presence of epithelial cancer in a subject by measuring the level of pSer157-VASP, pSer239-VASP, and/or total VASP in a sample obtained from a subject.

FIG. 18 is a diagram of an embodiment of a comparison module as described herein.

FIG. 19 is a diagram of an embodiment of an operating system and applications for a computing system as described herein.

FIGS. 20A-20B are images of immunoblots demonstrating that ligand-dependent GCC signaling induces rapid VASP Ser phosphorylation in colon cancer cells. FIG. 20A depicts VASP-specific immunoblots of representative experiments (repeated 3 times) analyzing total cell lysates from various human colon carcinomas. GAPDH is the loading control. FIG. 20B depicts immunoblots of representative time-course experiments with T84 human colon carcinoma cells treated with the GCC agonist (1 μM). pS157-VASP and pS239-VASP, VASP phosphorylated at Ser157 and Ser239, respectively.

FIGS. 21A-21B are images of immunoblots demonstrating that GCC signaling through PKG induces VASP Ser phosphorylation in colon cancer cells. FIG. 21A depicts immunoblots of representative experiments with HCT116 human colon carcinoma cells. FIG. 21B depicts immunoblots of representative experiments with and T84 human colon carcinoma cells. Treatments were: the cGMP analog 8-br-cGMP (5 mM, 30 min); the GCC ligand ST (1 μM, 5 min), and the selective PKG inhibitor DT2 (5 μM, 30 min;)¹⁷. pS157-VASP and pS239-VASP, VASP phosphorylated at Ser157 and Ser239, respectively; GAPDH, the loading control.

FIG. 22 is a series of photographs of immunohistochemistry demonstrating that GCC signaling through VASP Ser phosphorylation is dysregulated in colon cancer. IHC images (magnification, 20×) of serial sections from primary tumor (Tumor) and matched normal adjacent tissue (NAT) of a patient with colorectal cancer. Tissues were stained with the specific primary antibody (brown) and hematoxylin (blue, nuclei). A rabbit primary antibody (1:600 dilution) from Sigma-Aldrich was employed for VASP phosphorylated at Ser239. pS157-VASP and pS239-VASP, VASP phosphorylated at Ser157 and Ser239, respectively.

FIGS. 23A-23B are immunoblots demonstrating that colon cancer cells with VASP phosphomutants are resistant to GCC-mediated phosphorylation. FIG. 23A depicts representative immunoblots of total VASP (VASP) and the loading control (GAPDH) from T84 cells stably expressing the indicated MSCV-driven genes, including VASP-S157A (S157A), VASP-S239A (S239A) and VASP-AA (AA). Wild-type (WT) T84 cells, and cells expressing GFP or VASP represent the control conditions. FIG. 23B depicts representative immunoblots and associated graphs (reflecting mean±SEM of 3 independent experiments) of VASP phosphorylated at Ser157 (pS157-VASP; left) and VASP phosphorylated at Ser239 (pS239-VASP; right) in T84 cells (genetically manipulated as in a) treated for 5 min with ST (1 μm), Values in graphs are relative levels normalized to respective loading (villin) and vehicle (PBS) controls. *, P<0.05, versus respective PBS control.

FIG. 24 is a graph demonstrating that GCC signaling induces rapid VASP removal from colon cancer cell filopodia. Exponential decay curve fitted to the data presented in FIG. 11B, examining the effects of the GCC agonist ST (1 μM) on VASP localization at filopodia. Dotted lines are 95% confidence limits. The curve was generated employing the software Prism 5.0 (GraphPad).

### DETAILED DESCRIPTION OF THE INVENTION

The present invention can be understood more readily by reference to the following detailed description of the invention and the Examples included therein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Methods of Diagnosis and Prognosis

Aspects of the present invention stem from the finding that the phosphorylation state of the VASP protein in an epithelial tissue can be used to monitor epithelial tumor initiation and progression in that tissue. In addition to this, the amount of total VASP protein present in an epithelial tissue can also be used to monitor epithelial tumor initiation and progression in that tissue. More specifically, a comparatively lower amount of VASP phosphorylated at pSer157, or VASP phosphorylated at pSer239, or of total VASP occurs at pre-determined states of epithelial tumor initiation and progression. Analysis of the amount of the different VASP phosphoisomers and of total VASP, independently, or in combination, as determined in comparison to the amount of the same respective proteins/phosphoisomers in an appropriate control tissue, can be used to determine the neoplastic state of one or more epithelial cells (e.g., a suspected tumor or pretumor mass). As such, a threshold amount of reduction in VASP phosphorylated at pSer157, or VASP phosphorylated at pSer239, or of total VASP, and combination thereof, can be used to detect tumor initiation in an epithelial tissue. It also follows that determination of a threshold amount of reduction in VASP phosphorylated at pSer157, or VASP phosphorylated at pSer239, or of total VASP. and combination thereof, in the epithelial cells of a subject (e.g., obtained from a biological sample) can be used in diagnosis of the subject, to indicate the presence of a tumor in the subject. An absence of a reproducible, statistically significant reduction in pSer157-VASP, pSer239-VASP, or of total VASP, indicates the absence of tumor initiation. The presence of a reproducible, statistically significant reduction in one or more of pSer157-VASP, pSer239-VASP, or of total VASP indicates tumor initiation.

Using immunohistochemical analysis of the different phosphorylated forms of VASP in epithelial cells, changes in the levels of pSer157-VASP, pSer239-VASP, and total VASP that accompany tumor initiation and metastasis have been identified. Results of experiments detailed in the Examples section below indicate that as epithelial tissue undergoes the progression from normal tissue, to a benign tumor, to a malignant tumor, to a tumor with invasive potential and metastasis, the levels of pSer157-VASP, pSer239-VASP, and total VASP decrease. Accordingly, the level of pSer157-VASP, pSer239-VASP, and total VASP can be used for diagnosis and/or prognosis of epithelial neoplasm in a subject. In some embodiments, pSer157-VASP, pSer239-VASP, and total VASP are detected using protein expression analysis.

In some embodiments, a certain percentage reduction in the level of pSer157-VASP, pSer239-VASP, and/or total VASP is indicative of the presence of a benign tumor, a malignant tumor, or a invasive tumor that is likely to metastasize or is metastasizing.

In some embodiments, an epithelial tumor or epithelial tissue is removed from the patient prior to measuring the amount of pSer157-VASP, pSer239-VASP, and total VASP.

In some embodiments, the methods disclosed herein further comprise administering a therapeutic composition to the subject after the amount of pSer157-VASP, pSer239-VASP, and total VASP is measured or after the state of an epithelial tumor in the subject is diagnosed.

A scale of prognostic significance of the reduced amounts of VASP phosphorylated at pSer157, or VASP phosphorylated at pSer239, or of total VASP, is provided in Table 1 below. This scale has been produced by the methods described herein. Similar scales can be generated using alternative forms of measurement of the different VASP isoforms. In one embodiment, a detectable reduction in pSer157-VASP, pSer239-VASP, or of total VASP that is less than about a 35% reduction, as determined by the provided methods, or comparable methods, is indicative of tumor initiation, with the tumor being benign.

TABLE 1
Scale of Prognostic Significance of each of the VASP Species in
Epithelial Neoplasia, as determined from Colorectal Cancer Analysis. Ranges of
percentages of reduction in the staining intensity of any one of the VASP forms (e.g., total
VASP; pSer157-VASP; pSer239-VASP) in the sample compared to the reference control (the
correspondent normal adjacent tissue). NS, not significant (=not statistically different from
the control).

| | | | Malignant Tumor; | Invasive |
|---|---|---|---|---|
| | Normal | Benign | Cancer without In | Cancer; |
| | Mucosa | Tumor | vasion/Metastasis | Metastasis |
| Staining | 0; NS | >0 to <35 | >35 to <50 | >50 |
| Intensity | | | | |
| Reduction, % | | | | |
| Indication | No Tumor | Tumor | Tumor Progression | Tumor |
| | | Initiation | | Metastasis |

The information in Table 1 can be used in any of the methods disclosed herein. For example, the level of pSer 157-VASP, pSer239-VASP either individually, in combination, or the ratio of one or more of the two phosphorylated VASP forms to total VASP can be used in the methods disclosed herein. In some embodiments, a reduction in the overall level of pSer 157-VASP, pSer239-VASP, or total VASP can indicate tumor stage. In some embodiments, the ratio of pSer 157-VASP or pSer239-VASP to total VASP can be used.

In some embodiments, a reduction in the level of Ser 157-VASP, pSer239-VASP, and/or total VASP of no more than 35% as compared to the reference indicates the presence of a benign tumor. In some embodiments, a reduction in the level of Ser157-VASP, pSer239-VASP, and/or total VASP of no more than 35% as compared to the reference indicates the subject does not have epithelial cancer. In some embodiments, a reduction in the level of Ser157-VASP, pSer239-VASP, and/or total VASP of no more than 35% as compared to the reference indicates a higher likelihood of the subject developing epithelial cancer. In some embodiments, a reduction in the level of Ser157-VASP, pSer239-VASP, and/or total VASP of no more than 35% as compared to the reference indicates that the subject can benefit from treatment to prevent the development of cancer.

In some embodiments, a reduction in the ratio of pSer 157-VASP or pSer239-VASP, to total VASP of no more than 35% as compared to the reference indicates the presence of a benign tumor. In some embodiments, a reduction in the ratio of pSer 157-VASP or pSer239-VASP, to total VASP of no more than 35% as compared to the reference indicates the subject does not have epithelial cancer. In some embodiments, a reduction in the ratio of pSer 157-VASP or pSer239-VASP, to total VASP of no more than 35% as compared to the reference indicates a higher likelihood of the subject developing epithelial cancer. In some embodiments, a reduction in the ratio of pSer 157-VASP or pSer239-VASP, to total VASP of no more than 35% as compared to the reference indicates that the subject can benefit from treatment to prevent the development of cancer.

In some embodiments, a reduction in the level of Ser 157-VASP, pSer239-VASP, and/or total VASP of no more than 35% as compared to the reference indicates the presence of a benign tumor. In some embodiments, a reduction in the level of Ser157-VASP, pSer239-VASP, and/or total VASP of about 30% to 55% as compared to the reference indicates the subject does not have epithelial cancer. In some embodiments, a reduction in the level of Ser157-VASP, pSer239-VASP, and/or total VASP of no more than 30%-35% as compared to the reference indicates a higher likelihood of the subject developing epithelial cancer. In some embodiments, a reduction in the level of Ser157-VASP, pSer239-VASP, and/or total VASP of no more than 30%-35% as compared to the reference indicates that the subject can benefit from treatment to prevent the development of cancer.

In some embodiments, a reduction in the ratio of pSer 157-VASP or pSer239-VASP, to total VASP of no more than 35% as compared to the reference indicates the presence of a benign tumor. In some embodiments, a reduction in the ratio of pSer 157-VASP or pSer239-VASP, to total VASP of about 30% to 55% as compared to the reference indicates the subject does not have epithelial cancer. In some embodiments, a reduction in the ratio of pSer 157-VASP or pSer239-VASP, to total VASP of no more than 30%-35% as compared to the reference indicates a higher likelihood of the subject developing epithelial cancer. In some embodiments, a reduction in the ratio of pSer 157-VASP or pSer239-VASP, to total VASP of no more than 30%-35% as compared to the reference indicates that the subject can benefit from treatment to prevent the development of cancer.

In some embodiments, a reduction in the level of pSer157-VASP, pSer239-VASP, and/or total VASP equal to or more than 35% and less than 50% as compared to the reference indicates the presence of a malignant tumor. In some embodiments, a reduction in the level of pSer157-VASP, pSer239-VASP, and/or total VASP equal to or more than 35% and less than 50% as compared to the reference indicates the presence of a malignant tumor that is not invasive. In some embodiments, a reduction in the level of pSer157-VASP, pSer239-VASP, and/or total VASP equal to or more than 35% and less than 50% as compared to the reference indicates the presence of a malignant tumor that is not metastasizing and/or capable of metastasizing. In some embodiments, a reduction in the level of pSer157-VASP, pSer239-VASP, and/or total VASP equal to or more than 35% and less than 50% as compared to the reference indicates the subject has an epithelial cancer. In some embodiments, a reduction in the level of pSer157-VASP, pSer239-VASP, and/or total VASP equal to or more than 35% and less than 50% as compared to the reference indicates an increased risk of the subject developing an invasive tumor. In some embodiments, a reduction in the level of pSer157-VASP, pSer239-VASP, and/or total VASP equal to or more than 35% and less than 50% as compared to the reference indicates an increased risk of the subject developing a metastatic tumor. In some embodiments, a reduction in the level of pSer157-VASP, pSer239-VASP, and/or total VASP equal to or more than 35% and less than 50% as compared to the reference indicates the subject is in need of treatment for epithelial cancer. In some embodiments, a reduction in the level of pSer157-VASP, pSer239-VASP, and/or total VASP equal to or more than 35% and less than 50% as compared to the reference indicates the subject is in need of treatment to prevent development of a metastasizing or invasive epithelial cancer.

In some embodiments, a reduction in the ratio of pSer 157-VASP or pSer239-VASP, to total VASP equal to or more than 35% and less than 50% as compared to the reference indicates the presence of a malignant tumor. In some embodiments, a reduction in the ratio of pSer 157-VASP or pSer239-VASP, to total VASP equal to or more than 35% and less than 50% as compared to the reference indicates the presence of a malignant tumor that is not invasive. In some embodiments, a reduction in the ratio of pSer 157-VASP or pSer239-VASP, to total VASP equal to or more than 35% and less than 50% as compared to the reference indicates the presence of a malignant tumor that is not metastasizing and/or capable of metastasizing. In some embodiments, a reduction in the ratio of pSer 157-VASP or pSer239-VASP, to total VASP equal to or more than 35% and less than 50% as compared to the reference indicates the subject has an epithelial cancer. In some embodiments, a reduction in the ratio of pSer 157-VASP or pSer239-VASP, to total VASP equal to or more than 35% and less than 50% as compared to the reference indicates an increased risk of the subject developing an invasive tumor. In some embodiments, a reduction in the ratio of pSer 157-VASP or pSer239-VASP, to total VASP equal to or more than 35% and less than 50% as compared to the reference indicates an increased risk of the subject developing a metastatic tumor. In some embodiments, a reduction in the ratio of pSer 157-VASP or pSer239-VASP, to total VASP equal to or more than 35% and less than 50% as compared to the reference indicates the subject is in need of treatment for epithelial cancer. In some embodiments, a reduction in the ratio of pSer 157-VASP or pSer239-VASP, to total VASP equal to or more than 35% and less than 50% as compared to the reference indicates the subject is in need of treatment to prevent development of a metastasizing or invasive epithelial cancer.

In some embodiments, a reduction in the level of pSer157-VASP, pSer239-VASP, and/or total VASP equal to or more than 50% as compared to the reference indicates the presence of a malignant invasive tumor. In some embodiments, a reduction in the level of pSer157-VASP, pSer239-VASP, and/or total VASP equal to or more than 50% as compared to the reference indicates the presence of a malignant tumor likely to metastasize. In some embodiments, a reduction in the level of pSer157-VASP, pSer239-VASP, and/or total VASP equal to or more than 50% as compared to the reference indicates the subject has epithelial cancer. In some embodiments, a reduction in the level of pSer157-VASP, pSer239-VASP, and/or total VASP equal to or more than 50% as compared to the reference indicates the subject is likely to develop secondary tumors or has secondary tumors. In some embodiments, a reduction in the level of pSer157-VASP, pSer239-VASP. and/or total VASP equal to or more than 50% as compared to the reference indicates the presence of a malignant invasive tumor. In some embodiments, a reduction in the level of pSer157-VASP, pSer239-VASP, and/or total VASP equal to or more than 50% as compared to the reference indicates the subject is in need of treatment for epithelial cancer. In some embodiments, a reduction in the level of pSer157-VASP, pSer239-VASP, and/or total VASP equal to or more than 50% as compared to the reference indicates the subject is in need of treatment for metastasis of an epithelial cancer.

In some embodiments, a reduction in the ratio of pSer 157-VASP or pSer239-VASP, to total VASP equal to or more than 50% as compared to the reference indicates the presence of a malignant invasive tumor. In some embodiments, a reduction in the ratio of pSer 157-VASP or pSer239-VASP, to total VASP equal to or more than 50% as compared to the reference indicates the presence of a malignant tumor likely to metastasize. In some embodiments, a reduction in the ratio of pSer 157-VASP or pSer239-VASP, to total VASP equal to or more than 50% as compared to the reference indicates the subject has epithelial cancer. In some embodiments, a reduction in the ratio of pSer 157-VASP or pSer239-VASP, to total VASP equal to or more than 50% as compared to the reference indicates the subject is likely to develop secondary tumors or has secondary tumors. In some embodiments, a reduction in the ratio of pSer 157-VASP or pSer239-VASP, to total VASP equal to or more than 50% as compared to the reference indicates the presence of a malignant invasive tumor. In some embodiments, a reduction in the ratio of pSer 157-VASP or pSer239-VASP, to total VASP equal to or more than 50% as compared to the reference indicates the subject is in need of treatment for epithelial cancer. In some embodiments, a reduction in the ratio of pSer 157-VASP or pSer239-VASP, to total VASP equal to or more than 50% as compared to the reference indicates the subject is in need of treatment for metastasis of an epithelial cancer.

In addition to the absolute levels of the different forms of VASP (VASP phosphorylated at Ser157, VASP phosphorylated at Ser239, or of total VASP), the experiments presented herein further indicate that the relative amount of the different forms of VASP to one another in an epithelial cell or tissue, can be used to monitor epithelial tumor initiation and progression. More specifically, a lower relative amount of pSer239-VASP to total VASP, and/or a lower relative amount of pSer239-VASP to pSer157-VASP, in the absence of lower relative amount of pSer157-VASP:total VASP, as determined by comparison of the respective relative amounts in an appropriate control cell or tissue, occurs at pre-determined states of epithelial tumor initiation and progression. As such, analysis of the relative amounts of pSer157, pSer239, and total VASP, can be used to determine the neoplastic state of one or more epithelial cells (e.g., a suspected tumor or pretumor mass). A threshold amount of reduction in one or more relative amounts (pSer239-VASP: total VASP, and/or pSer239-VASP: pSer157-VASP) in epithelial cells or tissue comparison to that of an appropriate control, can be used to detect tumor initiation in the epithelial tissue. It also follows that analysis of the relative amounts of pSer157, pSer239, and total VASP, in the epithelial cells or tissue of a subject (e.g., obtained from a biological sample) can be used to diagnose the presence of a tumor in a subject.

As the term is used herein, “a reduced amount or level”, refers to an identified, reproducible, quantitative or qualitative reduction in the physical presence of a target molecule or molecules (e.g., a specific phosphorylated isoform of VASP or total VASP). A reduced amount is determined by measuring (e.g., quantitative) the target molecule(s) in a target cell or tissue, to produce a determined amount, followed by comparison to a determined control amount obtained by measuring of the target molecule(s) in an appropriate control cell or tissue, under normal or non-disease conditions. Measurement in the target cell or tissue and in the control cell or tissue is performed by as close to identical methods as possible under the given experimental conditions.

Measurement of the physical presence of a molecule typically involves transformation of the target molecule(s), or another indicator, present in the cell, to an indicator molecule, as described herein. The presence of the indicator molecule is then measured or otherwise quantitatively detected (e.g., by a non-human machine).

As the term is used herein, a “reduced relative amount” refers to an identified, reproducible, quantitative or qualitative, reduction in the relative amount of the physical presence of a target entity, as compared to another target entity (e.g., pSer239-VASP:pSer157-VASP, or pSer239-VASP:total VASP, or pSer157-VASP:total VASP). The relative amount of the respective entities is determined by quantitative measurement of the two entities (e.g., a specifically phosphorylated molecules in a target cell or tissue) by appropriately comparable methods of detection, with calculation of the relative amounts by standard mathematical means (e.g., determination of the ratio of the two determined measurements) to produce a quantitative measurement reflective of the relative amount of the two entities. The identical methods are used to measure the relative amount of the same respective entities in an appropriate control cell or tissue, to produce a quantitative measurement reflective of the relative amount of the two entities under normal, non-disease conditions. A reduction in the relative amount is identified by comparison of the relative amount calculated from the target tissue, to the relative amount calculated from the control tissue, wherein a reproducible, statistically significant, lower relative amount is quantitatively or qualitatively determined.

As the term is used herein, total VASP refers to all mature, processed VASP protein in a cell, regardless of the phosphorylation state. It is inclusive of pSer157 and of pSer239 VASP, as well as other potential variations in the phosphorylation state of the protein.

Tumor progression from benign neoplasm to malignancy (in the absence of invasion/metastasis) and then to invasiveness (with metastasis) further has been observed to consistently correspond to a continuum of reduction of the different VASP phosphoisomers as well as total VASP. As such, with respect to tumor progression in a subject, the amount of the different VASP phosphoisomers and of total VASP, independently, or in combination, as discussed above, and also the relative amount of the different forms of VASP to one another, in an epithelial tumor, are also indicative of the progression of the tumor into malignancy. As such, the degree of reduction in the absolute amount, or in the relative amount, of the VASP phosphoisomers, discussed above, indicates how far the tumor has progressed.

The scale of prognostic significance of the VASP phosphorylated at pSer157, or VASP phosphorylated at pSer239, or of total VASP, provided in Table 1, as determined by the methods described herein, further indicates relevant values for determining and monitoring tumor progression. A reduction in pSer157-VASP, pSer239-VASP, or of total VASP that is greater than or equal to about 35%, and less than about 50%, as determined by the provided methods, or comparable methods, is indicative of the tumor being malignant (without the progression to an invasive state). A reduction in pSer157-VASP, pSer239-VASP, or of total VASP that is greater than or equal to about 50%, 55%, or 60% as determined by the provided methods, or comparable methods, is indicative of tumor progression from malignant (without the progression to an invasive state) to invasive.

A greater degree of reduction in the amount of the different VASP phosphoisomers and of total VASP, independently, or in combination, along a continuum of reduction, is used to indicate progression of the tumor toward malignancy (e.g., from a detectable amount of reduction to less than about 35% reduction, wherein a reduction of about 35%, 40%, 45%, or 50% or more indicates the tumor is no longer considered benign). An even greater reduction along the same continuum is used to indicate the progression of the tumor toward invasiveness, correlating with metastasis (e.g, from about 35% to less than about a 50% reduction, wherein a reduction of about 50%, 55% or 60% indicates the tumor is considered to be invasive). An even greater reduction along the same continuum is used to indicate how strongly invasive or metastatic the tumor is considered (starting with a reduction of about 50%, 55% or 60% to indicate invasiveness, with further reductions indicating a propensity for even more aggressive invasion.

Similarly, a greater reduction in one or more relative amounts of the VASP isoforms (e.g., pSer239-VASP: total VASP, and/or pSer239-VASP: pSer157-VASP, with/or without a greater reduction in pSer157-VASP:total VASP) along a continuum of reduction, is used to indicate progression of the tumor to malignancy. An even greater reduction, along the same continuum, is used to indicate the progression of the tumor to invasiveness, correlating with metastasis, and an even greater reduction, along the same continuum, is used to indicate how strongly invasive or metastatic the tumor is considered.

How far the tumor has progressed with respect to malignancy, and invasiveness, in turn indicates prognosis of the subject with the tumor. As such, another aspect of the invention relates to a method for determining the prognosis of a subject with an epithelial tumor, by determining the degree of reduction of the amount of the different VASP phosphoisomers and of total VASP, independently, or in combination, and/or determining the degree of reduction in one or more relative amounts of the VASP isoforms (e.g., pSer239-VASP: total VASP, and/or pSer239-VASP: pSer157-VASP, and/or pSer157-VASP:total VASP). Prognosis is indicated by degree of reduction in the epithelial tumor, whereby the greater the degree of reduction, the more negative the prognosis. For example, Table 1 provides relative ranges of the degree of reduction and the prognosis of a subject.

Progression of a tumor from malignant to invasive, is further indicative of a likelihood of metastasis of the tumor. As such, the methods described herein further relate to a method of determining the likelihood of metastasis of an epithelial tumor in a subject. The method involves determining the degree of reduction of the amount of the different VASP phosphoisomers and of total VASP, independently, or in combination, and/or determining the degree of reduction in one or more relative amounts of the VASP isoforms (e.g., pSer239-VASP: total VASP, and/or pSer239-VASP: pSer157-VASP, and/or pSer157-VASP:total VASP). A greater degree of reduction indicates a higher likelihood of metastasis of the tumor. For example, Table 1 provides relative ranges of the degree of reduction and the likelihood of metastasis of the tumor.

Other aspects of the invention relate to early screening of asymptomatic subjects for epithelial cancer by analysis of levels of pSer157-VASP, pSer239-VASP, and total VASP. Such screening can be performed, for example in similar groups as colonoscopy for screening of colon cancer.

One aspect of the invention relates to a method for diagnosing whether a subject has increased likelihood of developing an epithelial neoplasm such as a tumor or cancer, the method comprising determining the level of pSer157-VASP, pSer239-VASP, and total VASP in the subject, in one or more epithelial tissues of the subject, or in one or more biological samples obtained from the subject, and comparing the level of pSer157-VASP, pSer239-VASP, and total VASP to that determined in an appropriate reference sample, wherein a detectable reduction (e.g., less than 35%) in the level of Ser157-VASP, pSer239-VASP, and/or total VASP, is indicative of the subject having an increased risk of developing an epithelial neoplasm (e.g., epithelial tumor or cancer).

Another aspect of the invention relates to a method for diagnosing whether a subject has an increased likelihood of having an epithelial neoplasm (e.g., epithelial tumor or cancer), the method comprising determining the level of pSer157-VASP, pSer239-VASP, and total VASP in the subject, in epithelial tissue of the subject, or in a biological sample obtained from the subject, and comparing to the level of pSer157-VASP, pSer239-VASP, and total VASP to that determined in an appropriate reference sample, wherein a reduction of 35% or more in the level of pSer157-VASP, pSer239-VASP, and/or total VASP is indicative of the subject having an increased risk of having epithelial cancer at the time the method is performed.

When the subject is identified to be at risk of developing epithelial neoplasm using the methods as disclosed herein, the subject may develop the epithelial neoplasm in the near future or anytime in the future. Accordingly, such subjects can be selected for frequent follow up measurements of the levels of pSer157-VASP, pSer239-VASP, and total VASP to allow early treatment of epithelial neoplasm. Alternatively, the present invention provides methods to diagnose subjects who are at a lesser risk of developing epithelial neoplasm by analyzing the levels of Ser157-VASP, pSer239-VASP, and total VASP to identify subjects at no or minimal risk of epithelial neoplasm, which can be selected to undergo less frequent follow up measurements of the levels of pSer157-VASP, pSer239-VASP, and total VASP, or other alternative invasive diagnostic methods.

The methods described herein are useful for minimally invasive sample procurement and methods for diagnosis and/or prognosis of epithelial neoplasm in a subject, by analyzing the level of pSer157-VASP, pSer239-VASP, and total VASP as disclosed herein in a biological sample from the subject. These methods can be used to diagnosis subjects who are already affected with epithelial neoplasm, or are at high risk of developing epithelial neoplasm. In some embodiments, the subject may be exhibiting a sign or symptom of epithelial neoplasm. In some embodiments, the subject may be asymptomatic or not exhibit a sign or symptom of epithelial neoplasm, but can be at risk of developing epithelial cancer to due to tobacco, alcohol, obesity or other risk factors as described herein.

It will be appreciated that the methods described herein that involve comparison of a determined level or amount to a reference or control amount, that standard methods available to the skilled practitioner can be used to obtain the appropriate reference amounts. In one embodiment, the reference amount is obtained from a reference or control sample obtained from the subject (e.g. a biological sample). The amount is determined by methods identical or analogous to those used to determine the amount of pSer157-VASP. pSer239-VASP, and/or total VASP in the test sample. Typically, normal, healthy tissue, (e.g., located adjacent to the sample tissue), is used as the reference or control tissue. Preferably the control tissue, or biological sample, is of the same type as the tested sample tissue. In certain embodiments, wherein the progression of epithelial cancer in a subject is to be monitored over time, the reference or control sample can be the tested sample tissue taken from the subject at an earlier date.

In some embodiments, the reference can be a level of pSer157-VASP, pSer239-VASP, and total VASP in a normal healthy subject with no symptoms or signs of epithelial neoplasm. Preferably the reference is of the same tissue type as the tested sample. For example, a normal healthy subject has normal epithelial tissue morphology, and/or is not diagnosed with epithelial cancer, and/or has been identified as having a genetic predisposition for developing an epithelial cancer (e.g. a family history of epithelial cancer or a genetic test for genotypes associated with an increased risk of an epithelial cancer) and/or has been exposed to risk factors for epithelial cancers (e.g. smoke, cigarette smoke, alcohol, tobacco, obesity, etc). In some embodiments, the reference can also be a level of pSer157-VASP, pSer239-VASP, and total VASP in a control sample, a pooled sample of control individuals or a numeric value or range of values based on the same.

In some embodiments, the diagnostic methods described herein further comprise the detecting (e.g., the presence or absence, or measuring the level) at least one additional epithelial tumor biomarker known in the art. Useful biomarkers include, without limitation, matrix metalloproteinase 9 (MMP-9; NCBI Gene ID No: 4318), carcinoembryonic antigen (CEA), a biomarker for solid tumors; CA125 (which can be recognized by the monoclonal antibody OC125) a marker for epithelial cancers such as colon or ovarian cancer; and CA19-9, a marker for colon cancer. In one embodiment, the additional biomarker is specific for colorectal cancer development.

Also described herein are methods of enhancing clinical trials comprising choosing appropriate patient populations for those clinical trials. In one aspect, the methods can be used to ensure patients are identified to participate in clinical trials based upon the likelihood of metastasis of the tumor and whether subject can benefit from treatment to prevent the development of cancer.

In one aspect, the methods can comprise measuring an initial value of pSer157-VASP, pSer239-VASP, and total VASP in a subject before the subject has received treatment. Repeat measurements can then be made over a period of time. For example, and not to be limiting, that period of time can be about 1 day, 2 days, 5 days, 1 week, 2 weeks, 3 weeks, 1 month, 2 months, 3 months, 4 months, 6 months, 9 months, 1 year, or greater than 1 year. If the initial level is elevated relative to the mean level in a control population, a significant reduction in level in subsequent measurements can indicate a positive treatment outcome. Likewise, if the initial level of a measure marker is reduced relative to the mean in a control population, a significant increase in measured levels relative to the initial level can signal a positive treatment outcome. Subsequently measured levels are considered to have changed significantly relative to initial levels if a subsequent measured level differs by more than one standard deviation from the mean of repeat measurements of the initial level. If monitoring reveals a positive treatment outcome, that indicates a patient that can be chosen to participate in a clinical trial for that particular therapeutic agent or agents. If monitoring reveals a negative treatment outcome, that indicates a patient that should not be chosen to participate in a clinical trial for that particular therapeutic agent or agents.

### Biological Samples

Provided herein are methods, assays and systems for determining the diagnosis of a subject or the prognosis of a subject by measuring the amount of pSer157-VASP, pSer239-VASP, and VASP in an epithelial tissue (e.g., tumor or suspected tumor tissue) of the subject. Such a measurement can be made, for example, on a representative tissue sample or other form of biological sample, obtained from the subject. In one embodiment, the epithelial cell or tissue is obtained from the subject in the form of a biological sample. The appropriate measurement of pSer157-VASP, pSer239-VASP and/or total VASP is then performed on the biological sample. When necessary, the biological sample can be further processed. For example, a bodily fluid obtained from the subject can be further processed to purify or further concentrate cells or tissues therein.

The term “biological sample” as used herein denotes a sample taken or isolated from a biological organism, e.g., epithelial biopsy sample, tissue cell culture supernatant, cell lysate, lymph, lymph node tissue, a homogenate of a tissue sample from a subject or a fluid sample from a subject. Exemplary biological samples include, but are not limited to, epithelial tissue biopsies, the external sections of the epithelium, epithelial cells, etc. In some embodiments, the sample is normal mucosa, or a polyp.

In one embodiment, the epithelial tissue is an epithelial tumor. In some embodiments the tumor is a primary tumor. In some embodiments the tumor is a secondary tumor. Circulating tumor cells can be further isolated from a biological sample (e.g., bodily fluid) such as blood, lymph, biological fluids, or lymph nodes.

The term “biological sample” also includes untreated or pretreated (or pre-processed) biological samples. In some embodiments, the biological sample is an untreated biological sample. As used herein, the phrase “untreated biological sample” refers to a biological sample that has not had any prior sample pre-treatment except for dilution and/or suspension in a solution. Exemplary methods for treating a biological sample include, but are not limited to, centrifugation, filtration, sonication, homogenization, heating, freezing and thawing, and any combinations thereof. The skilled practitioner is aware of methods and processes appropriate for pre-processing of biological samples required for determination of levels of proteins as described herein.

A biological sample can contain cells from subject. In one embodiment, the biological sample contains non-cellular biological material, such as non-cellular fractions that can be used to measure the levels of pSer157-VASP, pSer239-VASP and/or total VASP. In some embodiments, the sample is from a resection, biopsy, or core needle biopsy. In addition, fine needle aspirate samples can be used. Samples can be either paraffin-embedded or frozen tissue.

The sample can be obtained by removing a sample of cells from a subject, but can also be accomplished by using previously isolated cells (e.g. isolated at a prior timepoint and isolated by the same or another person). In addition, the biological sample can be freshly collected or a previously collected sample. In some embodiments, a biological sample is a biological fluid. Examples of biological fluids include, but are not limited to, saliva, blood, sputum, an aspirate, and any combinations thereof.

In some embodiments, the biological sample is a frozen biological sample, e.g., a frozen tissue or fluid sample such as sputum. The frozen sample can be thawed before employing methods, assays and systems of the invention. After thawing, a frozen sample can be centrifuged before being subjected to methods, assays and systems of the invention. In some embodiments, the biological sample can be treated with at least one chemical reagent, such as a protease inhibitor. In some embodiments, the biological sample is a clarified biological sample, for example, by centrifugation and collection of a supernatant comprising the clarified biological sample. In some embodiments, a biological sample is a pre-processed biological sample, for example, supernatant or filtrate resulting from a treatment selected from the group consisting of centrifugation, filtration, sonication, homogenization, lysis, thawing, amplification, purification, restriction enzyme digestion ligation and any combinations thereof.

In some embodiments, the biological sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. In addition, or alternatively, chemical and/or biological reagents can be employed to release nucleic acid or protein from the sample.

Detection of Levels of pSer157-VASP, pSer239-VASP and/or Total VASP

In one embodiment, the amount of pSer157-VASP, pSer239-VASP, and total VASP in a tissue is determined as the percentage of pSer157-VASP, pSer239-VASP, and total VASP determined for a particular tissue or sample. In one embodiment, the amount of pSer239-VASP, pSer157-VASP, and/or total VASP is measured by quantitation of percentage of epithelial cell compartments with complete loss of pSer239-VASP, pSer157-VASP, and/or total VASP in the epithelial tissue sample. In one embodiment, the amount of pSer239-VASP, pSer157-VASP, and/or total VASP in a tissue sample is measured by quantitation of intensity level detected for pSer239-VASP, pSer157-VASP, and/or total VASP multiplied by the percentage of epithelial cell compartments with that intensity level for the pSer239-VASP, pSer157-VASP, and/or total VASP, respectively.

Methods of Measuring the Amount of pSer157-VASP, pSer239-VASP, and VASP

Methods to measure pSer157-VASP, pSer239-VASP, and total VASP are well known to a skilled artisan. In one embodiment, methods of measurement involve use of a binding protein (e.g. an antibody) for specific detection (e.g., immunodetection) of pSer157-VASP. pSer239-VASP and/or total VASP, Such methods to measure protein level include ELISA (enzyme linked immunosorbent assay), western blot, proteomic microarray, immunoprecipitation, immunofluorescence using detection reagents such as an antibody or protein binding agents. Alternatively, a peptide can be detected in a subject by introducing into a subject a labeled anti-peptide antibody and other types of detection agent. For example, the antibody can be labeled with a radioactive marker whose presence and location in the subject is detected by standard imaging techniques. Such antibody-based methods can distinguish between pSer157-VASP, pSer239-VASP and/or total VASP.

Antibodies specific for the various isoforms of VASP and for total VASP are available in the art. Antibodies specific for pSer239-VASP are available from Abeam (Cat. No. sc-101439; Cambridge, Mass.) and Sigma-Aldrich (Cat. No. SAB4300129; St. Louis, Mo.). Antibody specific for pSer157-VASP is available from Santa Cruz Biotechnology (Cat. No. sc-101440; Santa Cruz, Calif.). Antibody specific to human VASP is available from Santa Cruz Biotechnology (Cat. Nos. sc-13975, sc-46668, sc-101818 or sc-1853; Santa Cruz, Calif.). Alternatively, since the amino acid sequences for the marker genes described herein are known and publically available at NCBI website, one of skill in the art can raise their own antibodies against these proteins of interest for the purpose of the invention.

The amino acid sequences of VASP for different species such as human, mouse and rat, are known in the art. For human VASP protein, the NCBI accession number for the amino acid sequence is NP_{—}003361 (SEQ ID NO: 03).

The techniques of immunohistochemistry (“IHC”) and immunocytochemistry (“ICC”) are particularly suitable for use in the methods described herein. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change (e.g., color or light emission), upon encountering the targeted molecules. In some instances, signal amplification can be integrated into the particular protocol, wherein a secondary antibody, that includes the marker stain or marker signal, follows the application of a primary specific antibody. In some embodiments, the level pSer157-VASP, pSer239-VASP and/or total VASP can be determined using a staining intensity scale and immunohistochemistry as described in the Examples herein.

The technique of proteomic microarray analysis can be used in the methods described herein. Proteomic microarrays are based on miniature arrays of Hgands. Proteins in a sample bind to the ligands on the array and are detected and the amount quantified, often using by fluorescent tags (Templin et al. Proteomics 2003 3:2155-2166; MacBeath, Nature Genetics 2002 32:526-532).

In addition, protein levels may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000). In certain embodiments, a gas phase ion spectrophotometer is used.

In other embodiments, laser-desorption/ionization mass spectrometry is used to analyze the level of a protein. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait). In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094. Detection of the presence of AID mRNA or protein will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

### Treatment of Epithelial Neoplasm

Vasodilator-stimulated phosphoprotein (VASP; NCBI Accession No: NP_{—}003361; SEQ ID NO: 3) is believed to control development of protrusive cell structures. Certain aspects of the invention relate to the finding that pSer157-VASP is a tumor promoter, As such, modification of the phosphorylation state of VASP in an epithelial cell affects the neoplastic, as well as the malignant and invasive state of the cell. Aspects of the invention also relate to the finding that the presence of a VASP protein that has an amino acid substitution of Serine at position 157 for an amino acid that is not phosphorylated (e.g., Alanine), in an epithelial tumor cell or tissue reduces the malignancy and invasiveness of the cell/tumor.

As such, one aspect of the invention relates to a method for reducing the neoplastic growth, malignancy and/or invasiveness of an epithelial tumor cell/tissue. The method comprises introducing an effective amount of such a VASP protein (e.g., S157A-VASP), referred to herein as a VASP 157 mutant, into the cell/tissue. Without being bound by theory it is thought that the presence of the VASP 157 mutant in the cell, alters the state of the cell by altering interactions of the endogenous VASP, perhaps by functioning in a dominant negative capacity, (e.g., by altering phosphorylation or de-phosphorylation or access to a substrate), and is thereby inhibitory of malignant and invasive properties of the cell/tissue. As such, the delivery or expression of an effective amount of the VASP 157 mutant (e.g., a S157A-VASP) molecule to a tumor cell/tissue in a subject can be used to treat the tumor in the subject.

In one embodiment, the method comprises decreasing the level of pSer157-VASP in the epithelial tumor cells. In one embodiment, the method comprises introduction of a VASP 157 mutant into the epithelial tumor cells/tissue.

In one embodiment, the cell or tumor exhibits a reduced amount of one or more of pSer157-VASP, pSer239-VASP, and/or total VASP as described herein. A variety of methods are known in the art for delivering a protein to a cell or tissue (e.g., by delivery of the protein or by delivery of a nucleic acid encoding the protein in expressible form). Suitable methods of delivery are described herein.

One aspect of the invention relates to a method of treating an epithelial neoplasm (e.g., a tumor) in a subject. In one embodiment, the method comprises decreasing the level of pSer157-VASP in the epithelial neoplasm in the subject to thereby treat the epithelial neoplasm in the subject. Any means to decrease the level of pSer157-VASP in a cell can be used in the methods of the present invention, including but not limited to, contacting the cell with an agent or entity which decreases the level of pSer157-VASP within the cell. In some embodiments, agents useful in the present invention include, but without limitation, small molecules, nucleic acids, nucleic acid analogues, proteins, protein fragments, aptamers, antibodies etc as discussed herein in more detail. In some embodiments, the level of pSer157-VASP can be decreased in a cell by conventional gene therapy methods, as commonly known by persons of ordinary skill in the art, and discussed herein.

Another aspect of the invention relates to a method of treating an epithelial neoplasm (e.g., a tumor) in a subject, comprising administering to the subject a therapeutically effective amount of an agent comprising a nucleic acid that encodes the VASP 157 mutant (e.g., S157A-VASP) in expressible form, to the subject to thereby deliver the nucleic acid to the neoplastic epithelial cells of the subject. In one embodiment, the method comprises introduction of a VASP 157 mutant into the epithelial tumor cells.

The methods described herein are suitable for treating epithelial neoplasm and/or tumors in a subject, that range from exhibiting minimal uncontrolled growth, to exhibiting strong invasive properties. Epithelial tumors originating in any epithelial tissue in a subject are suitable for such treatment. Examples of such tumor types and tissues are described herein.

In one embodiment, the epithelial neoplasm is an epithelial cancer. Epithelial cancers suitable for treatment by the methods described herein include, without limitation, colorectal cancer, colon cancer, lung cancer, breast cancer, prostate cancer, liver cancer, pancreatic cancer, kidney cancer, ovarian cancer, gastric cancer, oral cancer, esophageal cancer, gastrointestinal cancer, lip cancer, small bowel cancer, stomach cancer, bladder cancer, and cervical cancer and skin cancer.

In one embodiment, a VASP 157 mutant protein is introduced into the epithelial neoplasm. For example, a nucleic acid encoding VASP, where the one or more of the nucleic acids encoding amino acid 157 have been altered to encode an amino acid other than serine can be used according to the methods described herein. Nucleic acids encoding VASP, where the nucleic acids encoding amino acid 157 have been altered to encode an amino acid other than serine are referred to collectively herein as “nucleic acids encoding S157X-VASP.” In some embodiments, a nucleic acid encoding S157X-VASP comprises at least the coding sequence of VASP (nucleic acids 343-1485 of SEQ ID NO: 04) with at least one change in the nucleic acids 811-813 of SEQ ID NO: 04 such that the endogenous codon “TCC”, which specifies a serine residue in the resulting peptide is changed to a codon specifying a non-serine residue. In some embodiments, the sequence of a nucleic acid encoding S157X-VASP corresponding to nucleic acids 811-813 of SEQ ID NO: 04 encode an alanine residue (i.e. they are “GCT”, “GCC”, “GCA”, or “GCG”). In some embodiments, the nucleic acid encoding S157X-VASP encodes S157A-VASP (SEQ ID NO: 2) and is the nucleic acid of SEQ ID NO: 1. In some embodiments, a nucleic acid encoding S157X-VASP comprises at least the coding sequence of VASP (nucleic acids 343-1485 of SEQ ID NO: 04) with an appropriate mutation. In some embodiments, a nucleic acid encoding S157X-VASP comprises the mRNA of VASP (i.e. SEQ ID NO: 04) with an appropriate mutation. In some embodiments, a nucleic acid encoding S157X-VASP comprises the 5′ and/or 3′ regulatory sequences (e.g. promoters, enhancers, etc) of the endogenous VASP gene. In some embodiments, a nucleic acid encoding S157X-VASP is contained within a vector.

Accordingly, the present invention also encompasses gene therapy methods to introduce a nucleic acid encoding S157X-VASP into a cell, such as an epithelial cell, for lowering the level of pSer157-VASP in the cell. In some embodiments, a nucleic acid encoding pSer157-VASP is introduced into an epithelial cell as disclosed in U.S. Provisional applications 61/185,752 or 61/256,960 which are incorporated herein in their entirety by reference. In some embodiments, a nucleic acid encoding S157X-VASP is introduced into an epithelial cell as disclosed in International Applications WO/2008/054819 and WO/2009/114673, which are incorporated herein by reference.

In some embodiments, the agent comprises a nucleic acid which encodes a polypeptide having at least 80% sequence identity to SEQ ID NO: 2, or alternatively at least 85%, or at least 90%, or at least 95%, or at least 98%, or at least 99%, or at least 100% sequence identity to SEQ ID NO: 2. In one embodiment, the polypeptide has the sequence recited in SEQ ID NO: 2, with one or more conservative amino acid substitutions therein. In some embodiments, the agent comprises a fragment, derivative, or functional fragment of S157X-VASP or a nucleic acid encoding S157X-VASP. In alternative embodiments, the agent is any agent which inhibits phosphorylation of Ser157 of VASP.

A nucleic acid sequence encoding S157X-VASP can be prepared by recombinant techniques commonly known by one of ordinary skill in the art. In some embodiments, derivatives and functional fragments of S157X-VASP can be prepared by techniques commonly known by one of ordinary skill in the art, such as deletions, additions, conservative amino acid substitution, or other manipulations that produce a molecule that maintains the property of tumor suppression as indicated by the methods exemplified herein.

It is well known by persons of ordinary skill in the art that derivatives of natural proteins often retain a biological activity similar to that of the naturally occurring protein. For example, Gayle and coworkers conducted extensive mutational analysis of human cytokine IL-1a. Gayle et al. J. Biol. Chem. 268: 22105-22111 (1993). They used random mutagenesis to generate over 3,500 individual IL-1α mutants that averaged 2.5 amino acid changes per variant over the entire length of the molecule. Multiple mutations were examined at every possible amino acid position. The investigators found that most of the molecule could be altered with little effect on either binding or biological activity. In fact, only, 23 unique amino acid sequences, out of more than 3,500 nucleotide sequences examined, produced a protein that significantly differed in activity from that of the wild-type.

Guidance concerning how to make phenotypically silent amino acid substitutions is provided in Bowie et al., “Deciphering the Message in Protein Sequences: Tolerance to Amino Acid Substitutions,” Science 247: 1306-1310 (1990). The authors indicate that there are two main strategies for studying the tolerance of an amino acid sequence to change. The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection are likely positions that are not critical for protein function. Thus, positions that tolerate amino acid substitution could be modified while still maintaining biological activity of the protein.

The second strategy uses genetic engineering to introduce amino acid changes at specific positions of a cloned gene to identify regions critical for protein function. For example, site directed mutagenesis by alanine-scanning mutagenesis (introduction of single alanine mutations at every residue in the molecule) may be used. Cunningham and Wells, Science 244: 1081-1085 (1989). Bowie et al., Science 247: 1306-1310 (1990), indicate that these two strategies have shown that proteins are surprisingly tolerant of amino acid substitutions. The authors further disclose which amino acid changes are likely to be permissive at certain amino acid positions in the protein. For example, most buried (within the tertiary structure of the protein) amino acid residues require nonpolar side chains, whereas few features of surface side chains are generally conserved.

In general, amino acid substitutions made in order to prepare derivatives or variants of S157X-VASP polypeptide are accomplished by selecting substitutions that do not differ significantly in their effect on maintaining, for example, (a) the structure of the nuclear form of S157X-VASP peptide backbone in the area of the substitution, (b) the charge or hydrophobicity of S157X-VASP polypeptide at the target site, or (c) the bulk of a side chain. Naturally occurring residues are divided into groups based on common side-chain properties, for example: (1) hydrophobic: norleucine, met, ala, val, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic; trp, tyr, phe. Therefore, conservative amino acid substitutions include, for example, substitution of an aspartic acid residue by a glutamic acid residue because both are acidic amino acids. Similarly, the following examples show acceptable conservative substitutions: lysine/arginine/histidine as basic amino acids; leucine/isoleucine, methionine/valine, alanine/valine as hydrophobic amino acids; serine/glycine/alanine/threonine as hydrophilic amino acids.

Conservative amino acid substitutions also include groupings based on side chains. For example, substitutions include substitutions among amino acids that belong to a group having aliphatic side chains, such as glycine, alanine, valine, leucine, and isoleucine. Similarly, substitutions among a group of amino acids having aliphatic-hydroxyl side chains include substitutions between serine and threonine; substitutions among a group of amino acids of amide-containing side chains include substitutions between asparagine and glutamine; substitutions among a group of amino acids having aromatic side chains include substitutions between phenylalanine, tyrosine, and tryptophan; substitutions among a group of amino acids having basic side chains include substitutions between lysine, arginine, and histidine; and substitutions among a group of amino acids having sulfur-containing side chains include substitutions between cysteine and methionine. Therefore, one skilled in the art can predict based on these guidelines that replacement of a leucine with an isoleucine or valine, an aspartate with a glutamate, a threonine with a serine, or a similar replacement of an amino acid with a structurally related amino acid will not have a major effect on the properties of the resulting S157X-VASP polypeptide derivative.

Additionally, computer programs that predict the effect of a specific amino acid substitution or mutation on the structure of the protein are available. PCT Patent Application No. WO 02/543063 (which is incorporated in its entirety herein by reference) discloses one such method and also reviews the state of the art in this field. Whether any amino acid change (deletion, addition, substitution, or a combination thereof) results in a functional S157X-VASP polypeptide derivative can readily be determined by assaying for suppression of epithelial tumor metastasis as described in the examples or alternatively, by assaying the level of pSer157-VASP in a cell or a subject as disclosed herein.

In addition to conservative amino acid substitutions, functional S157X-VASP polypeptide derivatives used in the instant invention include (i) substitutions with one or more non-conserved amino acid residues, where the substituted amino acid residue may be a chemically modified amino acid (e.g., by methylation, acylation, etc.) that can or can not be encoded by the genetic code, (ii) substitutions with one or more amino acid residues having a substituent group, (iii) fusion of S157X-VASP polypeptide with another compound, such as a compound to increase the stability and/or solubility of S157X-VASP polypeptide (for example, polyethylene glycol), or (iv) fusion of S157X-VASP polypeptide with additional amino acids. Examples of preparation of derivatives following these guidelines can be found in, for example, U.S. Pat. No. 5,876,969, EP Patent 0 413 622, and U.S. Pat. No. 5,766,883, which are incorporated herein in their entirety by reference.

Such functional derivatives of S157X-VASP polypeptides may be administered directly to a cell or subject instead of administering a nucleic acid encoding for a functional derivative of S157X-VASP.

Functional derivatives of S157X-VASP polypeptide that contain deletions or additions of amino acid residues may be produced by using known methods of protein engineering and recombinant DNA technology. For instance, one or more amino acids can be deleted or added from either the N-terminus or C-terminus of S157X-VASP without substantial loss of biological function as compared to the polypeptide corresponding to SEQ ID NO: 02. For example, variant keratinocyte growth factor (KGF) proteins having heparin binding activity even after deleting 3, 8, or 27 amino-terminal amino acid residues. Ron et al. J. Biol. Chem. 268: 2984-2988 (1993). Similarly, interferon gamma exhibited up to ten times higher activity after deleting 8-10 amino acid residues from the carboxy terminus of the protein. Dobeli et al., J. Biotechnol. 7: 199-216 (1988).

Accordingly, functional derivatives of the S157X-VASP may be prepared by modification of the amino acids encoded by S157X-VASP (i.e. SEQ ID NO: 2) or in a derivative of S157X-VASP. Modifications may occur anywhere in the encoded VASP 157 mutant polypeptide sequence or its functional derivative polypeptide, including the peptide backbone, the amino acid side-chains and the amino or carboxyl termini. Modifications may include, for example, acetylation, acylation, ADP-ribosylation, amidation, covalent attachment of other functional moiety, covalent attachment of a lipid or lipid derivative, covalent attachment of phosphotidylinositol, cross-linking, cyclization, disulfide bond formation, demethylation, formation of covalent cross-links, formylation, gamma-carboxylation, glycosylation, glycophosphatidylinositol (GPI) anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, E. Creighton Proteins-Structure and Molecular Properties, 2nd Ed., W. H. Freeman and Company, New York (1993); B. C. Johnson, Post Translational Covalent Modification of Proteins, Academic Press, New York, (1983); Seifter et al., Meth. Enzymol. 182: 626-646 (1990); Rattan et al., Ann. N.Y. Acad. Sci. 663: 48-62 (1992). Preparation of these modified derivatives may, for example, be useful if direct administration of the derivative, rather than administration of a nucleic acid encoding the derivative, is contemplated.

Use of natural and non-natural allelic variations of the VASP molecule for administration is also contemplated as being part of the invention. These allelic variants can vary at either the polynucleotide and/or polypeptide level. Non-naturally occurring variants may be produced by mutagenesis techniques or by direct synthesis. Therefore, the present invention is also directed to proteins containing polypeptides at least 80%, 85%, 90%, 95%, 96%, 97%, 98% or 99% identical to VASP polypeptide sequences corresponding to SEQ ID NO: 2. The methods of the invention may also utilize nucleic acid molecules comprising, or alternatively, consisting of, a nucleotide sequence at least 70%, 75%, 80%, 85%, 90%, 95%, 96%, 97%, 98% or 99% identical to the nucleotide sequence encoding VASP. Whether any particular nucleic acid molecule or polypeptide is at least 80%, 85%, 90%, 95%, 96%, 97%, 98% or 99% identical to VASP sequence, or a portion of a nucleic acid sequence encoding VASP can be determined conventionally using known computer programs. See, e.g., WO 01/54474 which is incorporated herein by reference.

One method for decreasing the level of pSer157-VASP in a cell is the inhibition of phosphorylation of the Ser157 of the VASP protein, or the induction of dephosphorylation of the pSer157 of the VASP protein.

### S157X-VASP Gene Therapy Nucleic Acids

In one embodiment, treatment can include contacting a cell with a single stranded nucleic acids encoding S157X-VASP or a fragment thereof, in order to direct one or more changes in the endogenous VASP mRNA that will result in S157X-VASP being transcribed instead of wild-type VASP. Such methods are well known to those skilled in the art and include, but are not limited to Spliceosome-Mediated RNA Trans-splicing (SMaRT; Dallinger et al., Experimental Dermatology 2003 12:37-46). In one embodiment, a single base change is introduced into the endogenous VASP mRNA. In one embodiment, multiple base changes are introduced into the endogenous VASP mRNA. In some embodiments, the single stranded nucleic acid is delivered to the cell as naked nucleic acid (i.e. not in a vector). In one embodiment, the single-stranded nucleic acid is delivered to the cell by a nucleic acid which encodes the single-stranded nucleic acid contained within a vector as described elsewhere herein.

In one embodiment, the nucleic acid that encodes the VASP 157 mutant is within a viral vector that drives the expression of the encoded polypeptides in infected host cells. Viral vectors are well known to those skilled in the art and discussed further herein below. Other avenues for the administration of S157X-VASP polypeptides for therapy are discussed herein below.

In some embodiments, where the agent is a nucleic acid encoding S157X-VASP or a variant or mutant thereof, the agent or can also be a fusion polypeptide, fused, for example, to a polypeptide that targets the product to a desired location, or, for example, a tag that facilitates its purification, if so desired. Fusion to a polypeptide sequence that increases the stability of the agent is also contemplated. For example, fusion to a serum protein, e.g., serum albumin, can increase the circulating half-life of the agent. Tags and fusion partners can be designed to be cleavable, if so desired. Another modification specifically contemplated is attachment, e.g., covalent attachment, to a polymer. In one aspect, polymers such as polyethylene glycol (PEG) or methoxypolyethylene glycol (mPEG) can increase the in vivo half-life of proteins to which they are conjugated. Methods of PEGylation of polypeptide agents are well known to those skilled in the art, as are considerations of, for example, how large a PEG polymer to use. Fusion to serum albumin can also increase the serum half-life of therapeutic polypeptides.

### S157X-VASP Protein

In some embodiments, the VASP 157 mutant protein described herein is administered to the subject, to thereby be delivered to the epithelial tumor cells of the subject. In some embodiments, preparation of a S157X-VASP polypeptide, for use in the methods and compositions as disclosed herein can be produced by any means known by one of ordinary skill in the art, for example in cultured cells expressing the recombinant S157X-VASP protein. In some embodiments, the S157X-VASP polypeptide or a functional portion or fragment thereof can be administered by any means known by a skilled artisan, for example direct administration of the protein, or administration of cells expressing the recombinant S157X-VASP protein.

Administration can be via any route, for example but not limited to administration intravenously. In alternative embodiments, the polypeptide can be administered directly to location of the epithelial tissue to be treated by any means, or other routes of therapeutic protein administration are contemplated such as inhalation. Technologies for the administration of agents, including protein agents, as aerosols are well known and continue to advance. Alternatively, the polypeptide agent can be formulated in liposomes for topical delivery. Further contemplated are, for example, transdermal administration, and rectal or vaginal administration. Further options for the delivery of S157X-VASP polypeptides as described elsewhere herein. Dosage ranges will vary, depending upon the individual, the degree of disease severity, and the specific polypeptide administered, but can be readily selected and adjusted by the administering clinician. An exemplary dose range is approximately 0.01 μg/kg to 1 mg/kg per dose, with doses administered, for example, once a week, once every three days, once every other day, or even daily. Initial doses can be greater, to establish an effect, and then reduced to a maintenance level thereafter.

In some embodiments, the heterologous promoter allows controlled expression of S157X-VASP, such as for example, a drug or stress inducible promoter, such as a Tet-inducible system and the like. For example, cells can be engineered to express S157X-VASP under the control of an endogenous promoter, or S157X-VASP under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al., which are incorporated herein in their entirety by reference.

### Antibodies

In some embodiments, the level of pSer157-VASP is reduced in a cell by contacting the cell with an agent which decreases the level of pSer157-VASP. In one embodiment, the agent is an antibody. In one embodiment, the antibody specifically binds pSer157-VASP, including monoclonal, chimeric humanized and recombinant antibodies and antigen-binding fragments thereof. In one embodiment, the agent is an antibody which binds to and inhibits a protein, which when not bound by the antibody, functions to promote the phosphorylation of pSer157 of VASP.

Antibodies useful in the present invention can readily raised in animals such as rabbits or mice by immunization with the antigen. Immunized mice are particularly useful for providing sources of B cells for the manufacture of hybridomas, which in turn are cultured to produce large quantities of monoclonal antibodies. In some embodiments of this invention, an agent which specifically binds pSer157-VASP, pSer239-VASP, or total VASP as described herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. Murine monoclonal antibodies can preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

### Delivery of Nucleic Acids

Methods of delivering nucleic acid agents, e.g., a nucleic acid encoding S157X-VASP or vectors containing nucleic acid encoding S157X-VASP, to a target cell (e.g., epithelial tumor cell or other desired target cells), can include, for example (i) injection of a composition containing the nucleic acid agent, or (ii) directly contacting the cell with a composition comprising an nucleic acid agent. In another embodiment, nucleic acids can be injected directly into any blood vessel, such as vein, artery, venule or arteriole, via, e.g., hydrodynamic injection or catheterization. In some embodiments nucleic acid agents can delivered to specific organs or tissues, or systemically administered.

Specific epithelial cells (e.g., specific epithelial tumor cells) can be targeted by the nucleic acid agents. The method can use, for example, a complex or a fusion molecule comprising a cell targeting moiety and a nucleic acid binding moiety that is used to deliver nucleic acids effectively into cells. For example, an antibody-protamine fusion protein when mixed with a nucleic acid agent binds the nucleic acid and selectively delivers the nucleic acid agent into cells expressing an antigen recognized by the antibody, resulting in silencing of gene expression only in those cells that express the antigen. The nucleic acid molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

A viral-mediated delivery mechanism can also be employed to deliver nucleic acids to cells as described herein.

### Vectors and Expression of Agents

As discussed herein, in some embodiments, a nucleic acid encoding S157X-VASP is introduced into a cell and the S157X-VASP polypeptide expressed. Any means to introduce the nucleic acid into the cell can be used, for example by using gene therapy techniques such as use of an expression vector, or viral vector. In some embodiments, the nucleic acid can be introduced as naked DNA and by standard transfection methods. One method of expression of a nucleic acid in a cell is by insertion of the nucleic acid into an expression vector.

Expression vectors compatible with eukaryotic cells, preferably those compatible with vertebrate cells, can be used to produce recombinant constructs useful as agents described herein. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus (AAV), pox virus such as an orthopox (vaccinia and attenuated vaccinia), avipox, murine moloney leukemia virus, retrovirus (e.g. gammaretrovirus or lentivirus), or pseudotype virus etc. Alternatively, plasmid expression vectors can also be used.

Viral vector systems which can be utilized in the present invention include, but are not limited to, (a) adenovirus vectors; (b) retrovirus vectors; (c) adeno-associated virus vectors (AAV); (d) herpes simplex virus vectors (HSV); (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus, EPV and EBV vectors. In some embodiments, the vector is an adenovirus or an adeno-associated virus. In some embodiments, the vector is replication competent. In some embodiments, the vector is replication defective.

Vectors for expression of a nucleic acid encoded therein generally contain regulatory elements, e.g., promoters, enhancers, etc., to ensure the expression of the construct in target cells. Other specifics for vectors and constructs are described in further detail below.

As used herein, a “promoter” or “promoter region” or “promoter element” used interchangeably herein refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “regulatory sequences” is used interchangeably with “regulatory elements” herein refers element to a segment of nucleic acid, typically but not limited to DNA or RNA or analogues thereof, that modulates the transcription of the nucleic acid sequence to which it is operatively linked, and thus act as transcriptional modulators. Regulatory sequences modulate the expression of gene and/or nucleic acid sequence to which they are operatively linked. Regulatory sequence often comprise “regulatory elements” which are nucleic acid sequences that are transcription binding domains and are recognized by the nucleic acid-binding domains of transcriptional proteins and/or transcription factors, repressors or enhancers etc. Typical regulatory sequences include, but are not limited to, transcriptional promoters, inducible promoters and transcriptional elements, an optional operate sequence to control transcription, a sequence encoding suitable mRNA ribosomal binding sites, and sequences to control the termination of transcription and/or translation. Regulatory sequences can be a single regulatory sequence or multiple regulatory sequences, or modified regulatory sequences or fragments thereof. Modified regulatory sequences are regulatory sequences where the nucleic acid sequence has been changed or modified by some means, for example, but not limited to, mutation, methylation etc.

The nucleic acids for use in the therapeutic methods described herein can be delivered to a target cell in expressible form. The term, in expressible form is used to refer to the fact that the nucleic acid contains all of the materials necessary for expression once delivered to the target cell. Typically the nucleic acid is operatively linked to required regulatory sequences. The term “operatively linked” as used herein refers to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined. In some embodiments, it can be advantageous to direct expression of the encoded VASP polypeptide in a tissue- or cell-specific manner. Epithelial-specific expression can be achieved for example, by using the EGP-2 promoter (McLaughlin et al., Cancer Res 2001 61:4105) or other epithelial-specific promoters known in the art. Promoters specific for certain types of epithelial tissue can also be used. By way of non-limiting example, the lung surfactant protein B promoter is specific for lung epithelial tissue (Bohinski et al. Mol Cell Biol 1994 14:5671-5681).

In some embodiments of the invention described herein, viral vectors that contain nucleic acid sequences described herein are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences can be cloned into one or more vectors, which facilitate delivery of the nucleic acid into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

The production of a recombinant retroviral vector carrying a nucleic acid of interest is typically achieved in two stages. First, the desired nucleotide sequences are inserted into a retroviral vector which contains the sequences necessary for the efficient expression of the nucleic acids (including promoter and/or enhancer elements which can be provided by the viral long terminal repeats (LTRs) or by an internal promoter/enhancer and relevant splicing signals), sequences required for the efficient packaging of the viral RNA into infectious virions (e.g., a packaging signal (Psi), a tRNA primer binding site (−PBS), a 3′ regulatory sequence required for reverse transcription (+PBS)), and a viral LTRs). The LTRs contain sequences required for the association of viral genomic RNA, reverse transcriptase and integrase functions, and sequences involved in directing the expression of the genomic RNA to be packaged in viral particles.

Following the construction of the recombinant retroviral vector, the vector DNA is introduced into a packaging cell line. Packaging cell lines provide viral proteins required in trans for the packaging of viral genomic RNA into viral particles having the desired host range (e.g., the viral-encoded core (gag), polymerase (pol) and envelope (env) proteins). The host range is controlled, in part, by the type of envelope gene product expressed on the surface of the viral particle. Packaging cell lines can express ecotrophic, amphotropic or xenotropic envelope gene products. Alternatively, the packaging cell line can lack sequences encoding a viral envelope (env) protein. In this case, the packaging cell line can package the viral genome into particles which lack a membrane-associated protein (e.g., an env protein). To produce viral particles containing a membrane-associated protein which permits entry of the virus into a cell, the packaging cell line containing the retroviral sequences can be transfected with sequences encoding a membrane-associated protein (e.g., the G protein of vesicular stomatitis virus (VSV)). The transfected packaging cell can then produce viral particles which contain the membrane-associated protein expressed by the transfected packaging cell line; these viral particles which contain viral genomic RNA derived from one virus encapsidated by the envelope proteins of another virus are said to be pseudotyped virus particles.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Another preferred viral vector is a pox virus such as a vaccinia virus, for example an attenuated vaccinia such as Modified Virus Ankara (MVA) or NYVAC, an avipox such as fowl pox or canary pox. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In another embodiment, lentiviral vectors are used, such as the HIV based vectors described in U.S. Pat. Nos. 6,143,520; 5,665,557; and 5,981,276, which are herein incorporated by reference. Use of Adeno-associated virus (AAV) vectors is also contemplated (Walsh et al., Proc. Soc. Exp. Biol. Med. 204:289-300 (1993); U.S. Pat. No. 5,436,146).

The ‘gene gun’, or biolistics, is a method of introducing nucleic acids into cells by coating a particle of heavy metal with nucleic acid which is propelled into the cell, often using a pneumatic device.

Polyplex delivery is a method of using polymers to form ionic complexes with nucleic acids. The polyplex can increase the rate of cellular uptake of the nucleic acid, increase stability of the nucleic acid in circulation, and aid in endosomes escape after cellular internalization (Christie et al. Endocrinology, 2010 151:466). Dendrimer delivery utilizes dendrimers, molecules which are repetitively branched and roughly spherical. Dendrimers can be specifically engineered to carry other molecules in the interior of the sphere. A virosome is similar to a liposome but incorporates viral envelope proteins to enhance delivery of the nucleic acid contained within the vesicle.

U.S. Pat. No. 5,676,954 (which is herein incorporated by reference) reports on the injection of genetic material such as naked DNA, complexed with cationic liposome carriers, into mice. U.S. Pat. Nos. 4,897,355, 4,946,787, 5,049,386, 5,459,127, 5,589,466, 5,693,622, 5,580,859, 5,703,055, and international publication NO: WO 94/9469 (which are all herein incorporated by reference) provide cationic lipids for use in transfecting DNA into cells and mammals. U.S. Pat. Nos. 5,589,466, 5,693,622, 5,580,859, 5,703,055, and international publication NO: WO 94/9469 (which are herein all incorporated by reference) provide methods for delivering DNA-cationic lipid complexes to mammals. Such cationic lipid complexes or nanoparticles can also be used to deliver protein.

A nucleic acid sequence can be introduced into a target cell by any suitable method. For example, by transfection (e.g., calcium phosphate or DEAE-dextran mediated transfection), lipofection, electroporation, microinjection (e.g., by direct injection of naked DNA), biolistics, infection with a viral vector containing a muscle related transgene, cell fusion, chromosome-mediated gene transfer, microcell-mediated gene transfer, nuclear transfer, and the like.

Various delivery systems are known and can be used to directly administer therapeutic polypeptides, e.g., encapsulation in liposomes, microparticles, microcapsules, recombinant cells capable of expressing the compound, and receptor-mediated endocytosis (see, e.g., Wu and Wu, 1987, J. Biol. Chem. 262:4429-4432). Methods of introduction can be enteral or parenteral and include but are not limited to intradermal, intramuscular, intraperitoneal, intravenous, subcutaneous, pulmonary, intranasal, intraocular, epidural, and oral routes. The agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, the implant being of a porous, non-porous, or gelatinous material, including membranes, such as sialastic membranes, fibers, or commercial skin substitutes. In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105).

Assessment of Agents which Decrease pSer157-VASP

In some embodiments, the agent which decreases the level of pSer157-VASP useful in the methods and compositions of the present invention can be assessed using the assay for peritoneal colon cancer metastasis as disclosed herein in the Examples. For example, an agent which decreases the level of pSer157-VASP can be identified by one of ordinary skill in the art as an agent which, when introduced into an epithelial cancer cell, decreases the rate of metastasis as compared to a control agent or the absence of an agent. Alternatively, one of ordinary skill in the art can contact a cell with an agent useful in the method described herein and determine the level of pSer157-VASP in the presence or absence of the agent, using immunohistochemistry analysis according to the Examples herein, or other similar protein detection methods. Alternatively, one can perform immunohistochemistry analysis using an anti-pSer157 antibody, and any agent which when added to a cell decreased the level of pSer157 in the presence of the agent as compared to a control agent (or the absence of the agent) would identify that the agent would be useful in the methods and compositions as disclosed herein.

In some embodiments, the agents disclosed herein for the treatment or prevention initiation, progression, or metastasis of an epithelial neoplasm are administered in combination with one or more additional agents. In one embodiment, one or more cGMP agonists and/or cAMP-elevating agents are concurrently administered. Examples of cGMP agonists include, but are not limited to, bacterial enterotoxin ST, guanylin, uroguanylin, zaprinast, sildenafil, and 8br-cGMP, Examples of cAMP-elevating agents include, but are not limited to, forskolin, 8br-cAMP, and prostaglandins.

The dosage ranges for the administration of an agent useful in the methods described herein will depend upon the method by which the agent is delivered, and its potency, as described further herein, and are amounts large enough to produce the desired effect in which the effects of are reduced, for example but not limited to; decreased tumor size, decreased tumor initiation or metastasis phenotypes and/or markers, a decrease in one or more signs, symptoms, or markers of epithelial cancer. The dosage should not be so large as to cause adverse side effects. Generally, the dosage will vary with the age, condition, and sex of the patient and can be determined by one of skill in the art. The dosage can also be adjusted by the individual physician in the event of any complication.

With respect to the therapeutic methods of the invention, it is not intended that the administration of the compositions described herein be limited to a particular mode of administration, dosage, or frequency of dosing; the present invention contemplates all modes of administration, including intramuscular, intravenous, intraperitoneal, intravascular, intraarticular, intralesional, subcutaneous, or any other route sufficient to provide a dose adequate to treat or prevent the progression of epithelial cancer. The therapeutic composition may be administered to the patient in a single dose or in multiple doses. When multiple doses are administered, the doses may be separated from one another by, for example, one hour, three hours, six hours, eight hours, one day, two days, one week, two weeks, or one month. For example, the therapeutic may be administered for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity.

Effective doses may be extrapolated from dose-response curves derived from in vitro or animal model test bioassays or systems.

Dosages for a particular patient or subject can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the agent as disclosed herein, and the condition of the patient, as well as the body weight or surface area of the patient to be treated. Therapeutic compositions comprising agent are optionally tested in one or more appropriate in vitro and/or in vivo animal models of disease

### Amenable Subjects

Certain aspects of the invention described herein relate to administering the compositions described herein to a subject having epithelial cancer or diagnosed as having epithelial cancer or at risk of having epithelial cancer, and/or is in need of treatment for epithelial cancer.

Subjects having epithelial cancer can be identified by a physician using current methods of diagnosing epithelial cancer. Subjects at risk of having or developing epithelial cancer include subjects who have smoked tobacco or been exposed to tobacco smoke. Additional factors which increase the likelihood of a subject developing an epithelial cancer include, but are not limited to, alcohol, obesity, or a family history of epithelial cancer.

In some embodiments, the pharmaceutical composition is used to treat epithelial cancer. In some embodiments, the subject is selected for having an epithelial cancer before being administered the composition. In some embodiments, the pharmaceutical composition further comprises additional agents to treat epithelial cancer.

### Pharmaceutical Formulations

A pharmaceutical composition as described herein comprises an agent and a pharmaceutically acceptable carrier or excipient. Excipients useful for preparing the dosages forms from the composition according to the invention and the instruments necessary to prepare them are described in U.S. Publication No.: 2003/0206954 and 2004/0052843, which are incorporated herein in their entirety by reference.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the agent which decreases the level of pSer157-VASP.

As described in detail below, the pharmaceutical compositions can be specially formulated for administration to a subject in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, the composition can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

### Combination Therapies

As disclosed herein, the compositions described herein can be administrated to a subject alone, or optionally in combination (e.g. simultaneously with, sequentially or separately) with one or more additional pharmaceutically active agents, e.g. a second therapeutic agent known to be beneficial in treating epithelial cancer. For example, exemplary pharmaceutically active compound include, but are not limited to, those found in Harrison's principals of Internal Medicine, 13th Edition, Eds. T. R. Harrison et al. McGraw-Hill N.Y., N.Y.; Physicians Desk Reference, 50^{th} Edition, 1997, Oradell N.J., Medical Economics Co.; Pharmacological Basis of Therapeutics, 8th Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

Non-limiting examples of chemotherapeutic agents are paclitaxel, cisplatin, doxorubicin, rapamycin. Also included as chemotherapeutic agents in the pharmaceutical compositions of this invention are nitrogen mustards such as cyclophosphamide, ifosfamide, and melphalan; ethylenimines and methylmelamines such as hexamethylmelamine and thiotepa; pyrimidine analogs such as fluorouracil and fluorodeoxyuridine; vinca alkaloids such as vinblastine; epipodophyllotoxins such as etoposide and teniposide; antibiotics such as actinomycin D, doxorubicin, bleomycin, and mithramycin; biological response modifiers such as interferon; platinum coordination complexes such as cisplatin and carboplatin; estrogens such as diethylstilbestrol and ethinyl estradiol; antiandrogens such as flutamine; and gonadotropin releasing hormone analogs such as leuprolide. Other compounds such as decarbazine, nitrosoureas, methotrexate, diticene, and procarbazine are also effective. Of course, other chemotherapeutic agents which are known to those of ordinary skill in the art can readily be substituted as this list should not be considered exhaustive or limiting.

Systems for Identifying a Subject with Increased Risk for Having Epithelial Cancer or Needing Treatment for Epithelial Cancer

In another embodiment of the assays described herein, the assay comprises or consists essentially of a system for quantitatively transforming an entity (e.g., pSer157-VASP, pSer239-VASP, and total VASP) in a biological sample, into a detectable target, and measuring the amount of the detectable target, as quantitatively representing the respective amounts of pSer157-VASP, pSer239-VASP, and/or total VASP in the biological sample from which they were obtained, as described herein. The assays further comprise comparing the amount, or the relative amounts (discussed herein) of the entities, to a similarly obtained reference level. Reference levels can be obtained, for example, by measuring the amount of the appropriate detectable target in an appropriate control sample. If the comparison system, which can be a computer implemented system, indicates that the amount of the entity, or the relative amount of one or more of the entities is statistically different (reduced) from that of the reference amount, the subject from which the biological sample is collected can be identified as having one or more of the conditions described herein.

Embodiments of the invention also provide for systems (and computer readable media for causing computer systems) to perform the various methods described herein by measuring the level of pSer157-VASP, pSer239-VASP, and/or total VASP and comparison to an appropriate reference level.

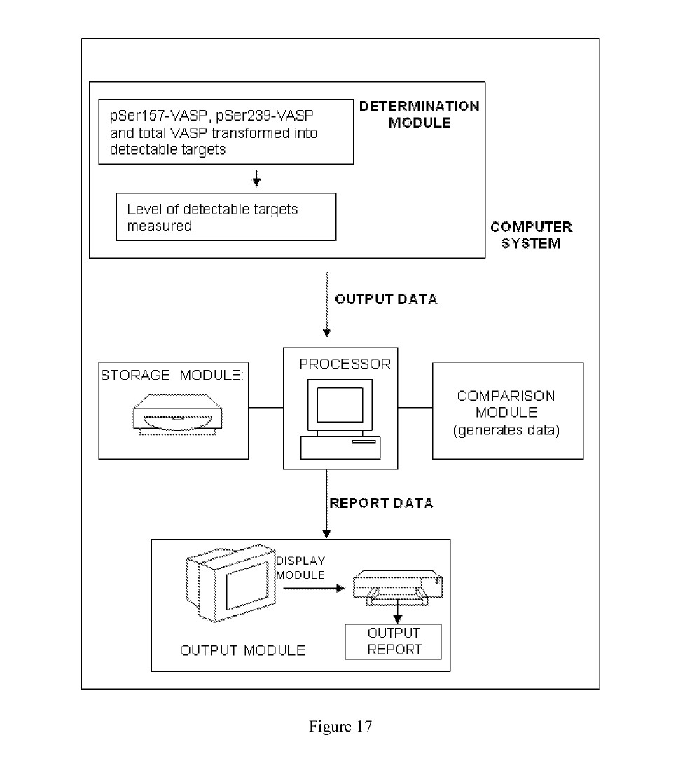

In one embodiment, provided herein is a system comprising: (a) at least one memory containing at least one computer program adapted to control the operation of the computer system to implement a method that includes (i) a determination module configured to identify and detect at the level of pSer157-VASP, pSer239-VASP, and/or total VASP, in a biological sample obtained from a subject; (ii) a storage module configured to store output data from the determination module; (iii) a computing module adapted to identify from the output data whether the level of pSer157-VASP, pSer239-VASP, and/or total VASP, in the biological sample obtained from a subject is lower by a statistically significant amount from the level found in a reference sample and (iv) a display module for displaying whether pSer157-VASP, pSer239-VASP, and/or total VASP, are present at a statistically significant lower level in the tissue sample obtained from a subject as compared to the reference level and/or displaying the relative levels of pSer157-VASP, pSer239-VASP, and/or total VASP, (b) at least one processor for executing the computer program (see FIG. 17).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination module, a storage module, a computing module, and a display module. The functional modules can be executed on one, or multiple, computers, or by using one, or multiple, computer networks. The determination module has computer executable instructions to provide e.g., allelic variance etc in computer readable form.

The determination module can comprise any system for detecting a signal elicited from pSer157-VASP, pSer239-VASP, or total VASP in a biological sample. In some embodiments, such systems can include an instrument, e.g., the Cell Biosciences NanoPro 1000 System (Cell Biosciences) for quantitative measurement of proteins and protein phosphoisoforms. In another embodiment, the determination module can comprise multiple units for different functions, such as detection and hybridization. In one embodiment, the determination module can be configured to perform the reactions and analysis needed to measure the levels of pSer157-VASP, pSer239-VASP, or total VASP, in a sample using an immunohistochemical assay, including hybridization, detection, and analysis.

In some embodiments, the determination module can be further configured to identify and detect the presence of at least one additional epithelial cancer-related biomarker. In some embodiments, the additional epithelial cancer-related biomarker is matrix metalloproteinase 9 (MMP-9; NCBI Gene ID No: 4318).

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, alleleic variants, and frequency of each alleleic variant. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as expression levels of the marker genes described herein in subjects who do not have symptoms associated with emphysema. In certain embodiments, the storage module stores the reference information such as levels of pSer157-VASP, pSer239-VASP, or total VASP, described herein in a sample of healthy epithelial tissue obtained from the subject or in a sample from the subject taken at an earlier time.

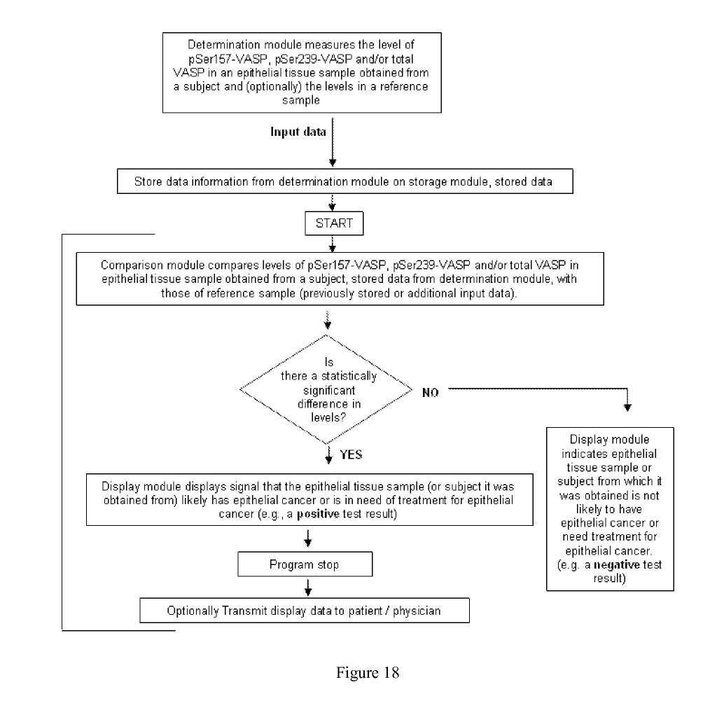

The “computing module” can use a variety of available software programs and formats for computing the relative expression level of the marker genes described herein. Such algorithms are well established in the art. A skilled artisan is readily able to determine the appropriate algorithms based on the size and quality of the sample and type of data. Such data analysis tools can be implemented in the computing module of the invention. In one embodiment, the computing module further comprises a comparison module, which compares the level of pSer157-VASP, pSer239-VASP, or total VASP, in the epithelial tissue sample obtained from a subject as described herein with the reference level of pSer157-VASP, pSer239-VASP, or total VASP, (FIG. 18). By way of an example, when the level of pSer239-VASP in the epithelial tissue sample obtained from a subject is measured, a comparison module can compare or match the output data—with the reference level of pSer239-VASP in a reference sample. In certain embodiments, the reference level can have been pre-stored in the storage module. During the comparison or matching process, the comparison module can determine whether the level in the epithelial tissue sample obtained from a subject is lower than the reference level to a statistically significant degree. In various embodiments, the comparison module can be configured using existing commercially-available or freely-available software for comparison purpose, and may be optimized for particular data comparisons that are conducted.

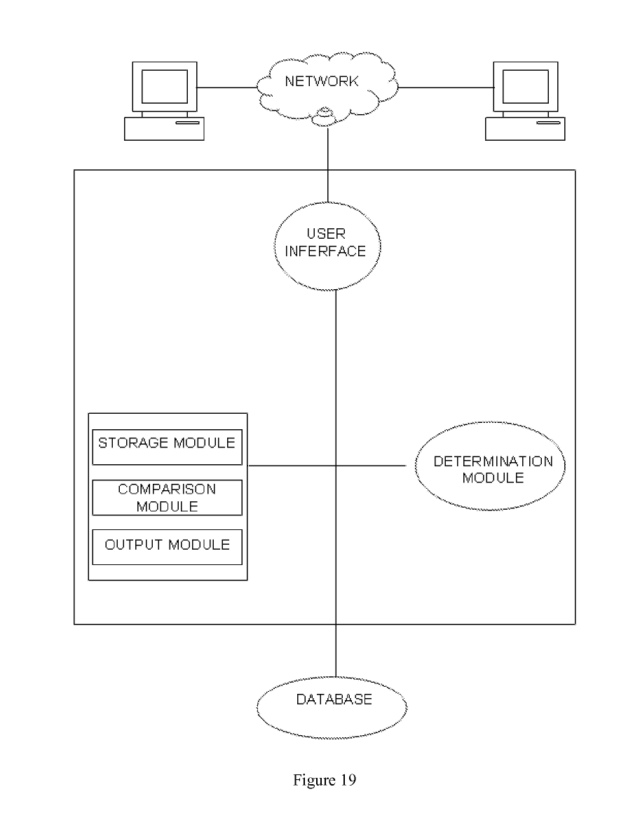

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers (FIG. 19).

The computing and/or comparison module provides a computer readable comparison result that can be processed in computer readable form by predefined criteria, or criteria defined by a user, to provide a content-based in part on the comparison result that may be stored and output as requested by a user using an output module, e.g., a display module.

In some embodiments, the content displayed on the display module can be the relative levels of pSer157-VASP, pSer239-VASP, or total VASP, in the epithelial tissue sample obtained from a subject, as described herein, as compared to a reference level. In certain embodiments, the content displayed on the display module can indicate whether pSer157-VASP, pSer239-VASP, and/or total VASP were found to have a statistically significant lower level of expression in the epithelial tissue sample obtained from a subject as compared to a reference level. In certain embodiments, the content displayed on the display module can indicate the degree to which pSer157-VASP, pSer239-VASP, or total VASP, were found to have a statistically significant lower level of expression in the epithelial tissue sample obtained from a subject as compared to a reference level. In certain embodiments, the content displayed on the display module can indicate whether the subject has an increased risk of having epithelial cancer. In certain embodiments, the content displayed on the display module can indicate whether the subject has an increased risk of epithelial cancer tumor initiation. In certain embodiments, the content displayed on the display module can indicate whether the subject has an increased risk of epithelial cancer metastasis. In certain embodiments, the content displayed on the display module can indicate whether the subject is in need of a treatment for epithelial cancer. In some embodiments, the content displayed on the display module can be a numerical value indicating one of these risk or probabilities. In such embodiments, the probability can be expressed in percentages or a fraction. For example, higher percentage or a fraction closer to 1 indicates a higher likelihood of a subject having epithelial cancer. In some embodiments, the content displayed on the display module can be single word or phrases to qualitatively indicate a risk or probability. For example, a word “unlikely” can be used to indicate a lower risk for having epithelial cancer, while “likely” can be used to indicate a high risk for having epithelial cancer.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for assessing the state of the epithelial tissue of a subject by measuring the level of pSer157-VASP, pSer239-VASP, or total VASP, described herein, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Kits for Diagnosis and Prognosis

In some embodiments, the invention provides a kit designed for facilitating the diagnosis of an epithelial tumor in a human comprising: (a) at least one solid support; (b) at least one antibody-based moiety specific for pSer157-VASP, pSer239-VASP or total VASP and (c) instruction for use and interpretation of the kit. Such a kit allows the determination of the levels of pSer157-VASP, pSer239-VASP and/or total VASP in a tumor, epithelial tissue or a biological sample obtained from a patient using an antibody-based method (e.g. ELISA).

In some embodiments, the kit further comprises (a) at least one solid support; (b) at least one antibody-based moiety specific for pSer157-VASP, pSer239-VASP or total VASP; (c) at least one labeled antibody-based moiety specific for the protein of (b); and (d) instruction for use and interpretation of the kit.

In some embodiments, for the kits described herein, the solid support is contacted with a biological sample from a subject. The biological sample can be as described elsewhere herein.

In some embodiments, the kits described herein further comprise standards of known amounts of pSer157-VASP, pSer239-VASP, and/or total VASP. In other embodiments, the kits described herein further comprise reference values of the levels of pSer157-VASP, pSer239-VASP, and/or total VASP. These reference values allow the determination of whether the levels of pSer157-VASP, pSer239-VASP, and/or total VASP in the tumor of the subject, epithelial tissue of the subject, or the biological sample obtained from the subject are reduced by certain percentages as compared to the reference levels; wherein reductions of the magnitudes listed in Table 1 indicate a diagnosis or prognosis as described therein and described further herein elsewhere. Reference values can be provided as numerical values, or as standards of known amounts of pSer157-VASP, pSer239-VASP, and/or total VASP.

In some embodiments, the reference values are as described elsewhere herein.

In one embodiment, for the kits described herein, at least one of the antibody-based moieties is immobilized on at least one solid support. In some embodiments of the kits described herein, at least one of the antibody-based moieties is immobilized on a first solid support and at least another of the antibody-based moieties is immobilized on a second solid support. In some embodiments of the kits described herein, each of the antibody-based moieties is immobilized on the same solid support, such as test strips. Such a kit allows simultaneous testing of more than one biomarker at one time. In one embodiment of the kits described herein, at least one of the antibody-based moieties is disposed upon but not immobilized on at least one solid support. Such the antibody-based moiety is mobile on the solid support, e.g. when in an aqueous solution.

Any solid support can be used, including but not limited to, nitrocellulose membrane, nylon membrane, solid organic polymers, such as polystyrene, solid beads or laminated dipsticks such as described in U.S. Pat. No. 5,550,375. The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

Examples of kits include but are not limited to ELISA assay kits, and kits comprising test strips and dipsticks. In an ELISA kit, an excess amount of antibody-based moieties specific for a particular antigen, in this case, pSer157-VASP, pSer239-VASP, and/or total VASP, is immobilized on a solid support. A sample containing an unknown amount of pSer157-VASP, pSer239-VASP, and/or total VASP is added to the immobilized antibody-based moiety, resulting in the formation of a complex consisting of pSer157-VASP, pSer239-VASP, and/or total VASP and the antibody-based moiety. The complex is detected by a labeled second antibody-based moiety that is also specific for pSer157-VASP, pSer239-VASP, and/or total VASP. The amount of label detected is a measure of the amount of biomarker present in the sample.

In some embodiments of the kits described herein, the kit comprises a test strip or a dipstick. In some embodiments of the kits described herein, the kit comprises a dot blot.

In some embodiments, when the solid support of the kits described herein is contacted with a sample containing pSer157-VASP, pSer239-VASP, and/or total VASP, an antibody-based moiety: pSer157-VASP, pSer239-VASP, and total VASP complex is formed. In some embodiments, the antibody-based moiety: pSer157-VASP, pSer239-VASP, and total VASP complexes are detected.

In some embodiments of the kits described herein, the labeled antibody-based moieties are detectably labeled. In some embodiments, the detectable label is selected from a group consisting of enzyme, fluorescent, biotin, gold, latex, hapten and radioisotope labeling. Detectable haptens include but are not limited to biotin, fluorescein, digoxigenin, dinitrophenyl (DNP). Other labels include but are not limited to colloidal gold and latex beads. The latex beads can also be colored. Methods of labeling antibodies, antibody-based moiety, or proteins are known in the art, for example, as described in “Colloidal Gold. Principles. Methods and Applications”, Hayat M A (ed) (1989-91). Vols 1-3, Academic press, London; in “Techniques in Immunocytochemistry”, Bullock G R and Petrusz P (eds) (1982-90) Vols 1, 2, 3, and 4, Academic Press, London; in “Principles of Biological Microtechnique”, Baker J R (1970), Methuen, London; Lillie RD (1965), Histopathologic Technique and practical Histochemistry, 3rd ed. McGraw Hill, New York; Berryman M A, et al (1992), J. Histochem Cytochem 40, 6, 845-857, all of which are incorporated hereby reference in their entirety.

In colloidal gold labeling technique, the unique red color of the accumulated gold label, when observed by lateral or transverse flow along a membrane on which an antigen is captured by an immobilized antibody, or by observation of the red color intensity in solution, provides an extremely sensitive method for detecting sub nanogram quantities of proteins in solution. A colloidal gold conjugate consists of a suspension of gold particles coated with a selected protein or macromolecule (such as an antibody or antibody-based moiety). The gold particles may be manufactured to any chosen size from 1 nm to 250 nm. This gold probe detection system, when incubated with a specific target, such as in a tissue section, will reveal the target through the visibility of the gold particles themselves. For detection by eye, gold particles will also reveal immobilized antigen on a solid phase such as a blotting membrane through the accumulated red color of the gold sol. Silver enhancement of this gold precipitate also gives further sensitivity of detection. Suppliers of colloidal gold reagents for labeling are available from SPI-MARKT™. Polystyrene latex Bead size 200 nm colored latex bead coated with antibody SIGMA ALDRICH®, Molecular Probes, Bangs Laboratory Inc., and AGILENT® Technologies.

In other embodiments of the kits described herein, at least one of the labeled antibodies comprises an enzyme-labeled antibody-based moiety. pSer157-VASP, pSer239-VASP, and/or total VASP that is bound and captured by the immobilized antibody-based moiety on the solid support (e.g. microtiter plate wells) is identified by adding a chromogenic substrate for the enzyme conjugated to the anti-antibody-based moiety and color production detected by an optical device such as an ELISA plate reader.

Other detection systems can also be used, for example, a biotin-streptavidin system. In this system, one of the antibodies (either the antibody-based moiety immunoreactive (i.e. specific for) with the biomarker of interest or the antibody-based moiety immunoreactive with that specific antibody) is biotinylated. The non-biotinylated antibody is incubated with wells coated with the biomarker antigen. Quantity of biotinylated antibody bound to the coated biomarker is determined using a streptavidin-peroxidase conjugate and a chromogenic substrate. Such streptavidin peroxidase detection kits are commercially available, e.g. from DAKO; Carpinteria, Calif.

Antibodies and antibody-based moiety can alternatively be labeled with any of a number of fluorescent compounds such as fluorescein isothiocyanate, europium, lucifer yellow, rhodamine B isothiocyanate (Wood, P. In: Principles and Practice of Immunoassay, Stockton Press, New York, pages 365-392 (1991)) for use in immunoassays. In conjunction with the known techniques for separation of antibody-antigen complexes, these fluorophores can be used to quantify the biomarker of interest. The same applies to chemiluminescent immunoassay in which case antibody or biomarker of interest can be labeled with isoluminol or acridinium esters (Krodel, E. et al., In: Bioluminescence and Chemiluminescence: Current Status. John Wiley and Sons Inc. New York, pp 107-110 (1991); Weeks, I. et al., Clin. Chem. 29:1480-1483 (1983)). Radioimmunoassay (Kashyap, M. L. et al., J. Clin. Invest, 60:171-180 (1977)) is another technique in which antibody can be used after labeling with a radioactive isotope such as 1251. Some of these immunoassays can be easily automated by the use of appropriate instruments such as the IMX™ (Abbott, Irving, Tex.) for a fluorescent immunoassay and Ciba Coming ACS 180™ (Ciba Corning, Medfield, Mass.) for a chemiluminescent immunoassay.

In some embodiments, the kits described herein further comprise instructions for using the kits and interpretation of results.

Unless otherwise stated, the present invention can be performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906): U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

### DEFINITIONS

For convenience, the meaning of certain terms and phrases used in the specification, examples, and appended claims, are provided below. If there is an apparent discrepancy between the usage of a term in the art and its definition provided herein, the definition provided within the specification shall prevail.

Definitions of common terms in cell biology and molecular biology can be found in “The Merck Manual of Diagnosis and Therapy”, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8); The ELISA guidebook (Methods in molecular biology 149) by Crowther J. R, (2000); Fundamentals of RIA and Other Ligand Assays by Jeffrey Travis, 1979, Scientific Newsletters; Immunology by Werner Luttmann, published by Elsevier, 2006. Definitions of common terms in molecular biology are also be found in Benjamin Lewin, Genes IX, published by Jones & Bartlett Publishing, 2007 (ISBN-13: 9780763740634); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values described herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data are provided in a number of different formats, and that these data, represent endpoints, starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

“Optional” or “optionally” means that the subsequently described event or circumstance may or may not occur and that the description includes instances where said event or circumstance occurs and instances where it does not.

The word “or” as used herein means any one member of a particular list and also includes any combination of members of that list.

The terms “decrease”, “reduced”, “reduction”, “decrease” or “inhibit” are all used herein generally to mean a decrease by a statistically significant amount when compared to an appropriate reference level. In one embodiment, the reduction is at least 10% as compared to a reference level, or at least about 20%, or at least about 30%, or at least about 40%, or at least about 50%, or at least about 60%, or at least about 70%, or at least about 80%, or at least about 90% or up to and including a reduction of 100% (e.g. absent level or non-detectable level as compared to a reference level).

As used herein, the term “administer” refers to the placement of a pharmaceutically acceptable composition into a subject by a method or route which results in at least partial localization of an effective amount of the composition to a desired site (e.g., to the epithelial neoplasm or tumor) such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebrospinal, and intrasternal injection and infusion. In preferred embodiments, the compositions are administered by intravenous infusion or injection.

As used herein in the context of expression, the terms “treat,” “treatment,” and the like, refer to an decrease in severity, indicators, symptoms, markers of epithelial neoplasm (e.g., tumor or cancer) as described herein. In the context of the present invention insofar as it relates to any of the conditions recited herein, the terms “treat,” “treatment,” and the like mean to relieve, alleviate, ameliorate, inhibit, slow down, reverse, or stop the progression, aggravation, deterioration, progression, anticipated progression or severity of at least one symptom or complication associated with epithelial neoplasm. In one embodiment, the onset of one or more symptoms of the neoplasm (e.g., epithelial tumor or cancer) are slowed by at least 5%, at least 10%, at least 20%, at least 30%, at least 40%, or at least 50%, or at least 60%, or at least 70%, or at least 80%, or at least 90%, or at least 99% that typically observed or expected in the absence of treatment. In one embodiment, the onset of one or more symptoms is completely prevented. In one embodiment, one or more of the symptoms of the neoplasm (e.g., epithelial tumor or cancer) are alleviated by at least 5%, at least 10%, at least 20%, at least 30%, at least 40%, or at least 50%, or at least 60%, or at least 70%, or at least 80%, or at least 90%, or at least 99% that experienced in the absence of treatment.

As used herein, the phrase “therapeutically effective amount”, refers to an amount that provides a therapeutic benefit in the treatment, prevention, or management of the condition (e.g., epithelial tumor or invasive epithelial cancer). A therapeutically effective amount is sufficient to provide a statistically significant decrease or otherwise measurable or detectable alleviation of at least one symptom of the condition, such as neoplastic growth, invasiveness, or metastasis into another organ (e.g., lymph nodes). Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “pharmaceutical composition” refers to the active agent in combination with a pharmaceutically acceptable carrier of chemicals and compounds commonly used in the pharmaceutical industry. The term “pharmaceutically acceptable carrier” excludes tissue culture medium.

The phrase “pharmaceutically acceptable” is employed herein to refer to those compounds, materials, compositions, and/or dosage forms which are, within the scope of sound medical judgment, suitable for use in contact with the tissues of human beings and animals to which the composition will contact, given the chose route of administration, without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one location in the body of a subject (e.g., the point of administration) to a targeted location or portion of the body (e.g., the position of the epithelial neoplasm, tumor, or cancer). A carrier is also “acceptable” in the sense of being compatible with the other ingredients of the formulation, for example the carrier does not decrease the impact of the agent on the treatment. An appropriate carrier is chosen with consideration for the route of administration.

The term “agent” refers to a composition that is utilized to produce the desired effect in a cell or a subject. The agent may normally be absent or may be typically present in the target cell, tissue or subject. Agents that can be used in the methods described herein can be chemicals; small molecules; nucleic acid sequences; nucleic acid analogues; proteins; peptides; aptamers; antibodies; or functional fragments thereof, and combinations thereof. A nucleic acid sequence can be RNA or DNA, and can be single or double stranded, and can be selected from a group comprising: nucleic acid encoding a protein of interest; oligonucleotides; and nucleic acid analogues; for example peptide-nucleic acid (PNA), pseudo-complementary PNA (pc-PNA), locked nucleic acid (LNA), etc. Such nucleic acid sequences include, but are not limited to nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, the term “antibody” refers to immunoglobulin molecules and immunologically active portions of immunoglobulin molecules, i.e., molecules that contain an antigen binding site that immunospecifically bind an antigen. The terms also refers to antibodies comprised of two immunoglobulin heavy chains and two immunoglobulin light chains as well as a variety of forms besides antibodies; including, for example, Fv, Fab, and F(ab)′2 as well as bifunctional hybrid antibodies (e.g., Lanzavecchia et al., Eur. J. Immunol. 17, 105 (1987)) and single chains (e.g., Huston et al., Proc. Natl. Acad. Sci. U.S.A., 85, 5879-5883 (1988) and Bird et al., Science 242, 423-426 (1988), which are incorporated herein by reference). (See, generally, Hood et al., Immunology, Benjamin, N.Y., 2ND ed. (1984), Harlow and Lane, Antibodies. A Laboratory Manual, Cold Spring Harbor Laboratory (1988) and Hunkapiller and Hood, Nature, 323, 15-16 (1986), which are incorporated herein by reference).

As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” and “RNA interfering” with respect to an agent of the invention, are used interchangeably herein.

As used herein a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

As used herein, “gene silencing” or “gene silenced” in reference to an activity of an exogenously added RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one embodiment, the mRNA levels are decreased by at least about 70%, about 80%, about 90%, about 95%, about 99%, about 100%, by the exogenously added RNAi molecule.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. In one aspect, the template nucleic acid is DNA. In another aspect, the template is RNA. Suitable nucleic acid molecules are DNA, including genomic DNA, ribosomal DNA and cDNA, Other suitable nucleic acid molecules are RNA, including mRNA. The nucleic acid molecule can be naturally occurring, as in genomic DNA, or it may be synthetic, i.e., prepared based up human action, or may be a combination of the two. The nucleic acid molecule can also have certain modification such as 2′-deoxy, 2′-deoxy-2′-fluoro, 2′-O-methyl, 2′-O-methoxyethyl (2-O-MOE), 2′-O-aminopropyl (2-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-0-N-methylacetamido (2-O-NMA), cholesterol addition, and phosphorothioate backbone as described in US Patent Application 20070213292; and certain ribonucleoside that are is linked between the 2′-oxygen and the 4′-carbon atoms with a methylene unit as described in U.S. Pat. No. 6,268,490, wherein both patent and patent application are incorporated hereby reference in their entirety.

The term “vector”, as used herein, refers to a nucleic acid construct designed for delivery to a host cell or transfer between different host cells. As used herein, a vector can be viral or non-viral.

As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain an exogenous gene in place of non-essential viral genes. The vector and/or particle may be utilized for the purpose of transferring any nucleic acids into cells either in vitro or in vivo. Numerous forms of viral vectors are known in the art.

The term “replication incompetent” as used herein means the viral vector cannot further replicate and package its genomes. For example, when the cells of a subject are infected with replication incompetent recombinant adeno-associated virus (rAAV) virions, the heterologous (also known as transgene) gene is expressed in the patient's cells, but, the rAAV is replication defective (e.g., lacks accessory genes that encode essential proteins from packaging the virus) and viral particles cannot be formed in the patient's cells.

As used herein, a “subject” means a human or animal Usually the animal is a vertebrate (e.g., a mammal) such as a primate, rodent, domestic animal (e.g., pet or farm animal) or game animal Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. Mammals other than humans can be advantageously used as subjects that represent animal models of epithelial cancer. The terms, “patient”, “individual” and “subject” are used interchangeably herein.

A subject can be male or female. A subject can be one who has been previously diagnosed with or identified as suffering from or having an epithelial neoplasm (e.g., epithelial tumor or epithelial cancer) or one or more complications related to an epithelial neoplasm, and optionally, but need not have already undergone treatment for the epithelial neoplasm or one or more complications related to the epithelial neoplasm. A subject can also be one who is not suffering from epithelial cancer. A subject can also be one who has been diagnosed with or identified as suffering from epithelial cancer or one or more complications related to epithelial cancer, but who show improvements in known epithelial cancer risk factors as a result of receiving one or more treatments for epithelial cancer or one or more complications related to epithelial cancer. Alternatively, a subject can also be one who has not been previously diagnosed as having epithelial cancer or one or more complications related to epithelial cancer. For example, a subject can be one who exhibits one or more risk factors for epithelial cancer or one or more complications related to epithelial cancer, or a subject who does not exhibit epithelial cancer risk factors, or a subject who is asymptomatic for epithelial cancer or one or more complications related to epithelial cancer. A subject can also be one who is suffering from or at risk of developing epithelial cancer or one or more complications related to epithelial cancer. A subject can also be one who has been diagnosed with or identified as having one or more complications related to epithelial cancer, or alternatively, a subject can be one who has not been previously diagnosed with or identified as having one or more complications related to epithelial cancer.

As used herein, a tumor can be benign or malignant. Epithelial tumors are tumors that arise from epithelial cells. Such tumors can arise from a variety of tissue types, including, without limitation, colon tissue, rectal tissue, lung tissue, breast tissue, prostate tissue, liver tissue, pancreatic tissue, kidney tissue, ovarian tissue, gastric tissue, oral tissue, esophageal tissue, skin tissue, intestinal tissue, stomach tissue, bladder tissue, and cervical tissue.

Malignant tumors are typically referred to as cancer. As used herein, “epithelial cancer” refers to cancers that arise from epithelial cells. Such cancers include, without limitation, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamous cell and basal cell cancers, prostate cancer, renal cell carcinoma, and other known cancers that effect epithelial cells throughout the body.

As used herein, the term “transforming” or “transformation” refers to changing an object or a substance, e.g., biological sample, nucleic acid or protein, into another substance. The transformation can be physical, biological or chemical. Exemplary physical transformation includes, but not limited to, pre-treatment of a biological sample, e.g., from whole blood to blood serum by differential centrifugation. A biological/chemical transformation can involve at least one enzyme and/or a chemical reagent in a reaction. For example, an exogenous molecule can be attached to one or more molecules in a test sample by the binding of a ligand and/or an antibody or antibody fragment.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip.

The term “software” is used interchangeably herein with “program” and refers to prescribed rules to operate a computer. Examples of software include: software; code segments; instructions; computer programs; and programmed logic.

The term a “computer system” may refer to a system having a computer, where the computer comprises a computer-readable medium embodying software to operate the computer.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The terms “t-statistic” and “t-stat” are used herein interchangeably and refer to a calculated numerical value which is used in a statistical test to indicate whether two samples have the same mean. As calculated herein, it is a measure of emphysema-associated signal relative to the noise. A negative t-stat indicates a gene that is expressed at lower levels as emphysematous damage increases, while a positive t-stat indicates a gene that is expressed at higher levels as emphysematous damage increases.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%. The abbreviation, “e.g.” is derived from the Latin exempli gratia, and is used herein to indicate a non-limiting example. Thus, the abbreviation “e.g.” is synonymous with the term “for example.”

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The invention is further illustrated by the following examples, which should not be construed as further limiting.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In colorectal cancer, the antitumorigenic guanylyl cyclase C (GCC) signalome is defective reflecting ligand deprivation from downregulation of endogenous hormone expression. Although the proximal intracellular mediators of that signal transduction system, including cyclic guanosine monophosphate (cGMP) and cGMP-dependent protein kinase (PKG), are well characterized, the functional significance of its distal effectors remain vague. Dysregulation of ligand-dependent GCC signaling through vasodilator-stimulated phosphoprotein (VASP), an actin-binding protein implicated in membrane protrusion dynamics, drastically reduced cGMP-dependent VASP phosphorylation levels in colorectal tumors from patients. Restoration of cGMP-dependent VASP phosphorylation by GCC agonists suppressed the number and length of locomotory (filopodia) and invasive (invadopodia) actin-based organelles in human colon cancer cells. Membrane organelle disassembly reflected specific phosphorylation of VASP Ser239, the cGMP/PKG preferred site, and rapid VASP removal from tumor cell protrusions. Importantly, VASP Ser239 phosphorylation inhibited the proteolytic function of invadopodia, reflected by suppression of the cancer cell ability to digest DQ-collagen IV embedded in Matrigel. These results demonstrate a previously unrecognized role for VASP Ser239 phosphorylation, a single intracellular biochemical reaction, as an effective mechanism which opposes tumor cell shape promoting colon cancer invasion and metastasis. Reconstitution of physiological cGMP circuitry through VASP, in turn, represents an attractive targeted approach for patients with colorectal cancer.

Despite considerable advancements in elucidating pathogenomonic genetic and molecular alterations, the causal mechanisms underlying initiation and progression of colorectal cancer, the third most common and deadly neoplasm in the western world, remain unknown. In the paracrine hormone hypothesis of colon cancer,¹ tumorigenesis is characterized by a state of guanylyl cyclase C (GCC) ligand insufficiency, in which expression of endogenous hormones guanylin and uroguanylin, secreted by and activating GCC receptors on intestinal epithelial cells in an autocrine and paracrine fashion, is dramatically reduced following a yet unidentified event early during transformation.²⁻³ Ligand-induced GCC activation physiologically regulates intestinal fluid and electrolyte homeostasis through intracellular accumulation of the second messenger cyclic guanosine monophosphate (cGMP) and cGMP-dependent protein kinase (PKG) phosphorylation of ion channels.⁴ Activation of GCC also promotes the transition from the proliferative crypt phenotype to the differentiated colonocyte along intestinal mucosal surfaces by imposing cytostasis⁵ and oxidative metabolic reprogramming,⁶ effects presumably underlying GCC-mediated colorectal antitumorigenesis.⁷ Thus, the GCC ligandopenia associated with neoplastic transformation may promote colorectal carcinogenesis by producing a repressed GCC and cGMP pathway devoid of tumor suppressor activities. However, the exact consequences at the molecular level of the absence of GCC activation in colon cancer cells are unclear.

A poorly investigated target for the antitumorigenic GCC pathway is the vasodilator-stimulated phosphoprotein (VASP).⁸ A mammalian member of the highly conserved Ena/VASP family proteins, VASP acts as a molecular scaffold to bring together growing actin filaments with regulatory proteins at the cell leading edge.⁹ VASP controls cell spreading and migration by regulating the formation and stability of protrusive membrane structures driven by actin polymerization, including lamellipodia and filopodia, and the integrity of cell-matrix interactions at adhesion complexes.⁹ In that context, VASP exerts a pivotal role in filopodial dynamics, reflected by its localization at the filopodial tip complex where it promotes filopodia formation and elongation by recruiting the initial nanomachinery and imposing anti-capping pressure on actin filament barbed ends.¹⁰ Three critical domains mediates VASP/actin interactions:⁹ 1) the Ena/VASP homology 1 (EVH1) domain at the N-terminus, which binds to proteins with poly-proline II helix, including vinculin and zyxin; 2) the central prolin-rich region, which binds to proteins containing SH3 and WW domains and the globular (G)-actin binding profilin, and 3) the EVH2 domain at the C-terminus, which binds to both G- and filamentous (F)-actin and mediates VASP oligomerization, thereby promoting F-actin bundling and stabilization. Importantly, VASP harbors 3 phosphorylation sites (Ser157, Ser239 and Thr278) which are targeted with different affinities by PKG, cAMP-dependent protein kinase and AMP-activated protein kinase, and modulate its localization and function.¹¹⁻¹³ Phosphorylation of VASP Ser239, adjacent to the G-actin binding site in the EVH2 domain, is selectively regulated by cGMP-dependent signaling through PKG,11 an event that disrupts VASP anti-capping and filament-bundling activities and inhibits membrane protrusion formation.^{9, 14}

While its engagement in migration and membrane protrusion dynamics in normal cells is well-established, a similar role for VASP Ser phosphorylation in cancer cells has not emerged. However, actin cytoskeletal remodeling at dynamic membrane regions determines oncogenic behaviors, from epithelial-to-mesenchymal transition to tissue invasion and metastasis.¹⁵⁻¹⁶ In principle, PKG-mediated VASP phosphorylation could represent one of the biochemical reactions of the antitumorigenic GCC/cGMP signalome.¹ As described herein, human colon tumors exhibited reduced GCC ligand expression associated with depletion of cGMP-dependent VASP phosphorylation, indicating interruption of downstream signaling by the dysregulated GCC pathway. Restoration of cGMP-mediated phosphorylation of VASP Ser239 by ligand-induced GCC signaling suppressed the invasive cancer cell phenotype, with disassembly of protrusive membrane organelles (filopodia. invadopodia) and inability to promote DQ-collagen IV degradation. Together, these observations reveal a previously unrecognized suppressor of invasive membrane protrusions and therapeutic target, VASP Ser239 phosphorylation, for colorectal cancer.

### Dephosphorylation of Ser157 and Ser239 of VASP is a Biomarker for Epithelial Cancer

VASP is an Intracellular Effector of the GCC Pathway in Colon Cancer Cells.

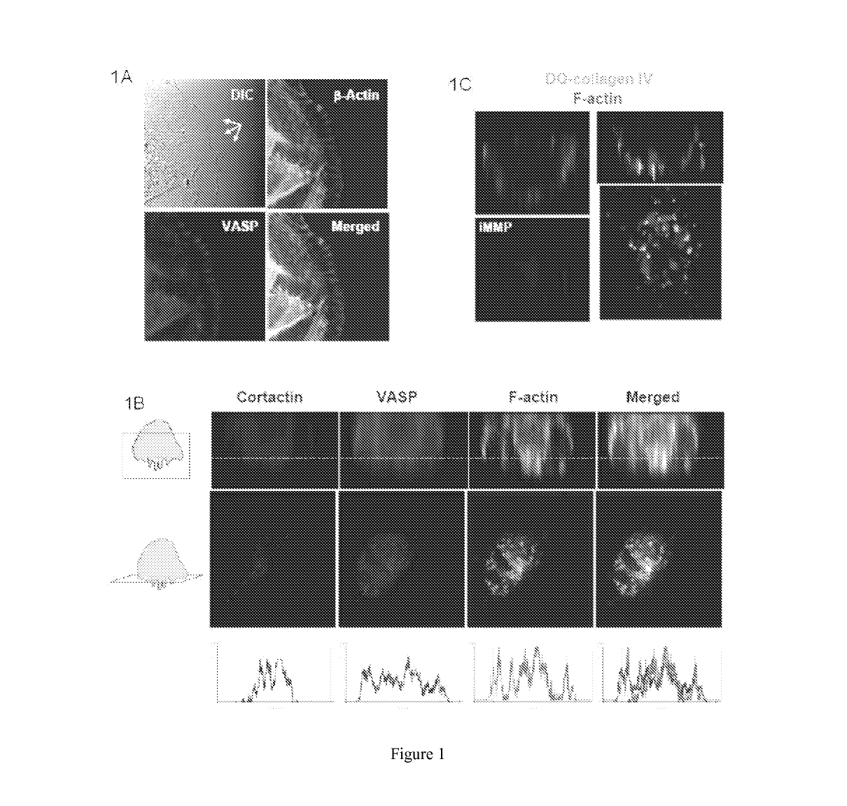

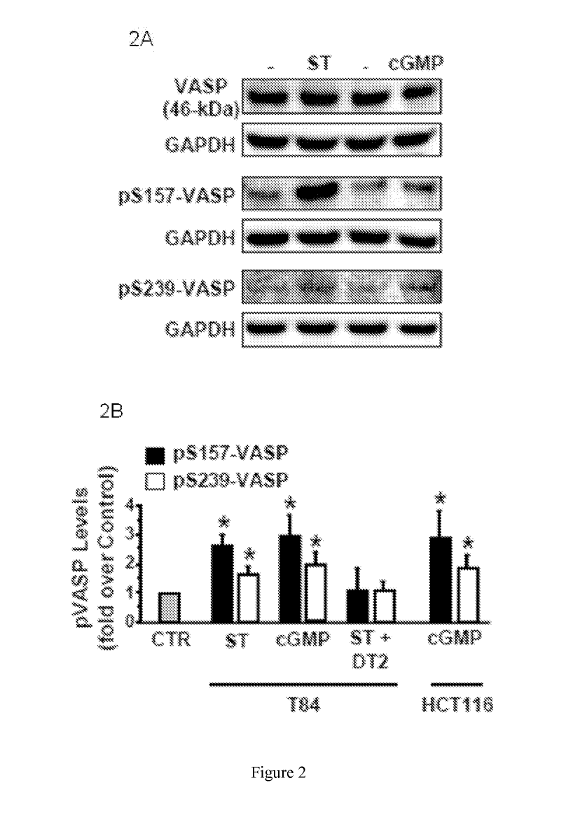

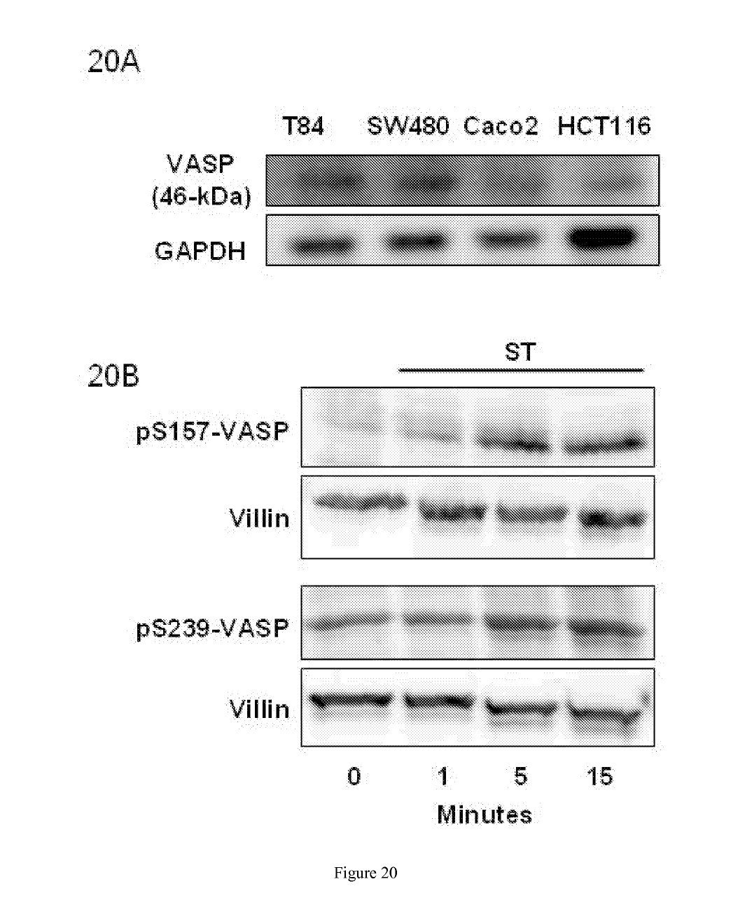

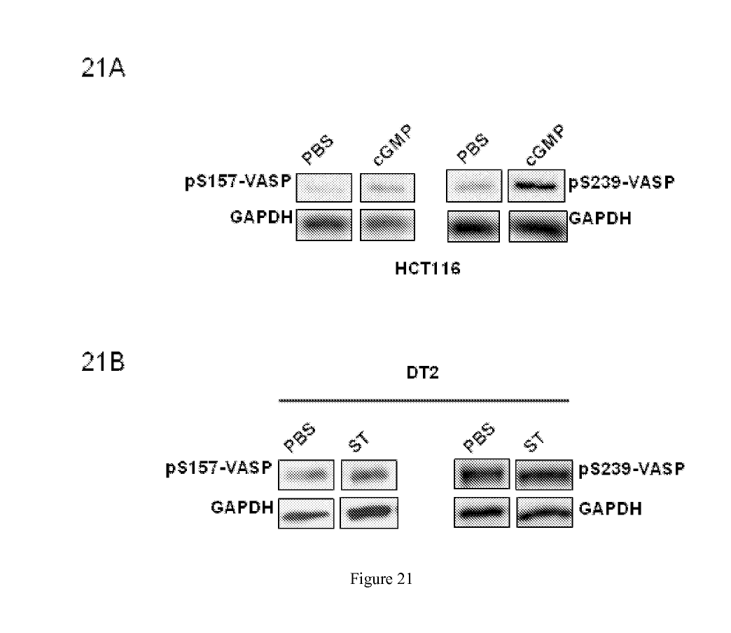

VASP is expressed in colon cancer cells (46-kDa band; FIG. 20A) and distributes to dynamic membrane domains, including cell leading edges (FIG. 1A) and invadopodia (FIG. 1B), where it colocalizes with cortactin and actin (FIG. 1B, right panels). Invadopodia identity was confirmed by assessing their ability to focally degrade DQ-collagen IV (FIG. 1C), a fluorogenic analog of the basement membrane component collagen IV. In addition, VASP is an intracellular target of ligand-dependent GCC signaling in human colon cancer cells,⁸ and the potent GCC agonist ST4 induced rapid phosphorylation of VASP at both Ser157 and Ser239 (FIG. 20B). ST effects on VASP were mimicked by the membrane-permeant cGMP analog 8-br-cGMP (FIGS. 2A-2B; FIG. 21A) and blocked by the selective PKG peptide inhibitor DT217 (FIG. 2B; FIG. 21B), confirming that ST induces VASP Ser phosphorylation by activating the GCC/cGMP/PKG pathway in human colon cancer cells.

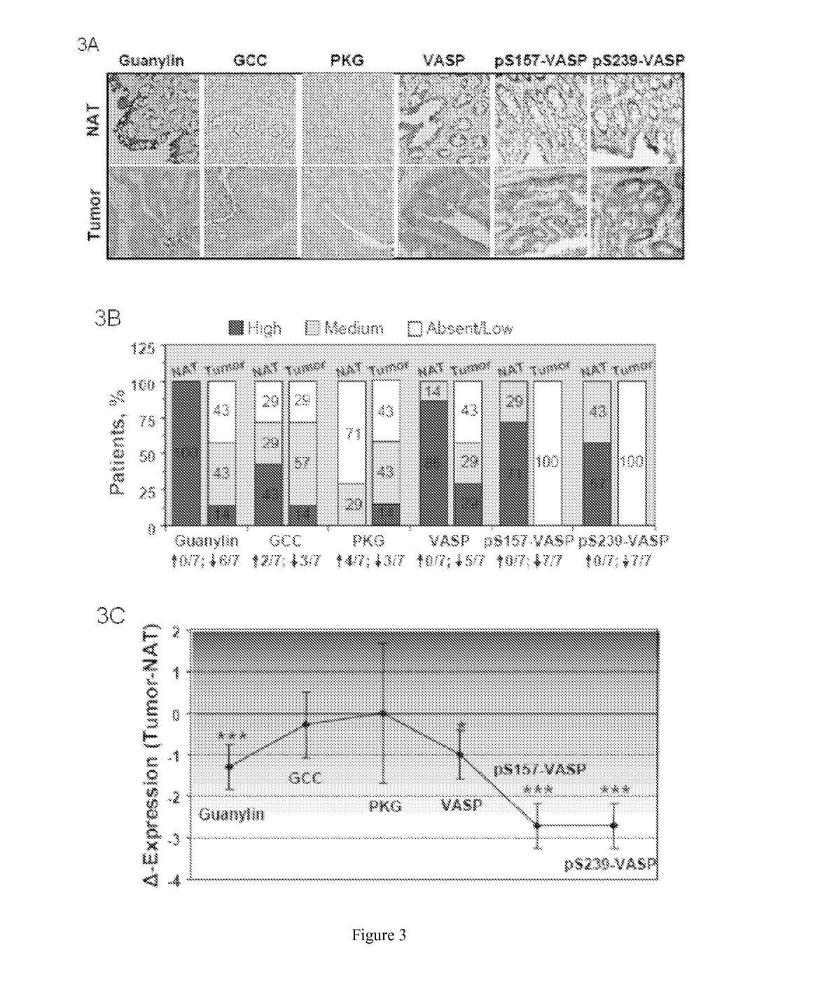

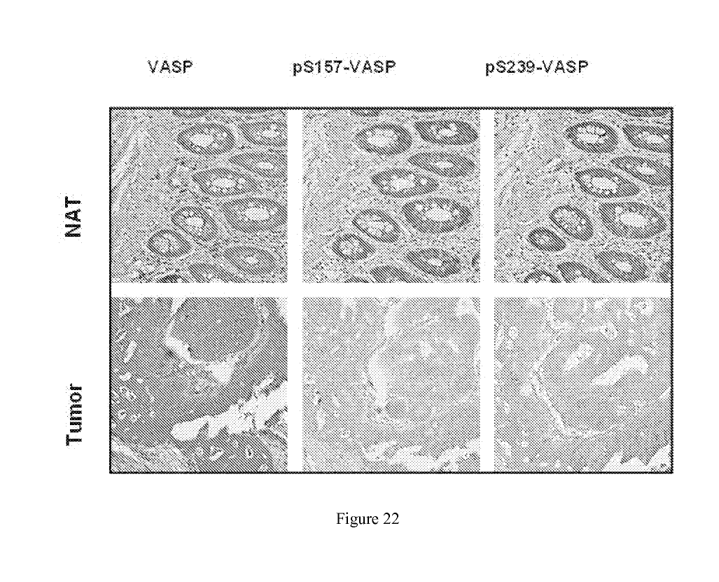

GCC signaling through VASP is dysregulated in colon cancer. In the human colon, the GCC pathway is constitutively activated by the paracrine hormone guanylin, which induces cGMP- and PKG-dependent phosphorylation of specific substrates.^{1, 4} Accordingly, normal colonic mucosa from patients exhibited epithelial cells with VASP Ser157 and Ser239 phosphorylation in the context of an intact guanylin-VASP signaling axis (FIG. 3A). However, following transformation and loss of endogenous ligand expression²⁻³ this GCC pathway becomes dysregulated (FIG. 3A), While GCC and PKG exhibited mixed trends with no significant changes, expression of guanylin and all the VASP species were significantly reduced in colorectal adenocarcinomas compared to their respective normal counterparts (FIGS. 3A-3C). Of significance, VASP phosphorylation at Ser157 and Ser239 were the most compromised signals of the GCC pathway, reflected by their downregulation in all tumors examined (FIG. 3B) and maximal loss of expression (FIG. 3C). Thus, loss of VASP Ser phosphorylation in colon cancer reflects interruption of effective induction and transmission of GCC-VASP signaling, beyond the mere changes in VASP expression (FIG. 22).

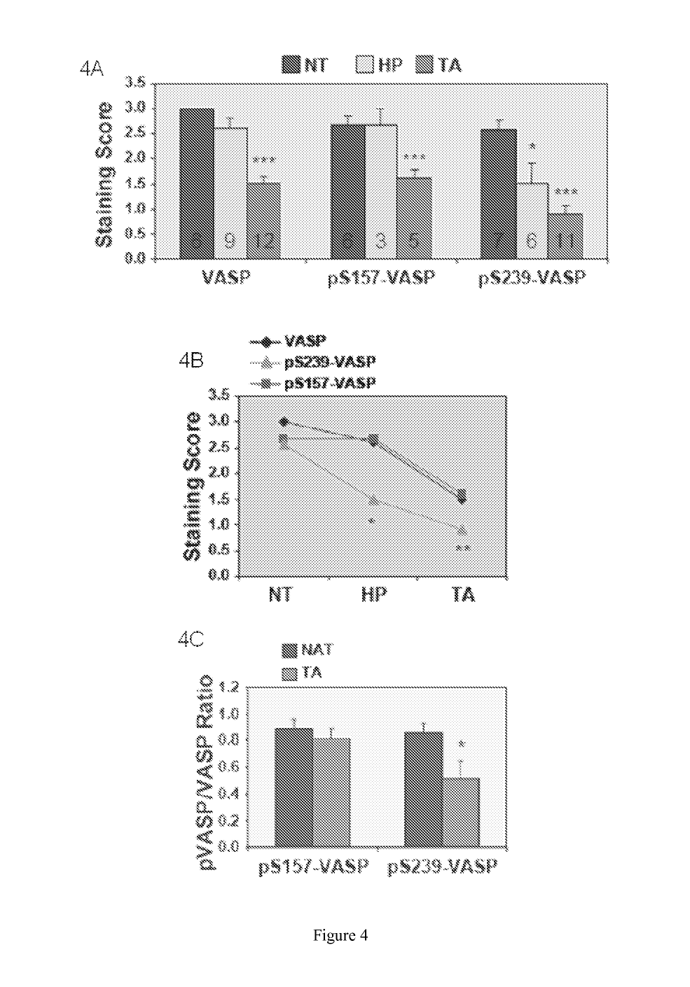

Downregulation of VASP, pSer157-VASP and pSer239-VASP predicts benign and pre-malignant neoplastic lesions in the colon and rectum. Paraffin-embedded tissue sections (5 μm) from patients with colorectal polyps, including benign (hyperplastic polyp, HP) and pre-cancerous (tubular adenoma, TA) lesions, were subjected to sequential steps of deparaffinization, rehydratation and antigen retrieval, followed by IHC for total VASP, pSer157-VASP and pSer239-VASP (FIGS. 4A-4C). Controls (normal tissues, NT) included normal colonic biopsies and patients' normal adjacent mucosa (NAT). The staining intensity of each signal (e.g., VASP; pSer157-VASP; pSer239-VASP) in epithelial cell compartments was semiquantitatively assessed by two blinded clinical pathologists with a 0 to 3 score (0, absent; 1, low; 2, moderate; 3, intense). Although they are present in all specimens examined, the levels of VASP and its Ser-phosphorylated species decrease in early colorectal transformation (FIGS. 4A-4C). Specifically, VASP, pSer157-VASP and pSer239-VASP were significantly reduced in pre-malignant polyps (tubular adenoma) compared to normal colonic mucosa (FIGS. 4A-4B). Importantly, pSer239-VASP (but not VASP or pSer157-VASP) levels already dropped at the earliest stage of tumor initiation, the benign hyperplastic polyp (FIGS. 4A-4B). Further, while they were equivalent in the normal mucosa, pSer239-VASP staining intensity was significantly lower than that of VASP or pSer157-VASP in colonic tumor lesions (FIGS. 4A-4B), suggesting that decreased pSer239-VASP in colorectal polyps reflects mutational events beyond the mere reduction of total VASP protein levels. Indeed, the pSer239-VASP/VASP ratio (but not pSer157-VASP/VASP ratio) is altered in colorectal tumorigenesis, as indicated by its significant reduction in tubular adenomas compared to respective normal adjacent tissues (FIG. 4C). Accordingly, the pSer239-VASP/pSer157-VASP ratio also decreases −45% along the normal mucosa (0.96)-hyperplastic polyp (0.56)-adenoma (0.57) sequence underlying colorectal tumorigenesis. Together, these data suggest that total VASP, pSer157-VASP and pSer239-VASP represent biomarkers of pre-malignant transformation. Without wishing to be bound by theory, reduction of pSer157-VASP levels in colonic polyps may reflect total VASP downregulation. In contrast, depletion of pSer239-VASP levels is a more sensitive alteration that occurs at the earliest events of tumorigenesis, is of maximal magnitude and is associated with additional mutational events, beyond total VASP changes. Hence, absolute levels of pSer239-VASP, and pSer239-VASP/VASP or pSer239-VASP/pSer157-VASP ratios are specific indices of tumor initiation and early malignant disease progression, which could be exploited as independent prognostic factors to diagnose both benign and pre-cancerous colorectal lesions in patients.

Loss of pSer239-VASP Indicates Colon Cancer Metastasis.

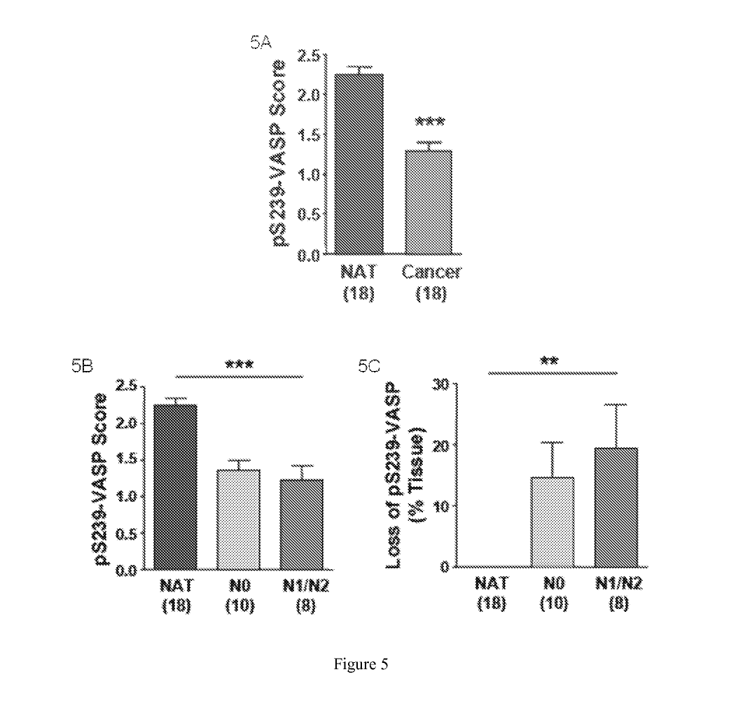

Because of its significance in colorectal tumorigenesis (see above), the role of pSer239-VASP as a biomarker of metastatic disease progression was evaluated. Tumors (Cancer) and normal adjacent tissues (NAT) from 18 patients with colorectal adenocarcinomas were subjected to IHC for pSer239-VASP (FIGS. 5A-5C). A specific pSer239-VASP staining score for each tissue slide was quantified by two blinded clinical pathologists as the product of the staining intensity level (0-3 scale: 0, absent; 1, low; 2, moderate; 3, intense) by the fraction of epithelial cells with that staining intensity. Levels of pSer239-VASP were significantly reduced in colorectal adenocarcinomas compared to their respective normal counterparts (FIG. 5A). Of relevance, expression of pSer239-VASP progressively decreases with increasing cancer invasion and lymph node involvement. Thus, pSer239-VASP levels exhibited a significant negative linear trend correlated with metastatic dissemination to regional lymph nodes, following one-way analysis of variance (ANOVA) of specific staining scores assigned to normal mucosa and primary colorectal adenocarcinomas with tumor negative (NO) or positive (N1/N2) lymph nodes (FIG. 5B). Conversely, quantification of percentages of epithelial cell compartments with complete loss of pSer239-VASP staining in the same clinical groups revealed that elimination of pSer239-VASP is positively correlated (by linear trend test following one-way ANOVA) with metastatic spreading of colorectal cancer cells (FIG. 5C). These results suggest that pSer239-VASP represents a biomarker of invasion and metastasis in colorectal cancer. Progressive depletion of pSer239-VASP levels in tumor epithelial cells of primary colorectal adenocarcinomas is associated with disease progression toward metastatic stages. Specifically, staining indices of pSer239-VASP or tissue percentages with pSer239-VASP loss are specific indicators of colorectal cancer metastasis, which could be exploited as independent prognostic factors to diagnose early tumor dissemination at mesenteric lymph nodes in patients.

### VASP is Therapeutic Target for Epithelial Cancer

VASP Ser239 Phosphorylation by GCC Activation Inhibits Cancer Cell Shape Mediating Migration and Invasion.

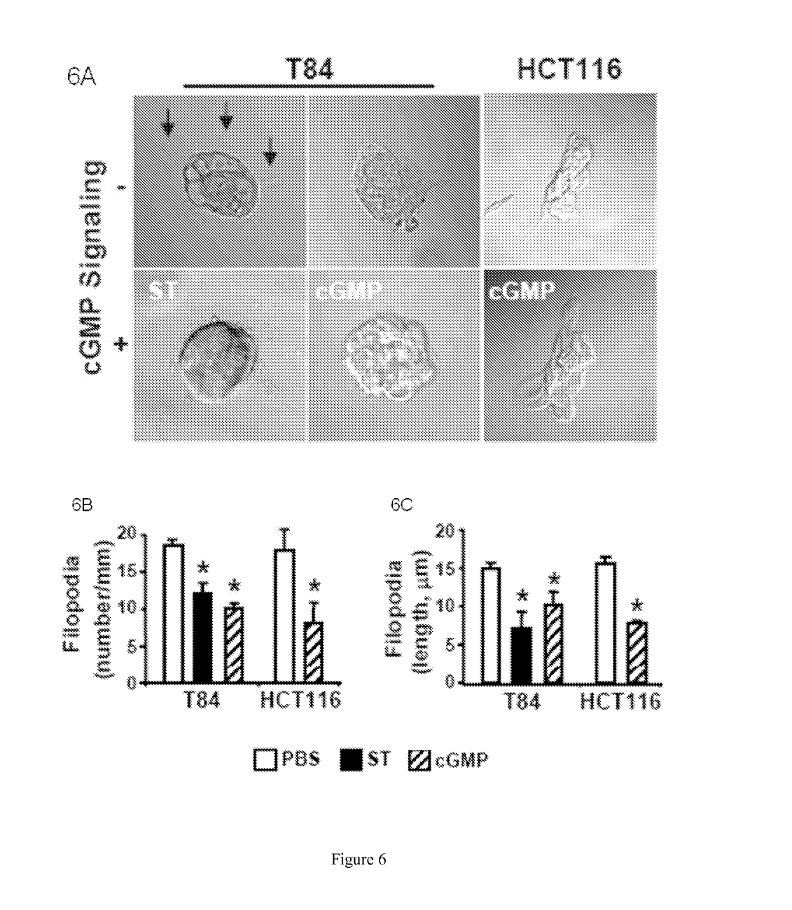

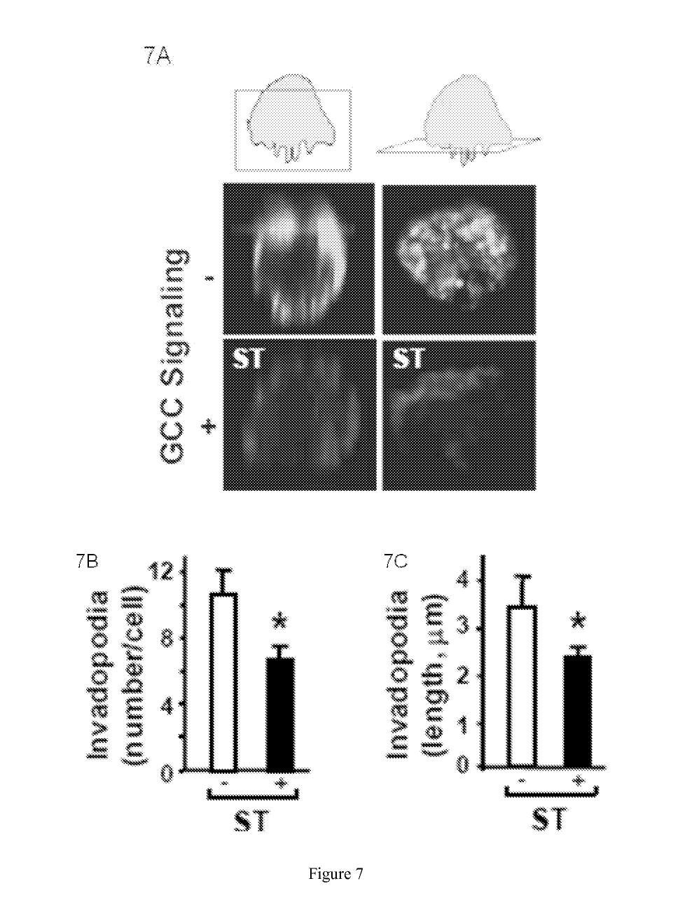

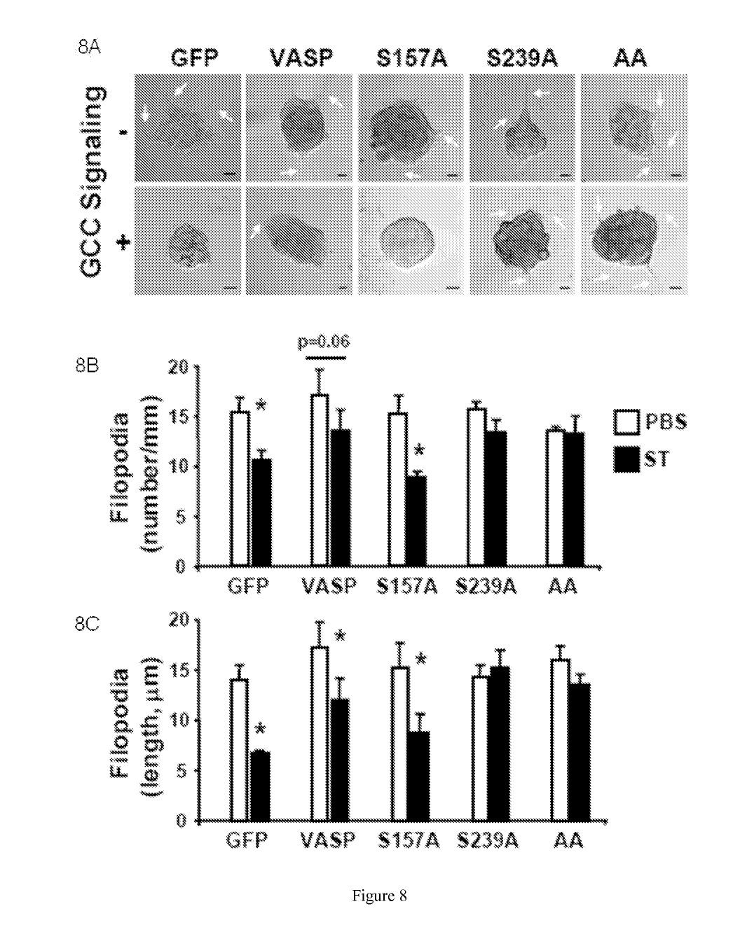

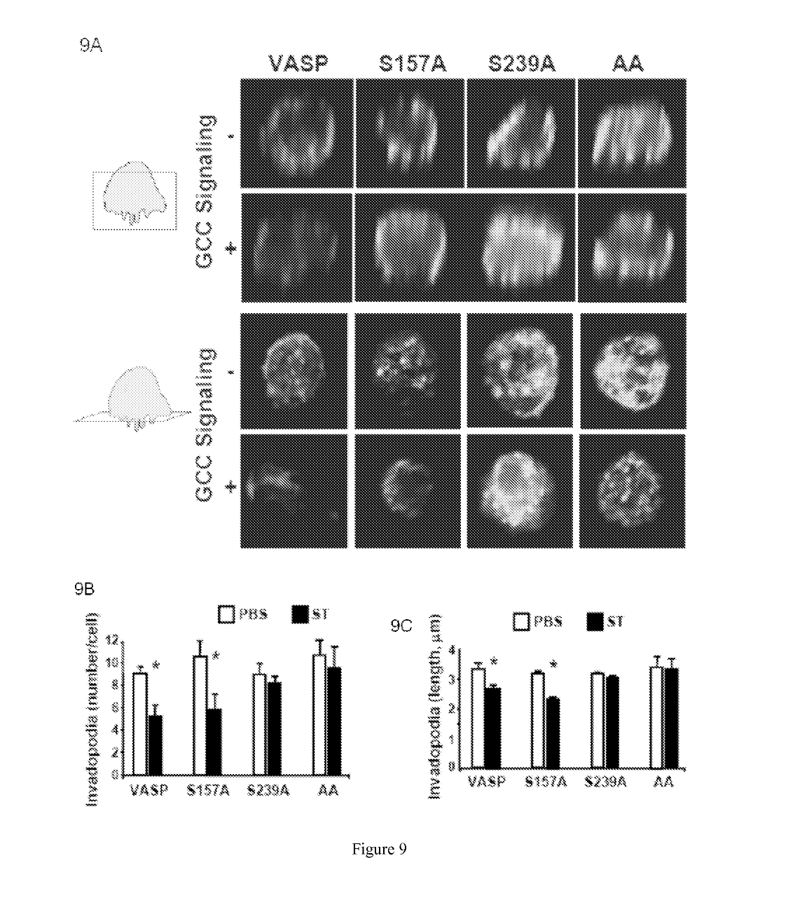

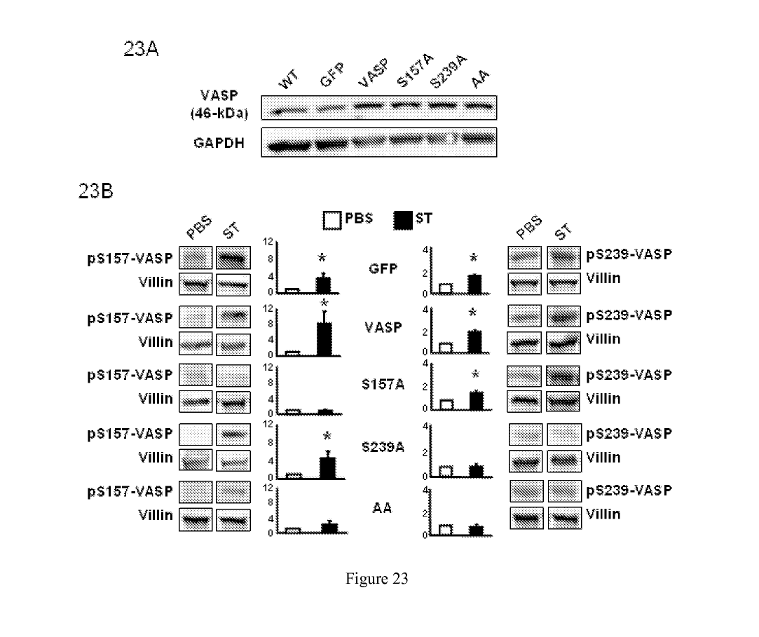

As VASP regulates membrane protrusion geometry¹⁸ and is present at cell leading edges and invadopodia (FIGS. 1A-1B), the functional consequences of ligand-dependent GCC signaling through VASP Ser phosphorylation on the membrane architecture of colon cancer cells were examined. GCC signaling through cGMP significantly reduced the number and length of migratory (filopodia; FIGS. 6A-6C) and invasive (invadopodia; FIGS. 7A-7C) actin-based protrusions. Filopodia identity was confirmed by specific immunostaining with the filopodia marker fascin (data not shown). Importantly, cGMP inhibitory effects were specifically mediated by induction of VASP Ser239 phosphorylation, as demonstrated by employing tumor cells stably expressing distinct VASP phosphomutants (FIG. 23A). Thus, cancer cells with VASP-S239A or VASP-AA, which contain point mutations at Ser239 and do not accept phosphorylation at that site (FIG. 23B), were resistant to GCC-mediated inhibition of filopodia (FIGS. 8A-8C) and invadopodia (FIGS. 9A-9C). In contrast, control cells expressing GFP or overexpressing VASP and tumor cells with the phosphomutant VASP-S157A, which harbors a point mutation at Ser157 only and does not accept phosphorylation at that site (FIG. 23B), were sensitive to suppression of filopodia (FIGS. 8A-8C) and invadopodia (FIGS. 9A-9C) upon ligand-dependent GCC signaling.

VASP Ser239 Phosphorylation Suppresses Invasive Membrane Protrusions by Inducing Intracellular VASP Redistribution.

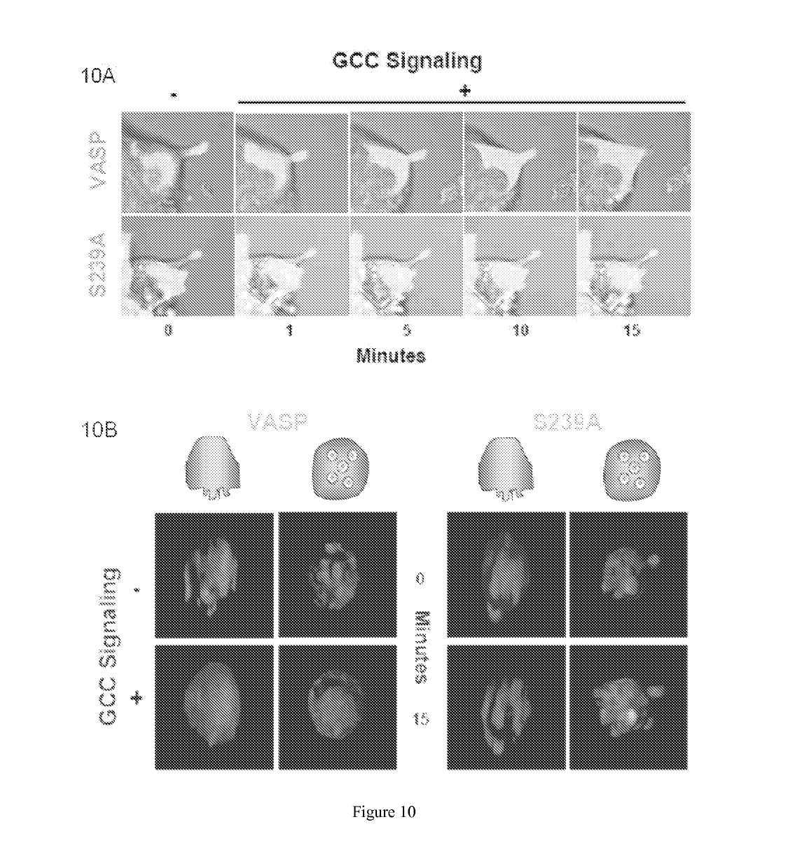

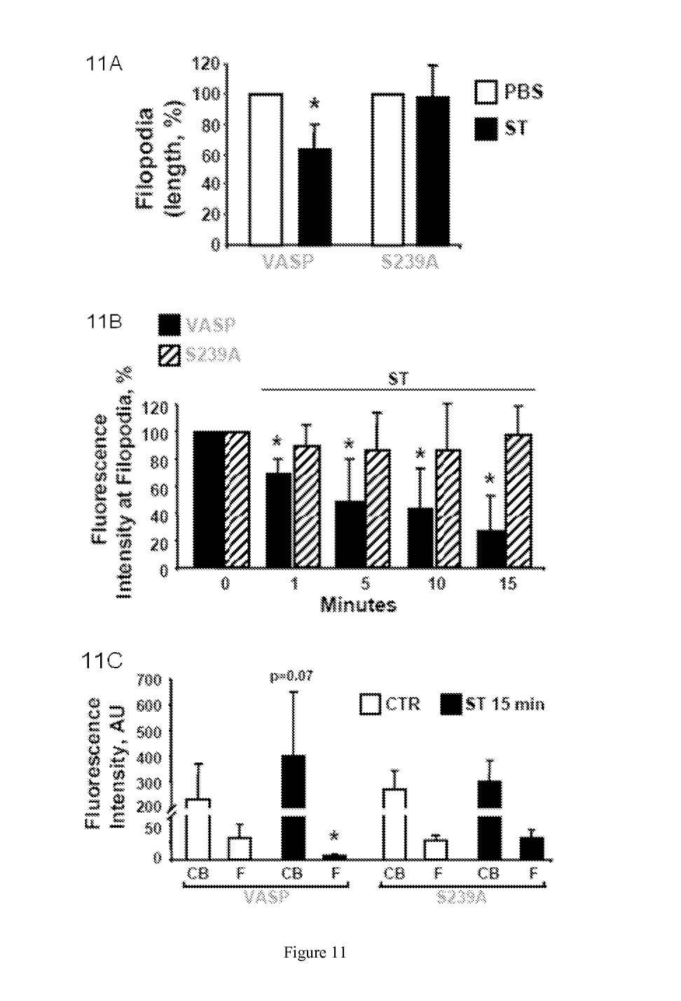

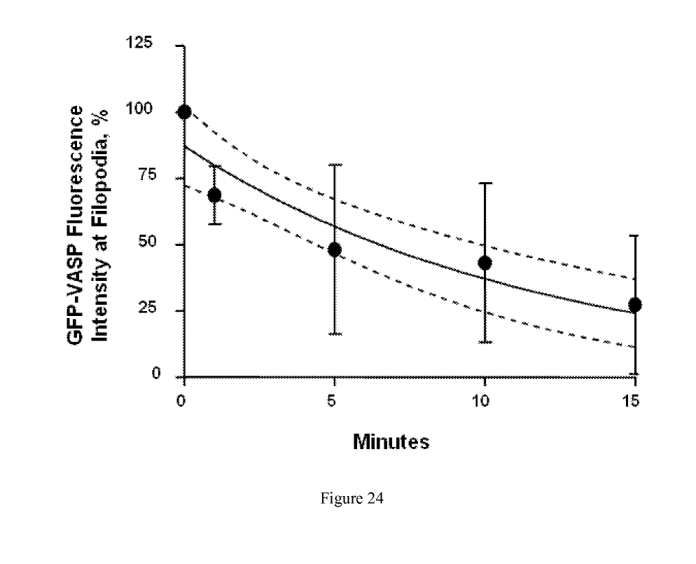

The molecular mechanism mediating inhibition of invasive cell shape by VASP Ser239 phosphorylation was investigated with live imaging microscopy in cells stably expressing GFP-VASP constructs (FIGS. 10A-10B, 11A-11C). Colon cancer cells with GFP-VASP, but not mutant cells expressing GFP-VASP-S239A, exhibited disassembly of filopodia (FIG. 10A) and invadopodia (FIG. 10B) upon ligand-dependent GCC activation. Protrusion retraction by GCC (FIG. 11A) reflected rapid removal of VASP from membrane projections (FIG. 11B), with a calculated half-life of 8.11 min (FIG. 24), and VASP redistribution to inner cell compartments away from membrane tips (FIG. 11C). However, the GFP-VASP-S239A mutant (resistant to Ser239 phosphorylation), was insensitive to GCC signaling (FIG. 11C), and retained its localization (FIG. 11B) and distribution (FIG. 11C) at locomotory membrane extensions, suggesting that VASP Ser239 phosphorylation represents a disassembly signal for invasive migratory organelles by uncoupling VASP from actin-based microregions at colon cancer cell membranes.

VASP Ser239 Phosphorylation Prevents DQ-Collagen IV Degradation by Colon Cancer Cells.

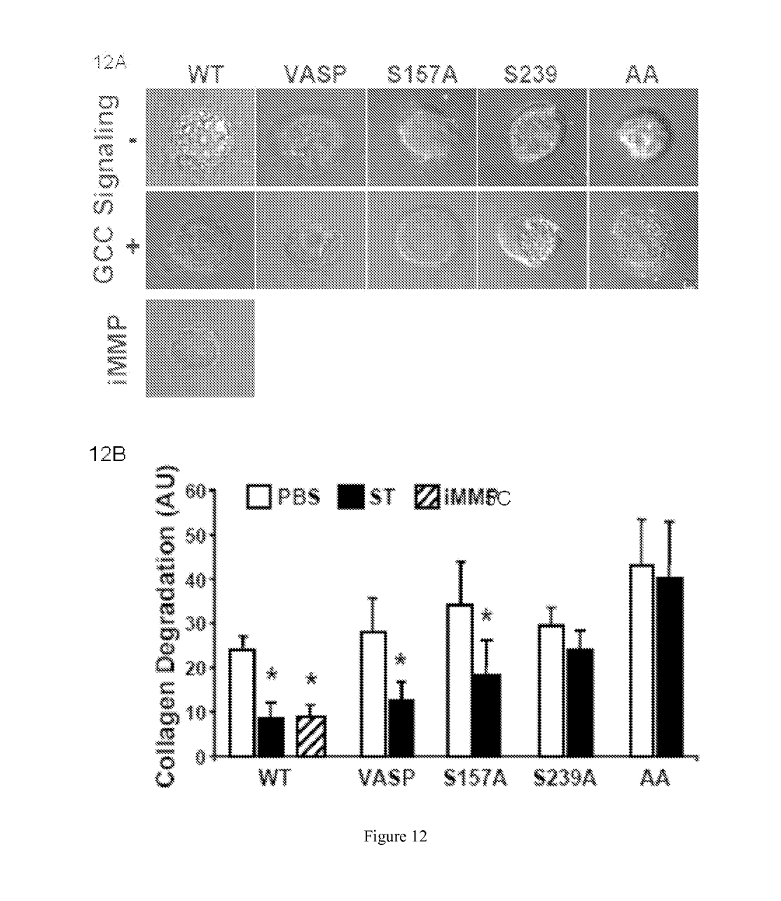

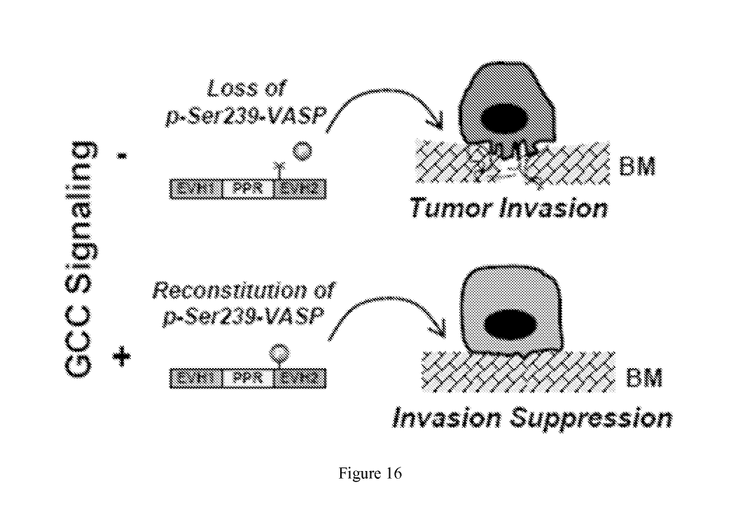

Metastatic cancer dissemination requires degradation and remodeling of the surrounding environment,¹⁹ a malignant attribute conferred to colon cancer cells by their invadopodia (FIG. 1C). Strikingly, activation of GCC signaling through VASP abolished the cancer ability to degrade DQ-collagen IV, with similar magnitude to that of a general inhibitor of MMPs (FIGS. 12A-12B), enzymes mediating tumor matrix degradation.²⁰ However, colon cancer cells with VASP-S239A or VASP-AA, but not VASP-S157A, did continue to proteolytically digest DQ-collagen IV following ligand-dependent GCC signaling (FIGS. 12A-12B), indicating that VASP Ser239 phosphorylation mediates arrest of invadopodia-mediated proteolysis by the GCC pathway. Thus, VASP Ser239 phosphorylation is a unique biochemical reaction that suppresses membrane protrusions promoting tumor invasion in colorectal cancer (FIG. 16).

pSer239-VASP Regulates Peritoneal Metastasis by Human Colon Cancer Cells.

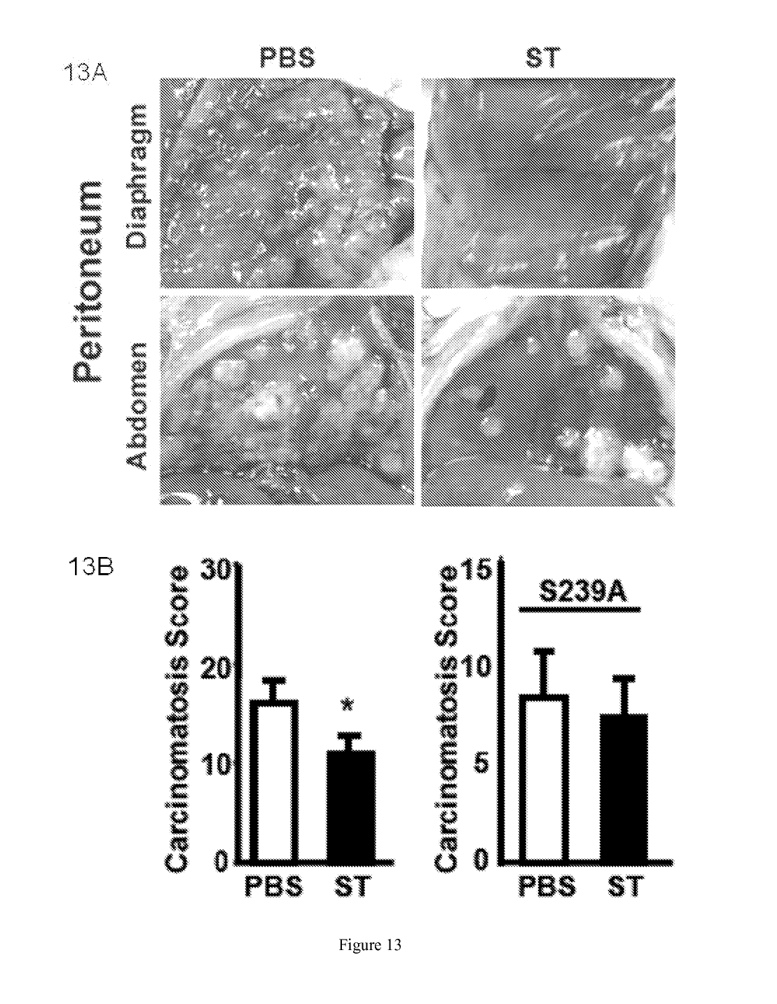

Preferred target tissues for colorectal cancer spreading and metastasis are the lung, liver and peritoneum. To study the impact of pSer239-VASP on colon cancer cell metastasis, the mouse model of peritoneal carcinomatosis was investigated. Since it is lost in colon cancer, pSer239-VASP levels in tumor cells were elevated with the bacterial enterotoxins ST, a guanylyl cyclase C (GCC) agonist inducing cGMP-dependent pSer239-VASP. Also, the VASP phosphomutant with serine-to-alanine substitution at the amino-acid position 239 (S239A) was stably expressed in T84 human colon cancer cells employing a mouse stem cell virus (MSCV) retroviral vector. Then, cancer cells were treated in vitro with PBS (control) or ST for 24 hours (h), injected into the peritoneal cavity of anesthetized male nude mice and resulting peritoneal tumors examined after 12 weeks (wk). Briefly, mice were sacrificed and peritoneal surfaces exposed to quantify the degree of carcinomatosis employing an index (0-30), reflecting the sum of tumor burden (on a 0 to 3 scale) in each of the 10 regions in which the peritoneum was subdivided (see legend to FIGS. 13A-13B). Treatment with ST inhibited the ability of human colon cancer cells to form peritoneal metastases (FIGS. 13A-B). However, tumor cells with the VASP mutant S239A, in which the cGMP-regulated Ser239 site is eliminated, were insensitive to ST effects (FIGS. 13A-13B), indicating that in colon cancer induction of pSer239-VASP prevents peritoneal carcinomatosis. These data demonstrate that pSer239-VASP is a metastasis suppressor for colon cancer, thereby supporting the notion that pSer239-VASP is a significant biomarker of the metastatic cell phenotype. Thus, loss of pSer239-VASP may represent a novel biological indicator of colorectal cancer metastasis.

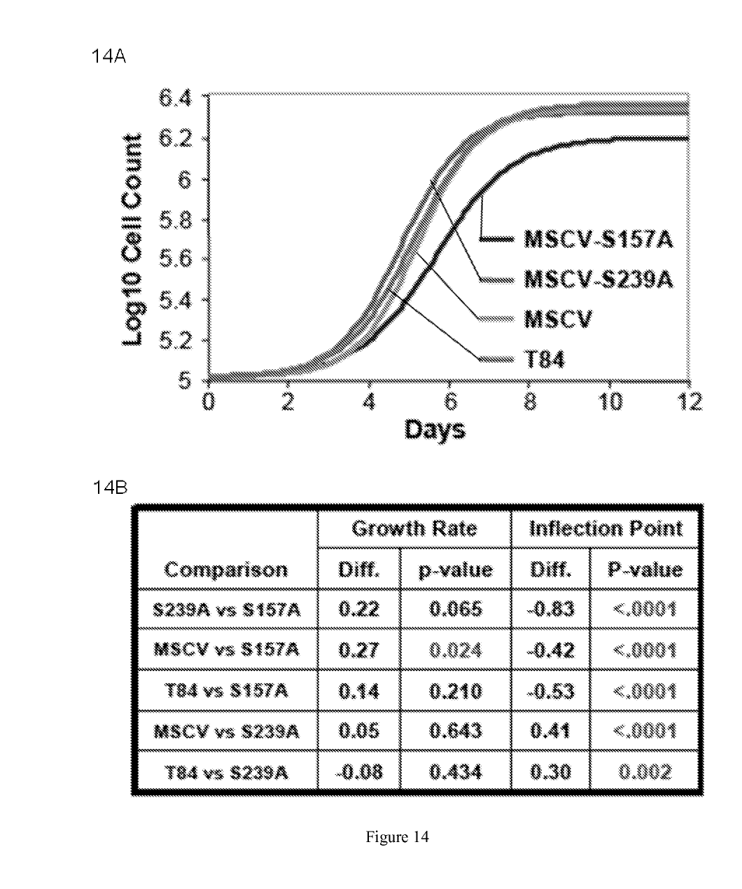

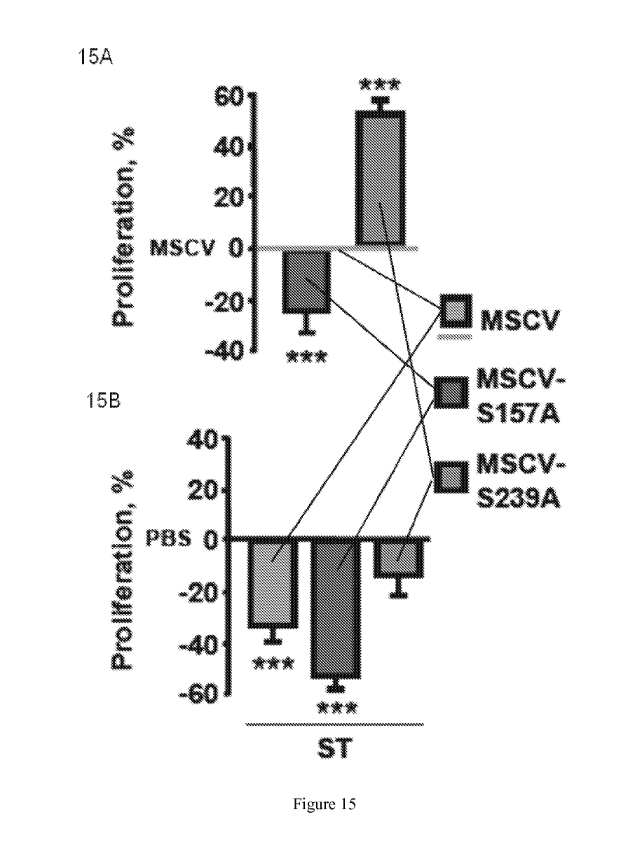

Genetic therapy with the VASP mutant S157A, alone or in combination with cGMP agonists, inhibits human colon cancer cell proliferation. The role of VASP Ser phosphorylation in colorectal tumorigenesis was further expanded by analyzing its impact on malignant cell proliferation. These studies employed VASP phosphomutants with alanine substitutions at Ser157 or Ser239, delivered to cancer cells with the MSCV vector. In this way, T84 human colon cancer cells stably expressed either the empty vector (MSCV) or the VASP phosphomutants MSCV-S157A or MSCV-S239A. Analysis of tumor growth curves demonstrates that the VASP mutant unable to be phosphorylated at Ser157, but not the mutant at Ser239, confers to colon cancer cells restricted growth kinetics, with significant lower growth rates and inflection points compared to controls (FIGS. 14A-14B). Interestingly, the VASP Ser mutants impose opposite effects on the ability of colon cancer cells to replicate their DNA. Thus, cells with the VASP S239A mutant exhibited increased DNA synthesis rates (FIG. 15A), indicating that pSer239-VASP is a tumor suppressor which orchestrates antiproliferative programs in colon cancer. Accordingly, induction of intracellular pSer239-VASP with ST suppresses cancer cell proliferation, an effect blocked by the VASP mutant S239A (FIG. 15B). In contrast, cells with the VASP S157A mutant exhibited decreased DNA synthesis rates (FIG. 15A), indicating that pSer157-VASP is a tumor promoter for colon cancer. Importantly, the combination of VASP S157A with ST (or cGMP analogs, data not shown) synergistically reduced colon cancer cell proliferation (FIG. 15B), offering an innovative therapeutic approach against colorectal tumorigenesis. These observations are the most relevant as they reveal a novel therapeutic paradigm for colorectal cancer which target VASP through inhibition of pSer157-VASP signaling. They indicate the VASP phosphomutant MSCV-S 157A is a novel genetic therapy to inhibit the growth and proliferation of colon cancer cells. In addition, they offer MSCV-S 157A plus cGMP agonists (e.g., ST, cGMP analogs) as combinatorial therapeutics with synergistic efficacy against colon cancer growth and progression. Thus, VASP S157A-based therapies represent innovative remedies to treat colorectal cancer in patients.

### Discussion

The antitumorigenic GCC signalome is deregulated during colorectal transformation reflecting loss of endogenous ligand expression.¹⁻³ Present findings reveal that this alteration affects a cGMP-dependent intracellular pathway comprising VASP as the distal effector, with subsequent silencing of an important biochemical reaction, VASP Ser239 phosphorylation, which opposes malignant cell shape. Interruption of GCC-VASP signaling, in turn, results in specific depletion of the VASP phosphospecies, which represent previously undescribed biomarkers for GCC deregulation and tumorigenesis in colon cancer.

Membrane protrusion extension principally reflects the balance of antagonistic forces at actin filament barbed ends imposing persistence (anti-capping proteins) or arrest (capping proteins) of actin polymerization.¹⁰ VASP is a critical anti-capping protein that initiates and maintains dynamic membrane regions by orchestrating a cytoskeletal assembly-line promoting simultaneous F-actin elongation and bundling.^{10, 22} In this way, Ena/VASP family members control migration and membrane protrusion formation underlying important physio(patho)logical processes, from wound-healing, platelet aggregation and neural development⁹ to inflammation, cancer spreading and metastasis.^{9, 23-24} Accordingly, expression of Ena/VASP proteins positively correlates with disease progression in patients with lung²⁵ or breast²⁶ cancer, suggesting their involvement in carcinogenesis and metastasis. The results described herein underscore the critical role of VASP in organizing cancer functions associated with the malignant membrane shape. In that context, although with unclear activities, VASP is a molecular component of invadopodia,²⁷ cancer cell projections mediating focal matrix proteolysis. Here, colon cancer cells required VASP for invadopodia stability and function, indicating that VASP maintains the structural integrity of these invasive protrusions. Indeed, intracellular VASP redistribution and removal from protrusive tips, upon GCC-dependent phosphorylation, destabilizes actin-driven membrane extensions (FIGS. 10A-10B, 11A-11C), resulting in protrusion retraction. Importantly, invadopodia represent attractive antimetastatic targets since they function as critical hubs of invasive cell behavior, spatially and temporally organizing the activities of actin nucleators and regulators (Arp2/3, N-WASP, cortactin), signaling enzymes (Src, Erk1/Erk2, FAK), adhesive receptors (integrins β1/β3, CD44), and proteases (MT1-MMP, MMP2, MMP9).²⁷⁻²⁸ Thus, the ability of cGMP-dependent VASP phosphorylation to dismantle invadopodia in colon cancer cells (FIGS. 9A-9C) offers an innovative antiinvadopodial strategy to be exploited against tumor metastasis in patients.

Tumor suppressors inhibiting metastatic cell dissemination are scarce. Their critical value resides in the potential to serve as biomarkers to improve clinical staging and targets to reduce cancer mortality. VASP Ser239 phosphorylation, a simple intracellular biochemical reaction, can represent a unique invasion suppressor with sophisticated regulatory dynamics, an inducible mechanism embedded into signal transduction networks shaping tumor cell metastasis. Further, inhibition of metastasis by VASP Ser239 phosphorylation can be a general strategy, as VASP is ubiquitously expressed across human tissues.⁹ Of significance, cGMP-dependent VASP Ser157 phosphorylation does not appear to have a causative relationship with the invasive phenotype of colon cancer cells (FIGS. 8A-8C, 9A-9C, 12A-12C). Since VASP Ser239 is selectively regulated by PKG-dependent phosphorylation,^{9, 14} while VASP Ser157 is a more promiscuous site phosphorylated by PKG, cAMP-dependent protein kinase, and protein kinase C-dependent and -independent mechanisms,^{11, 29} the results described herein suggest a unique role of the GCC/cGMP/PKG pathway in suppressing colorectal cancer invasion through VASP Ser239.

The results described herein also support the existence of a biological divergence between cGMP and cAMP signaling on Ena/VASP activities, an established paradigm in neurons where the cAMP-VASP axis mediates attractive guidance cues by increasing the number and length of filopodia, while the cGMP/PKG/VASP pathway promotes repulsive guidance cues.³⁰⁻³¹ However, the precise consequences of cyclic nucleotide signaling through VASP on the actin cytoskeleton appear to be cell and tissue specific, as demonstrated by identical cGMP and cAMP inhibitory actions on platelet aggregation, reflecting respective VASP phosphorylation mechanisms.³² Regardless, VASP Ser239 phosphorylation induced by cGMP is emerging as a pivotal mechanism mediating VASP redistribution and disassembly of VASP-regulated membrane protrusions, including filopodia and lamellipodia.^{14, 33-34} In that context, Ser239 is strategically located within the EVH2 domain of VASP, immediately adjacent to the G-actin binding site, which localizes VASP at filopodial tips and coordinates the transfer of profilin-bound G-actin to the barbed end of the elongating F-actin filament.³³ Phosphorylation of VASP Ser239 interferes with EVH2-dependent processes at membrane leading edges, destabilizing VASP-actin interactions and organelle protrusive dynamics.^{14, 33} Moreover, VASP Ser239, but not VASP Ser157, phosphorylation inhibits VASP-driven F-actin polymerization,¹³ It is demonstrated herein that VASP Ser239 phosphorylation suppresses the invasive actin cytoskeleton of tumor cells and represents a prototype to be exploited in innovative diagnostic and therapeutic approaches for colorectal cancer. Specifically, ligand-dependent GCC signaling may prevent colon cancer metastasis by neutralizing oncogenic VASP functions at the invasive cancer front.

The results from the experiments described herein suggest VASP, and VASP phosphorylation at Ser-157 (pSer157-VASP) and Ser-239 (pSer239-VASP) are specific molecular biomarkers for the prognosis and therapeutic targeting of patients with colorectal cancer. Indeed, expression of all the VASP species are significantly reduced in human colorectal adenocarcinomas compared to their respective normal counterparts (FIG. 3A). Of significance, pSer157-VASP and pSer239-VASP are the most compromised signals, reflected by their maximal loss of expression following carcinogenesis (FIG. 3A). Newly generated results demonstrate VASP and VASP Ser phosphorylation are key biomarkers signifying the earliest events of colorectal tumorigenesis, and colorectal cancer metastasis. They underscore the great value of VASP, pSer157-VASP and pSer239-VASP as novel prognostic and predictive factors for patients with colorectal cancer. Novel findings presented in this section reflect immunohistochemistry (IHC) with human specimens obtained from the Department of Pathology, Anatomy and Cell Biology of Thomas Jefferson University (Philadelphia, Pa., USA), under a protocol approved by the Institutional Review Board.

The significance of VASP, pSer157-VASP and pSer239-VASP as specific biomarkers for colorectal cancer is predicated upon their impact on key processes underlying the cancer cell phenotype. As demonstrated herein, studies in mice demonstrate that pSer239-VASP regulates the metastatic behavior of human colon cancer cells, supporting its role as a prognostic indicator of colorectal cancer metastasis. Also, novel in vitro investigations on tumor cell proliferation provide substantial evidences for the utility of a genetic therapy targeting VASP Ser157 as anticancer strategy to treat colorectal cancer in patients.

The results described herein suggest VASP, pSer157-VASP and pSer239-VASP are novel predictors of tumor initiation, carcinogenesis and metastatic disease progression in colon and rectal cancer. These concepts could be translated into novel diagnostic methodologies to accurately stage patients, and select their appropriate clinical or therapeutic management. Also, aspects of the invention propose VASP, pSer157-VASP and pSer239-VASP are innovative therapeutic targets for patients with colorectal cancer. Further, these molecules could be employed as surrogate biomarkers of efficacy for novel chemotherapeutics discovery in colorectal cancer. Finally, aspects of the invention, both diagnostic and therapeutic, can be extended to other cancers of epithelial origin (e.g., breast, prostate, lung, liver, pancreas, kidney, ovary, gastric, oral, esophageal).

### Materials and Methods

Reagents.

ST was prepared as described,⁵ while 8-br-cGMP was obtained from Sigma-Aldrich (St. Louis, Mo.). Antibodies to human VASP, phosphorylated VASP at Ser157, GAPDH, villin and cortactin were from Santa Cruz Biotechnology (Santa Cruz, Calif.). Two anti-phosphorylated VASP at Ser239 antibodies were purchased, including clone 16C2 from Abcam (Cambridge, Mass.) and a rabbit antibody (SAB-4300129) from Sigma-Aldrich. The anti-human guanylin antibody was from BioDesign International (Saco, Me.), while the anti-human PKG was obtained from Assay Designs (Ann Arbor, Mich.). The antibody to the human GCC was obtained from Rockland Immunochemicals (Gilbertsville, Pa.). DQ-collagen IV, Alexa fluor 555 anti-rabbit IgG, Alexa fluor 633 anti-mouse IgG, Oregon Green 488 phalloidin and Slowfade Gold with DAPI were obtained from Invitrogen (Carlsbad, Calif.). Matrigel was from BD Bioscience (Bedford, Mass.), the general matrix metalloproteinase (MMP) inhibitor GM1006 from Calbiochem (San Diego, Calif.), and all the reagents for cell culture from Mediatech Inc. (Herndon, Va.). Scinti Verse was obtained from Fischer Scientific (Fair Lawn, N.J.). FBS and DMEM/F12 and all the reagents for cell culture were from Mediatech Inc. (Herndon, Va.). Native ST was prepared by solid phase synthesis and purified by reverse phase HPLC, their structure confirmed by mass spectrometry, and their activities confirmed by examining competitive ligand binding and guanylyl cyclase activation. [methyl-H]Thymidine (1 mCi/ml) was obtained from Amersham Pharmacia Biotech Inc. (Piscataway, N.J.). All other chemicals were obtained from Sigma-Aldrich (St. Louis, Mo.).³

Clinical Specimens and Immunohistochemistry.

Paraffin-embedded specimens from 7 patients (Table 2) with histologically-confirmed adenocarcinomas of the colo-rectum were obtained from the Department of Pathology, Anatomy and Cell Biology of Thomas Jefferson University (Philadelphia, Pa.) under a protocol approved by the Institutional Review Board and subjected to immunohistochemistry (IHC). Briefly, following deparaffinization and rehydratation (with xylene/ethanol/water washes), antigens in tissue sections (5 μm) were unmasked by two consecutive heating cycles (100° C. for 5 min in 10 mM citric buffer, pH 6.0). Then, specimens were incubated overnight (4° C.) with antibodies (1:100 dilution, unless otherwise indicated) against either human guanylin, GCC, PKG, VASP, and phosphorylated VASP at Ser157 or Ser239 followed by incubation with the respective secondary antibody and DAB substrate (Avidin-biotin kit; Vector Laboratory, Burlingame, Calif.). Staining intensity was quantified by a blinded pathologist on a 1 to 3 scale (1, low or absent; 2, medium; 3 high).

Cell Culture.

All the human colon carcinoma cell lines investigated were purchased from the American Type Culture Collection (ATCC, Manassas, Va.) and used within 6 months from resuscitation. Methods of cell authentication by ATCC include analyses of morphological and growth characteristics, isoenzymology and DNA profiles. T84 and HCT-116 human colon carcinoma cells were cultured (37° C., 5% CO2) with 10% fetal bovine serum in DMEM/F12 or DMEM, respectively. Cells (2-20 passages) were fed with fresh medium every third day and split when sub-confluent. Cancer cells (2-20 passages) were cultured (37° C., 5% CO2) with 10% FBS in DMEM/F12, fed with fresh medium every third day and split when sub-confluent.

TABLE 2
Clinical Data for Colorectal Cancer Patients

| Age (y) | |
|---|---|
| Median (range) | 64 (46-77) |
| Gender (%) | |
| Male | 5 (71.4) |
| Female | 2 (28.6) |
| Tumor Site (%) | |
| Colon | 6 (85.7) |
| Rectum | 1 (14.3) |
| Differentiation Grade (%) | |
| Well | 1 (14.3) |
| Moderate | 5 (71.4) |
| Poor | 1 (14.3) |
| Tumor Depth * (%) | |
| Tis | 0 (0) |
| T1 | 0 (0) |
| T2 | 1 (14.3) |
| T3 | 6 (85.7) |
| T4 | 0 (0) |
| Lymph Node Metastasis (%) | |
| Yes | 3 (42.9) |
| No | 4 (57.1) |

*Tis, limited to mucosa (carcinoma in situ);
T1, limited to submucosa;
T2, invading the muscularis propria;
T3, invading the serosa;
T4, invading adjacent organs.

Plasmids and Cell Transductions.

Full length VASP cDNA (Invitrogen, Carlsbad, Calif.) was sub-cloned into the XhoI-EcoR1 multiple cloning site of mouse stem cell virus (MSCV)-puro retroviral vector. Point mutations of VASP serines 157 (tcc→gcc) and 239 (agc→gcc) were performed employing the QuikChange XL Site-Directed Mutagenesis kit (Stratagene, La Jolla, Calif.) to generate VASP mutants with alanine substitutions at Ser157 (S157A), Ser239 (S239A) or both (AA). Green fluorescent protein (GFP)-VASP constructs were generated by sub-cloning wild type VASP or the VASP mutant S239A in-frame to the C-terminus of GFP using the pRetroQ-AcGFP—C1 vector (Stratagene). HEK 293T17 cells (from ATCC) were transfected with 1 μg of each purified MSCV construct plus 1 μg of the packaging vector pC1-Ampho employing Fugene transfection reagent (Roche, Base1, Switzerland). T84 cells were transduced (for 72 h) with viral supernatants supplemented with 4 μg/ml polybrene, then selected and maintained in 5 μg/ml puromycin. In this way, the following T84 clones stably-expressing the MSCV-driven gene were generated: VASP, VASP-S157A, VASP-S239A, VASP-AA, GFP, GFP-VASP and GFP-VASP-S239A.

Immunoblot Analysis.

Protein samples (40 μg in SDS loading buffer) were separated by electrophoresis on 10% acrylamide Tris-Glycine gels, transferred to nitrocellulose membranes, and probed (1:1,000 each) with rabbit polyclonal antibodies against VASP, phosphorylated VASP at Ser157, GAPDH, villin or mouse monoclonal antibody against phosphorylated VASP at Ser239 in TBS-Tween (5% milk) overnight at 4° C. After washing the primary antibody, membranes were probed with the appropriate secondary antibody (1:2,000) (Santa Cruz Biotechnology) for 1 h at room temperature Immunostained bands were imaged with a Kodak Image Station 4000R and quantified by densitometry.

Immunofluorescence and Imaging.

Filopodia dynamics were studied in cancer cells cultured on Lab-Tek glass microscope-chambered slides and treated for 2 h with PBS (control), the GCC ligand ST (1 μM) or the cGMP analog 8-br-cGMP (5 mM). Invadopodia were examined employing glass coverslips coated with 25 μl Matrigel and incubated at 37° C. (10 min) to solidify. Here, cancer cells were plated drop-wise directly onto Matrigel, allowed to firmly adhere (1 h) and equilibrate (24 h) in their culturing medium, and finally treated (2 h) with PBS or ST (1 μM). Following treatments, cells were fixed (15 min) with ice-cold paraformaldehyde (4%, in PBS) and permeabilized (10 min) with 0.1% Triton X-100. Following overnight incubations (4° C.; in PBS containing 2% bovine serum albumin) with mouse anti-cortactin and/or rabbit anti-VASP antibodies (1:100 each) and 5 units/ml Oregon Green 488 phalloidin, cells were incubated (60 min at room temperature) with Alexa fluor 633 anti-mouse IgG and/or Alexa fluor 555 anti-rabbit IgG. Specificity of antigen/antibody reactions was assessed employing the secondary antibody alone. Some preparations were counterstained with Slowfade Gold anti-fade reagent with DAPI to identify cell nuclei. Images (63×) were acquired with a confocal laser scanning microscope (LSM 510, Carl Zeiss, Jena, Germany) equipped with a monochrome CCD camera and pseudo-colored with image-analysis software.

For filopodia measurements, cells were analyzed by differential interference contrast (DIC) microscopy. Filopodial protrusions (≧5 μM) were examined on 8-10 random cell colonies per treatment (˜50-100 filopodia/treatment) by quantifying the number (per cell colony perimeter) and length (the average distance from the cell base to the tip) of filopodia. The cancer cell colony perimeter (˜264±41 μM) was determined to ensure that results from colonies with different sizes could be directly compared. Measurements were performed using the Zeiss LSM Image Browser software (Carl Zeiss Microimaging, Thornwood, N.Y.). For invadopodia analyses, the number and length of individual invadopodia (the baso-lateral actin-based membrane projections) per cancer cell were quantified (with the Zeiss LSM Image Browser software) on 8-10 random cells per treatment from 3-dimensional (3-D) reconstructions created from orthogonal X, Y and Z sections of complete Z-stack cell images (at 1 μm increments) of the confocal microscope. Finally, live cell microscopy was performed on culture dishes (fitted on a heated stage) of GFP-VASP (n=6) or GFP-VASP-S239A (n=7) cells by acquiring time-lapse images at 0, 1, 5, 10 and 15 min after treatment with the confocal microscope. Analysis of filopodia was performed with Image J (NIH, Bethesda, Md.). The GFP-based fluorescent signal was quantified at filopodia tips (the distal ¼ of each filopodia), along the entire length of each filopodium and throughout the cell body, and normalized to the background fluorescence.

DQ-Collagen IV Degradation.

Glass coverslips were coated with 20 μl Matrigel containing 25 μg/ml DQ-collagen IV and incubated at 37° C. (10 min) to solidify. Cancer cells were plated drop-wise directly onto these Matrigel scaffolds, allowed to firmly adhere (1 h), and treated (24 h) in their culturing medium with PBS or ST (1 μM). Then, cells were fixed in methanol (−20° C. for 15 min) and processed by confocal microscopy to generate Z-stack images, as described for invadopodia analyses above. DQ-collagen IV degradation was quantified in 8-10 cells per treatment with the NIH Image J as the mean fluorescent intensity of the fluorescent cleavage product (from DQ-collagen IV) over the cell and pericellular area per cell area. Results were normalized to the background fluorescence.

Mouse Peritoneal Carcinomatosis.

Cancer cells treated in vitro were injected (1×10 per ml in serum-free medium) into the lower-right peritoneal cavity of lightly anesthetized male Cr:NIH-bg-nu-Xid mice (5 wk old; NCI, Fredrick, Md.). Animals were examined twice weekly to check for signs of infection or other difficulties. At 12 weeks post surgery animals were sacrificed and the peritoneal cavity examined. Peritoneal carcinomatosis was scored using a modified clinical carcinomatosis index. The peritoneal cavity was divided into 10 regions and each region was assigned a score corresponding to the presence and size of visible xenografts. Regions with no visible tumors were scored as 0, those with lesions<0.5 mm were scored as 1, those with lesion>0.5 mm and <1.5 mm were scored as 2, and those with lesions>1.5 mm were scored as 3. Scores from individual regions were summed to obtain the overall carcinomatosis score.⁷

Cell Proliferation.

Cell number was quantified on FBS-stimulated cultures (in 6 well plates) with a hemocytometer, following trypsinization and staining with trypan blue. H-Thymidine incorporation into DNA was quantified in 96 well plates by synchronizing cells (FBS starvation for 48 h), stimulating cell cycle progression with optimum media (10% FBS in DMEM/F12), and incubating cells with 0.2 μCi/well of H-thymidine (final 3 h). Following incubation, media was aspirated, cells were incubated for 15 min with ice-cold 10% TCA and rinsed sequentially with 10% TCA and 100% methanol. The acid-insoluble material containing H-labeled DNA was solubilized in 100 μL of 0.2 N NaOH, 80 μL aliquots were dissolved in 1 ml ScintiVerse and radioactivity quantified in a Packard p-scintillation spectrometer.

Statistical Analysis.

Unless otherwise indicated, data are mean±SEM of ≧3 independent experiments. Statistical analyses were performed with the Student's t test.

### REFERENCES

1. Pitari G M, Li P, Lin J E, Zuzga D, Gibbons A V, Snook A E, Schulz S, Waldman S A. The paracrine hormone hypothesis of colorectal cancer. Clin Pharmacol Ther 2007; 82:441-7.
2. Steinbrecher K A, Tuohy T M, Heppner Goss K, Scott M C, Witte D P, Groden J, Cohen M B. Expression of guanylin is downregulated in mouse and human intestinal adenomas, Biochem Biophys Res Commun 2000; 273:225-30.
3. Notterman D A, Alon U, Sierk A J, Levine A J. Transcriptional gene expression profiles of colorectal adenoma, adenocarcinoma, and normal tissue examined by oligonucleotide arrays. Cancer Res 2001; 61:3124-30.
4. Lucas K A, Pitari G M, Kazerounian S, Ruiz-Stewart I, Park J, Schulz S, Chepenik K P, Waldman S A. Guanylyl cyclases and signaling by cyclic GMP. Pharmacol Rev 2000; 52:375-414.
5. Pitari G M, Di Guglielmo M D, Park J, Schulz S, Waldman S A. Guanylyl cyclase C agonists regulate progression through the cell cycle of human colon carcinoma cells. Proc Natl Acad Sci USA 2001; 98:7846-51.
6. Lin J E, Li P, Snook A E, Schulz S, Dasgupta A, Hyslop T M, Gibbons A V, Marszlowicz G, Pitari G M, Waldman S A. The hormone receptor GUCY2C suppresses intestinal tumor formation by inhibiting AKT signaling. Gastroenterology 2010; 138:241-54.
7. Li P, Schulz S, Bombonati A, Palazzo J P, Hyslop T M, Xu Y, Barab A A, Siracusa L D, Pitari G M, Waldman S A. Guanylyl cyclase C suppresses intestinal tumorigenesis by restricting proliferation and maintaining genomic integrity. Gastroenterology 2007; 133:599-607.
8. Deguchi A, Soh J W, Li H, Pamukcu R, Thompson W J, Weinstein I B. Vasodilator-stimulated phosphoprotein (VASP) phosphorylation provides a biomarker for the action of exisulind and related agents that activate protein kinase G. Mol Cancer Ther 2002; 1:803-9.
9. Krause M, Dent E W, Bear J E, Loureiro J J, Gertler F B, Ena/VASP proteins: regulators of the actin cytoskeleton and cell migration. Annu Rev Cell Dev Biol 2003; 19:541-64.
10. Mejillano M R, Kojima S, Applewhite D A, Gertler F B, Svitkina T M, Borisy G G. Lamellipodial versus filopodial mode of the actin nanomachinery: pivotal role of the filament barbed end. Cell 2004; 118:363-73.
11. Butt E, Abel K, Krieger M, Palm D, Hoppe V, Hoppe J, Walter U. cAMP- and cGMP-dependent protein kinase phosphorylation sites of the focal adhesion vasodilator-stimulated phosphoprotein (VASP) in vitro and in intact human platelets, J Biol Chem 1994; 269:14509-17.
12. Blume C, Benz μM, Walter U, Ha J, Kemp B E, Renne T. AMP-activated protein kinase impairs endothelial actin cytoskeleton assembly by phosphorylating vasodilator-stimulated phosphoprotein. J Biol Chem 2007; 282:4601-12.
13. Benz μM, Blume C, Seifert S, Wilhelm S, Waschke J, Schuh K, Gertler F, Munzel T, Renne T. Differential VASP phosphorylation controls remodeling of the actin cytoskeleton. J Cell Sci 2009; 122:3954-65.
14. Lindsay S L, Ramsey S, Aitchison M, Renne T, Evans T J. Modulation of lamellipodial structure and dynamics by NO-dependent phosphorylation of VASP Ser239. J Cell Sci 2007; 120:3011-21.
15. Yilmaz M, Christofori G. EMT, the cytoskeleton, and cancer cell invasion. Cancer Metastasis Rev 2009; 28:15-33.
16. Yamaguchi H, Condeelis J. Regulation of the actin cytoskeleton in cancer cell migration and invasion. Biochim Biophys Acta 2007; 1773:642-52.
17. Dostmann W R, Taylor M S, Nickl C K, Brayden J E, Frank R, Tegge W J. Highly specific, membrane-permeant peptide blockers of cGMP-dependent protein kinase Ialpha inhibit NO-induced cerebral dilation. Proc Natl Acad Sci USA 2000; 97:14772-7.
18. Bear J E, Gertler F B. Ena/VASP: towards resolving a pointed controversy at the barbed end. J Cell Sci 2009; 122:1947-53.
19. Steeg P S. Tumor metastasis: mechanistic insights and clinical challenges. Nat Med 2006; 12:895-904.
20. Linder S. The matrix corroded: podosomes and invadopodia in extracellular matrix degradation. Trends Cell Biol 2007; 117:107-17.
21. Lubbe W J, Zuzga D S, Zhou Z, Fu W, Pelta-Heller J, Muschel R J, Waldman S A, Pitari G M. Guanylyl cyclase C prevents colon cancer metastasis by regulating tumor epithelial cell matrix metalloproteinase-9. Cancer Res 2009; 69:3529-36.
22. Applewhite D A, Barzik M, Kojima S, Svitkina T M, Gertler F B, Borisy G G. Ena/VASP proteins have an anti-capping independent function in filopodia formation. Mol Biol Cell 2007; 18:2579-91.
23. Philippar U, Roussos E T, Oser M, Yamaguchi H, Kim H D, Giampieri S, Wang Y, Goswami S, Wyckoff J B, Lauffenburger D A, Sahai E, Condeelis J S, et al. A Mena invasion isoform potentiates EGF-induced carcinoma cell invasion and metastasis. Dev Cell 2008; 15:813-28.
24. Han G, Fan B, Zhang Y, Zhou X, Wang Y, Dong H, Wei Y, Sun S, Hu M, Zhang J, Wei L. Positive regulation of migration and invasion by vasodilator-stimulated phosphoprotein via Racl pathway in human breast cancer cells. Oncol Rep 2008; 20:929-39.
25. Dertsiz L, Ozbilim G, Kayisli Y, Gokhan G A, Demircan A, Kayisli U A. Differential expression of VASP in normal lung tissue and lung adenocarcinomas. Thorax 2005; 60:576-81.
26. Hu L D, Zou H F, Zhan S X, Cao K M. EVL (Ena/VASP-like) expression is up-regulated in human breast cancer and its relative expression level is correlated with clinical stages. Oncol Rep 2008; 19:1015-20.
27. Weaver A M. Invadopodia: specialized cell structures for cancer invasion. Clin Exp Metastasis 2006:23:97-105.
28. Stylli S S, Kaye A H, Lock P. Invadopodia: at the cutting edge of tumour invasion. J Clin Neurosci 2008; 15:725-37.
29. Wentworth J K, Pula G, Poole A W. Vasodilator-stimulated phosphoprotein (VASP) is phosphorylated on Ser157 by protein kinase C-dependent and -independent mechanisms in thrombin-stimulated human platelets. Biochem J 2006; 393:555-64.
30. Lebrand C, Dent E W, Strasser G A, Lanier L M, Krause M, Svitkina T M, Borisy G G, Gertler F B. Critical role of Ena/VASP proteins for filopodia formation in neurons and in function downstream of netrin-1. Neuron 2004; 42:37-49.
31. Gomez T M, Robles E. The great escape; phosphorylation of Ena/VASP by PKA promotes filopodial formation. Neuron 2004; 42:1-3.
32. Aszodi A, Pfeifer A, Ahmad M, Glauner M, Zhou X H, Ny L, Andersson K E, Kehrel B, Offermanns S, Fassler R. The vasodilator-stimulated phosphoprotein (VASP) is involved in cGMP- and cAMP-mediated inhibition of agonist-induced platelet aggregation, but is dispensable for smooth muscle function. EMBO J 1999; 18:37-48.
33. Barzik M, Kotova T I, Higgs H N, Hazelwood L, Hanein D, Gertler F B, Schafer D A. Ena/VASP proteins enhance actin polymerization in the presence of barbed end capping proteins. J Biol Chem 2005; 280:28653-62.
34. Yaroslayskiy B B, Zhang Y, Kalla S E, Garcia Palacios V, Sharrow A C, Li Y, Zaidi M, Wu C, Blair H C. NO-dependent osteoclast motility: reliance on cGMP-dependent protein kinase I and VASP. J Cell Sci 2005; 118:5479-87.

1-68. (canceled)

69. A method for determining a prognosis of a subject who has been identified as having an epithelial tumor comprising the steps of:
a) i) measuring the level of pSer239-VASP in a sample of the tumor to establish a tumor pSer239-VASP level, and/or
ii) measuring the level of pSer239-VASP in a sample of normal tissue adjacent to the tumor to establish a normal pSer239-VASP level, and/or
iii) measuring the level of pSer157-VASP in a sample of the tumor to establish a tumor pSer157-VASP level, and/or
iv) measuring the level of pSer157-VASP in a sample of normal tissue adjacent to the tumor to establish a normal pSer157-VASP level, and/or
v) measuring the level of total VASP in a sample of the tumor to establish a tumor total-VASP level, and/or
vi) measuring the level of total VASP in a sample of normal tissue adjacent to the tumor to establish a normal total-VASP level; and
b) i) calculating relative amounts of the tumor pSer239-VASP and tumor total-VASP as ratio tumor pSer239-VASP:tumor total-VASP, and/or
ii) calculating relative amounts of the normal pSer239-VASP and normal total-VASP as ratio normal pSer239-VASP:normal total-VASP, and/or
iii) calculating relative amounts of the tumor pSer157-VASP and tumor total-VASP as ratio tumor pSer157-VASP:tumor total-VASP, and/or
iv) calculating relative amounts of the normal pSer157-VASP and normal total-VASP as ratio normal pSer157-VASP:normal total-VASP, and/or
v) calculating relative amounts of the tumor pSer239-VASP and tumor pSer157-VASP as ratio tumor pSer239-VASP:tumor pSer157-VASP, and/or
vi) calculating relative amounts of the normal pSer239-VASP and normal pSer157-VASP as ratio normal pSer239-VASP:normal pSer157-VASP, and/or
vii) comparing levels of the tumor pSer239-VASP and normal pSer239-VASP, and/or
viii) comparing levels of the tumor pSer157-VASP and normal pSer157-VASP, and/or
ix) comparing levels of the tumor total-VASP and normal total-VASP, and/or
x) comparing levels of the tumor pSer239-VASP to a reference level of tumor pSer239-VASP, and/or
xi) comparing levels of the tumor pSer157-VASP and a reference level of tumor pSer157-VASP, and/or
xii) comparing levels of the tumor total-VASP and a reference level of tumor total-VASP,
xiii) comparing levels of the normal pSer239-VASP to a reference level of normal pSer239-VASP, and/or
xiv) comparing levels of the normal pSer157-VASP and a reference level of normal pSer157-VASP, and/or
xv) comparing levels of the normal total-VASP and a reference level of normal total-VASP,
wherein:
a reduction in ratio tumor pSer239-VASP:tumor total-VASP compared to ratio normal pSer239-VASP:normal total-VASP is indicative of a greater the likelihood of a negative prognosis and the greater the level of reduction indicates a greater likelihood of a negative prognosis;
a reduction in ratio tumor pSer157-VASP:tumor total-VASP compared to ratio normal pSer157-VASP:normal total-VASP is indicative of a greater the likelihood of a negative prognosis, and the greater the level of reduction indicates a greater likelihood of a negative prognosis;
a reduction in ratio tumor pSer239-VASP:tumor pSer157-VASP compared to ratio normal pSer239-VASP:normal pSer157-VASP is indicative of a greater the likelihood of a negative prognosis, and the greater the level of reduction indicates a greater likelihood of a negative prognosis;
a reduction in the level of the tumor pSer239-VASP compared to the level of normal pSer239-VASP is indicative of a greater the likelihood of a negative prognosis, and the greater the level of reduction indicates a greater likelihood of a negative prognosis;
a reduction in the level of the tumor pSer157-VASP compared to the level of normal pSer157-VASP is indicative of a greater the likelihood of a negative prognosis, and the greater the level of reduction indicates a greater likelihood of a negative prognosis; and
a reduction in the level of the tumor total-VASP compared to the level of normal total-VASP is indicative of a greater the likelihood of a negative prognosis, and the greater the level of reduction indicates a greater likelihood of a negative prognosis; and
a reduction in the level of the tumor pSer239-VASP compared to the reference level of tumor pSer239-VASP is indicative of a greater the likelihood of a negative prognosis, and the greater the level of reduction indicates a greater likelihood of a negative prognosis;
a reduction in the level of the tumor pSer157-VASP compared to the reference level of tumor pSer157-VASP is indicative of a greater the likelihood of a negative prognosis, and the greater the level of reduction indicates a greater likelihood of a negative prognosis; and
a reduction in the level of the tumor total-VASP compared to the reference level of tumor total-VASP is indicative of a greater the likelihood of a negative prognosis, and the greater the level of reduction indicates a greater likelihood of a negative prognosis; and
a reduction in the level of the normal pSer239-VASP compared to the reference level of normal pSer239-VASP is indicative of a greater the likelihood of a negative prognosis, and the greater the level of reduction indicates a greater likelihood of a negative prognosis;
a reduction in the level of the normal pSer157-VASP compared to the reference level of normal pSer157-VASP is indicative of a greater the likelihood of a negative prognosis, and the greater the level of reduction indicates a greater likelihood of a negative prognosis; and
a reduction in the level of the normal total-VASP compared to the reference level of normal total-VASP is indicative of a greater the likelihood of a negative prognosis, and the greater the level of reduction indicates a greater likelihood of a negative prognosis.

70. The method of claim 69 comprising the steps of:
a) i) measuring the level of pSer239-VASP in a sample of the tumor to establish a tumor pSer239-VASP level, and
ii) measuring the level of pSer239-VASP in a sample of normal tissue adjacent to the tumor to establish a normal pSer239-VASP level, and
iii) measuring the level of pSer157-VASP in a sample of the tumor to establish a tumor pSer157-VASP level, and
iv) measuring the level of pSer157-VASP in a sample of normal tissue adjacent to the tumor to establish a normal pSer157-VASP level, and
v) measuring the level of total VASP in a sample of the tumor to establish a tumor total-VASP level, and
vi) measuring the level of total VASP in a sample of normal tissue adjacent to the tumor to establish a normal total-VASP level.

71. The method of claim 69 comprising the steps of:
a) measuring the level of pSer239-VASP in a sample of the tumor to establish a tumor pSer239-VASP level, and
measuring the level of pSer239-VASP in a sample of normal tissue adjacent to the tumor to establish a normal pSer239-VASP level; and
b) comparing levels of the tumor pSer239-VASP and normal pSer239-VASP.

72. The method of claim 69 wherein:
i) the level of pSer239-VASP in a sample of the tumor is measured by
transforming pSer239-VASP in a sample of the tumor into a tumor pSer239-VASP detectable targets, and
measuring the level of tumor pSer239-VASP detectable targets; and/or
ii) the level of pSer239-VASP in a sample of normal tissue adjacent to the tumor is measured by
transforming pSer239-VASP in a sample of the normal tissue adjacent to the tumor into a normal pSer239-VASP detectable targets and
measuring the level of tumor pSer239-VASP detectable targets; and/or
iii) the level of pSer157-VASP in a sample of the tumor is measured by
transforming pSer157-VASP in a sample of the tumor into a tumor pSer157-VASP detectable targets and
measuring the level of tumor pSer157-VASP detectable targets; and/or
iv) the level of pSer157-VASP in a sample of normal tissue adjacent to the tumor is measured by
transforming pSer157-VASP in a sample of normal tissue adjacent to the tumor into a normal pSer157-VASP detectable targets and
measuring the level of normal pSer157-VASP detectable targets; and/or
v) the level of total-VASP in a sample of the tumor is measured by
transforming total-VASP in a sample of the tumor into a tumor total-VASP detectable targets and
measuring the level of tumor total-VASP detectable targets; and/or
vi) the level of total-VASP in a sample of normal tissue adjacent to the tumor is measured by
transforming total-VASP in a sample of normal tissue adjacent to the tumor into a normal total-VASP detectable targets and
measuring the level of normal total-VASP detectable targets.

73. The method of claim 72 wherein:
i) the level of pSer239-VASP in a sample of the tumor is measured by
transforming pSer239-VASP in a sample of the tumor into a tumor pSer239-VASP detectable targets, and
measuring the level of tumor pSer239-VASP detectable targets; and
ii) the level of pSer239-VASP in a sample of normal tissue adjacent to the tumor is measured by
transforming pSer239-VASP in a sample of the normal tissue adjacent to the tumor into a normal pSer239-VASP detectable targets and
measuring the level of tumor pSer239-VASP detectable targets; and
iii) the level of pSer157-VASP in a sample of the tumor is measured by
transforming pSer157-VASP in a sample of the tumor into a tumor pSer157-VASP detectable targets and
measuring the level of tumor pSer157-VASP detectable targets; and
iv) the level of pSer157-VASP in a sample of normal tissue adjacent to the tumor is measured by
transforming pSer157-VASP in a sample of normal tissue adjacent to the tumor into a normal pSer157-VASP detectable targets and
measuring the level of normal pSer157-VASP detectable targets; and
v) the level of total-VASP in a sample of the tumor is measured by
transforming total-VASP in a sample of the tumor into a tumor total-VASP detectable targets and
measuring the level of tumor total-VASP detectable targets; and
vi) the level of total-VASP in a sample of normal tissue adjacent to the tumor is measured by
transforming total-VASP in a sample of normal tissue adjacent to the tumor into a normal total-VASP detectable targets and
measuring the level of normal total-VASP detectable targets.

74. The method of claim 69 wherein:
the level of pSer239-VASP in a sample of the tumor is measured by
transforming pSer239-VASP in a sample of the tumor into a tumor pSer239-VASP detectable targets, and
measuring the level of tumor pSer239-VASP detectable targets; and
the level of pSer239-VASP in a sample of normal tissue adjacent to the tumor is measured by
transforming pSer239-VASP in a sample of the normal tissue adjacent to the tumor into a normal pSer239-VASP detectable targets and
measuring the level of tumor pSer239-VASP detectable targets.

75. The method of claim 69 further comprising the step of:
prior to step a), removing samples of the tumor and samples of normal tissue adjacent to the tumor from the subject.

76. The method of claim 69 wherein the increased likelihood of a negative prognosis is an increased likelihood that the tumor will metastasize.

77. The method of claim 69, wherein the level of tumor pSer157-VASP and/or normal pSer157-VASP and/or tumor pSer239-VASP and/or normal pSer239-VASP and/or tumor total-VASP and/or normal total-VASP is determined by immunodetection.

78. The method of claim 69, wherein the level of tumor pSer157-VASP and/or normal pSer157-VASP and/or tumor pSer239-VASP and/or normal pSer239-VASP and/or tumor total-VASP and/or normal total-VASP is measured by quantitation of percentage of epithelial cell compartments with complete loss of pSer239-VASP, pSer157-VASP, and/or total VASP in the epithelial tumor, or by quantitation of intensity level detected for pSer239-VASP, pSer157-VASP, and/or total VASP multiplied by the percentage of epithelial cell compartments with that intensity level for the pSer239-VASP, pSer157-VASP, and/or total VASP, respectively.

79. The method of claim 69, wherein the epithelial tumor is in a tissue selected from the group consisting of colon, rectum, lung, breast, prostate, liver, pancreas, kidney, ovary, stomach, intestine, oral cavity, esophagus, lip, bowel, bladder, cervix, and skin of the subject.

80. The method of claim 69, wherein the epithelial tumor is a basal cell carcinoma, adenocarcinoma, renal cell carcinoma or a colorectal adenocarcinoma.

81. The method of claim 69, further comprising measuring the level of one or more additional tumor biomarkers in the epithelial tumor.

82. The method of claim 81, wherein the one or more additional tumor biomarkers includes MMP-9.

83. The method of claim 69, wherein
the level of tumor pSer157-VASP and/or the level of normal pSer157-VASP is measured by immunofluorescence or immunohistochemistry using an antibody specific for the pSer157-VASP, and/or
the level of tumor pSer239-VASP and/or the level of normal pSer239-VASP is measured by immunofluorescence or immunohistochemistry using an antibody specific for the pSer239-VASP, and/or
the level of tumor total-VASP and/or the level of normal total-VASP is measured by immunofluorescence or immunohistochemistry using an antibody specific for the total VASP

84. The method of claim 69, wherein
the level of tumor pSer157-VASP and/or the level of normal pSer157-VASP is measured by immunofluorescence or immunohistochemistry using an antibody specific for the pSer157-VASP selected from the group consisting of antibody 5C6, antibody F-3 and antibody A-7, and/or
the level of tumor pSer239-VASP and/or the level of normal pSer239-VASP is measured by immunofluorescence or immunohistochemistry using an antibody specific for pSer239-VASP wherein said antibody specific for the pSer239-VASP is antibody 16C2, and/or
the level of tumor total-VASP and/or the level of normal total-VASP is measured by immunofluorescence or immunohistochemistry using an antibody specific for total VASP, wherein said antibody specific for total VASP is selected from the group consisting of, antibody C-17, antibody S-19, antibody A-11, and antibody H-90.

85. The method of claim 69, wherein the level of tumor pSer157-VASP and/or the level of normal pSer157-VASP and/or the level of tumor pSer239-VASP and/or the level of normal pSer239-VASP and/or the level of tumor total-VASP and/or the level of normal total-VASP is expressed as a numerical value in which 0 is the level corresponding to an undetectable level and a lower level is a level closer to 0 than to the maximum level.

86. The method of claim 69, wherein the level of tumor pSer157-VASP and/or the level of normal pSer157-VASP and/or the level of tumor pSer239-VASP and/or the level of normal pSer239-VASP and/or the level of tumor total-VASP and/or the level of normal total-VASP is expressed as a numerical value ranging from 0 to 3, wherein 0 is the level corresponding to an undetectable level, 3 is the maximum level.

87. A method of treating a subject who has been identified as having an epithelial tumor comprising the steps of:
determining a prognosis of the subject by the method of claim 69,
determining whether or not the subject has a likelihood of a negative prognosis, and
administering therapeutics to said subject is said subject is determined to have a likelihood of a negative prognosis.

88. A kit for determining a prognosis of a subject who has been identified as having an epithelial tumor comprising an antibody that binds to pSer239-VASP, an antibody that binds to pSer157-VASP, an antibody that binds to total-VASP, and instructions for performing the method of claim 69 using said antibodies.
